SEQUENCE LISTING (464 sequences)

SEQ ID NO: 1 - DNA, 30 bp, synthetic construct
  source 1..30: mol_type = other DNA
gcgttttgtcgctgaggcttgctaaccgat

SEQ ID NO: 2 - DNA, 32 bp, synthetic construct
  source 1..32: mol_type = other DNA
taaacagcaaccatcgcccacgcaagggagtt

SEQ ID NO: 3 - DNA, 32 bp, synthetic construct
  source 1..32: mol_type = other DNA
attaagagcttttgcgggatcgtccgccgaca

SEQ ID NO: 4 - DNA, 30 bp, synthetic construct
  source 1..30: mol_type = other DNA
acaacttttttctgtatgggataccctcag

SEQ ID NO: 5 - DNA, 30 bp, synthetic construct
  source 1..30: mol_type = other DNA
tatcatacagaaaacgagaatgcaaatatc

SEQ ID NO: 6 - DNA, 42 bp, synthetic construct
  source 1..42: mol_type = other DNA
tccccctcacgaaccagaccggaagttttcaaactccaacag

SEQ ID NO: 7 - DNA, 32 bp, synthetic construct
  source 1..32: mol_type = other DNA
gtcaatcacaggtctttaccctgatcaaaaag

SEQ ID NO: 8 - DNA, 30 bp, synthetic construct
  source 1..30: mol_type = other DNA
gcttgagatggtttttcaactttaatcatt

SEQ ID NO: 9 - DNA, 14 bp, synthetic construct
  source 1..14: mol_type = other DNA
tgctggtaaacagg

SEQ ID NO: 10 - DNA, 26 bp, synthetic construct
  source 1..26: mol_type = other DNA
acagtattaaactcatggattttaat

SEQ ID NO: 11 - DNA, 35 bp, synthetic construct
  source 1..35: mol_type = other DNA
ttgagaatctaccgccaattttcaaagttcctgat

SEQ ID NO: 12 - DNA, 27 bp, synthetic construct
  source 1..27: mol_type = other DNA
agaaggagtgcccgaacgttattagcc

SEQ ID NO: 13 - DNA, 20 bp, synthetic construct
  source 1..20: mol_type = other DNA
aaaaacgtcctttatcaaac

SEQ ID NO: 14 - DNA, 32 bp, synthetic construct
  source 1..32: mol_type = other DNA
gtcaggatgaggtcatttttgcggttagaacc

SEQ ID NO: 15 - DNA, 32 bp, synthetic construct
  source 1..32: mol_type = other DNA
taaaactacagaaaagccccaaaattgtaaac

SEQ ID NO: 16 - DNA, 32 bp, synthetic construct
  source 1..32: mol_type = other DNA
ttcctgtatttgttaaaattcgcataccccgg

SEQ ID NO: 17 - DNA, 30 bp, synthetic construct
  source 1..30: mol_type = other DNA
gccggagaaagattcaaaagggttaaattt

SEQ ID NO: 18 - DNA, 32 bp, synthetic construct
  source 1..32: mol_type = other DNA
gaacgccaatcagctcattttttatgagtaat

SEQ ID NO: 19 - DNA, 32 bp, synthetic construct
  source 1..32: mol_type = other DNA
tattcaacttttaaatgcaatgccttgctcct

SEQ ID NO: 20 - DNA, 32 bp, synthetic construct
  source 1..32: mol_type = other DNA
aaagaagtagactggatagcgtccaccaatag

SEQ ID NO: 21 - DNA, 13 bp, synthetic construct
  source 1..13: mol_type = other DNA
taataactttgac

SEQ ID NO: 22 - DNA, 31 bp, synthetic construct
  source 1..31: mol_type = other DNA
ggcagataatagataatacatttaattgagg

SEQ ID NO: 23 - DNA, 47 bp, synthetic construct
  source 1..47: mol_type = other DNA
taataggagccgtctcaccagtttttcacacgaccaggtgaggccac

SEQ ID NO: 24 - DNA, 45 bp, synthetic construct
  source 1..45: mol_type = other DNA
ccgtcgagagggttgatatcggaacctattattttttctgaaaca

SEQ ID NO: 25 - DNA, 53 bp, synthetic construct
  source 1..53: mol_type = other DNA
tgaaagtattaagtagcggggttttgctccaccctcagaaccgcccctcattt

SEQ ID NO: 26 - DNA, 32 bp, synthetic construct
  source 1..32: mol_type = other DNA
aaccgccaagagaaggattaggataggctgag

SEQ ID NO: 27 - DNA, 30 bp, synthetic construct
  source 1..30: mol_type = other DNA
tcatacacgccaccagaaccaccaccaccc

SEQ ID NO: 28 - DNA, 30 bp, synthetic construct
  source 1..30: mol_type = other DNA
aagtacagaaggcaccaacctaagtaagcg

SEQ ID NO: 29 - DNA, 42 bp, synthetic construct
  source 1..42: mol_type = other DNA
cgggtaaaaaggagtgtactggtaatttttaagttttaacgg

SEQ ID NO: 30 - DNA, 26 bp, synthetic construct
  source 1..26: mol_type = other DNA
agttttacatcgccaacgctttttca

SEQ ID NO: 31 - DNA, 36 bp, synthetic construct
  source 1..36: mol_type = other DNA
gagtaacaatacagtaacacagtagggccaacatgt

SEQ ID NO: 32 - DNA, 32 bp, synthetic construct
  source 1..32: mol_type = other DNA
ggtcagtgtgccccctgcctatttgcgtaacg

SEQ ID NO: 33 - DNA, 32 bp, synthetic construct
  source 1..32: mol_type = other DNA
aacggctaagacagcatcggaacggacgttag

SEQ ID NO: 34 - DNA, 32 bp, synthetic construct
  source 1..32: mol_type = other DNA
tagaaaggttttgtcgtctttccaccgtataa

SEQ ID NO: 35 - DNA, 32 bp, synthetic construct
  source 1..32: mol_type = other DNA
catgttaccagaggctttgaggacagggtagc

SEQ ID NO: 36 - DNA, 12 bp, synthetic construct
  source 1..12: mol_type = other DNA
ataatcataata

SEQ ID NO: 37 - DNA, 26 bp, synthetic construct
  source 1..26: mol_type = other DNA
aaagggacacctgaaagcgtaacaac

SEQ ID NO: 38 - DNA, 20 bp, synthetic construct
  source 1..20: mol_type = other DNA
cccttctgattctggccaac

SEQ ID NO: 39 - DNA, 34 bp, synthetic construct
  source 1..34: mol_type = other DNA
taataagaataccagtataaagggagattttcag

SEQ ID NO: 40 - DNA, 32 bp, synthetic construct
  source 1..32: mol_type = other DNA
ataatggaagggtaaaacattgcaaatccaat

SEQ ID NO: 41 - DNA, 26 bp, synthetic construct
  source 1..26: mol_type = other DNA
aaagtaaaaaacaaaattgctttgaa

SEQ ID NO: 42 - DNA, 30 bp, synthetic construct
  source 1..30: mol_type = other DNA
taagtcctgaattaaaataatatcccatcc

SEQ ID NO: 43 - DNA, 35 bp, synthetic construct
  source 1..35: mol_type = other DNA
aagaagattattcatttcaattatttgaaaccgaa

SEQ ID NO: 44 - DNA, 14 bp, synthetic construct
  source 1..14: mol_type = other DNA
aatgcagagagcaa

SEQ ID NO: 45 - DNA, 30 bp, synthetic construct
  source 1..30: mol_type = other DNA
ctaaaggtctatcagggcgatgattaaatg

SEQ ID NO: 46 - DNA, 42 bp, synthetic construct
  source 1..42: mol_type = other DNA
tccaacgtcttctccgtgggaacaattttacggcggattgac

SEQ ID NO: 47 - DNA, 32 bp, synthetic construct
  source 1..32: mol_type = other DNA
cgtaatggtaaccgtgcatctgcccctgtgtg

SEQ ID NO: 48 - DNA, 32 bp, synthetic construct
  source 1..32: mol_type = other DNA
accaccggccgccaccctcagagccaccagag

SEQ ID NO: 49 - DNA, 32 bp, synthetic construct
  source 1..32: mol_type = other DNA
cgcagtctagcattgacaggaggtgccgccac

SEQ ID NO: 50 - DNA, 30 bp, synthetic construct
  source 1..30: mol_type = other DNA
aatgaaattagcaaggccggaatgaggcag

SEQ ID NO: 51 - DNA, 32 bp, synthetic construct
  source 1..32: mol_type = other DNA
aataaatcgattggccttgatattagtagcac

SEQ ID NO: 52 - DNA, 32 bp, synthetic construct
  source 1..32: mol_type = other DNA
gtagcgacagccagcaaaatcaccgaaatagc

SEQ ID NO: 53 - DNA, 32 bp, synthetic construct
  source 1..32: mol_type = other DNA
ccaataattcttaccgaagcccttgccatttg

SEQ ID NO: 54 - DNA, 42 bp, synthetic construct
  source 1..42: mol_type = other DNA
attatttatagagataacccacaagttttaattgagttaagc

SEQ ID NO: 55 - DNA, 45 bp, synthetic construct
  source 1..45: mol_type = other DNA
ttattacgcagtatgttagtttaagaaaagtttttaagcagatag

SEQ ID NO: 56 - DNA, 53 bp, synthetic construct
  source 1..53: mol_type = other DNA
ccgaacaaagttaggaatacccaaaagaaagacaccacggaataaatggttta

SEQ ID NO: 57 - DNA, 32 bp, synthetic construct
  source 1..32: mol_type = other DNA
tgtcacaagaaacgcaataataacccagaagg

SEQ ID NO: 58 - DNA, 30 bp, synthetic construct
  source 1..30: mol_type = other DNA
gggtaatctggtttgccccagcagcaagcg

SEQ ID NO: 59 - DNA, 32 bp, synthetic construct
  source 1..32: mol_type = other DNA
ccagtgaggccctgagagagttgcaggcgaaa

SEQ ID NO: 60 - DNA, 32 bp, synthetic construct
  source 1..32: mol_type = other DNA
agaattaatgatggtggttccgaatgcccttc

SEQ ID NO: 61 - DNA, 30 bp, synthetic construct
  source 1..30: mol_type = other DNA
gggtgccggaagcataaagtgtatcggcaa

SEQ ID NO: 62 - DNA, 32 bp, synthetic construct
  source 1..32: mol_type = other DNA
atagggtttataaatcaaaagaatacacaaca

SEQ ID NO: 63 - DNA, 32 bp, synthetic construct
  source 1..32: mol_type = other DNA
attgcgttatccgctcacaattccgtagatgg

SEQ ID NO: 64 - DNA, 30 bp, synthetic construct
  source 1..30: mol_type = other DNA
tgagcgaaagcaaatatttaaaacaggaag

SEQ ID NO: 65 - DNA, 32 bp, synthetic construct
  source 1..32: mol_type = other DNA
aagttgggagctttccggcaccgcagatcgca

SEQ ID NO: 66 - DNA, 53 bp, synthetic construct
  source 1..53: mol_type = other DNA
atcggcctcaggattctggtgccggaaacagggggatgtgctgcaccagtcac

SEQ ID NO: 67 - DNA, 45 bp, synthetic construct
  source 1..45: mol_type = other DNA
cattcaggctgcgcaactgagtttgaggggattttcgacgacagt

SEQ ID NO: 68 - DNA, 32 bp, synthetic construct
  misc_feature 1..32: note = L-core-68
  source 1..32: mol_type = other DNA
aagggaaggagtgttgttccagttagcccgag

SEQ ID NO: 69 - DNA, 32 bp, synthetic construct
  misc_feature 1..32: note = L-core-69
  source 1..32: mol_type = other DNA
ggcgctggatagcagcctttacagtagacggg

SEQ ID NO: 70 - DNA, 30 bp, synthetic construct
  misc_feature 1..30: note = L-core-70
  source 1..30: mol_type = other DNA
gaacctcaacgattttttgtttaagtcaga

SEQ ID NO: 71 - DNA, 32 bp, synthetic construct
  source 1..32: mol_type = other DNA
cccagctacgagcgtctttccagacacaaaca

SEQ ID NO: 72 - DNA, 32 bp, synthetic construct
  source 1..32: mol_type = other DNA
tgacccccagaatacactaaaacaatggaaag

SEQ ID NO: 73 - DNA, 32 bp, synthetic construct
  source 1..32: mol_type = other DNA
atgacaacttgataccgatagttgccaaaaaa

SEQ ID NO: 74 - DNA, 32 bp, synthetic construct
  source 1..32: mol_type = other DNA
atctaaagacagccctcatagttactgtagca

SEQ ID NO: 75 - DNA, 32 bp, synthetic construct
  source 1..32: mol_type = other DNA
taaatgaatcaacagtttcagcgggcgaataa

SEQ ID NO: 76 - DNA, 32 bp, synthetic construct
  source 1..32: mol_type = other DNA
ctcatatagcaaggataaaaatttaagccttt

SEQ ID NO: 77 - DNA, 32 bp, synthetic construct
  source 1..32: mol_type = other DNA
gtgtaggtacagtcaaatcaccatttaatgcc

SEQ ID NO: 78 - DNA, 32 bp, synthetic construct
  source 1..32: mol_type = other DNA
ttgataatgcatgtcaatcatatggatctaca

SEQ ID NO: 79 - DNA, 54 bp, synthetic construct
  source 1..54: mol_type = other DNA
gacgttgtaaaacgacggccagtgtacgccagctggcgaacaggcaaagcgcca

SEQ ID NO: 80 - DNA, 30 bp, synthetic construct
  source 1..30: mol_type = other DNA
attgtatagagaatcgatgaacagtctgga

SEQ ID NO: 81 - DNA, 32 bp, synthetic construct
  source 1..32: mol_type = other DNA
ggaattagaccgtcaccgacttgattaaaggt

SEQ ID NO: 82 - DNA, 32 bp, synthetic construct
  source 1..32: mol_type = other DNA
cattaccaccatcgatagcagcacttagcgtc

SEQ ID NO: 83 - DNA, 54 bp, synthetic construct
  source 1..54: mol_type = other DNA
tcagggatagcaagcccaataggaggaggtttagtaccgcagtaccaggcggat

SEQ ID NO: 84 - DNA, 30 bp, synthetic construct
  source 1..30: mol_type = other DNA
tcagagctcaaaatcaccggaagccatctt

SEQ ID NO: 85 - DNA, 32 bp, synthetic construct
  source 1..32: mol_type = other DNA
cctcagaaaaccgcctccctcagacattttcg

SEQ ID NO: 86 - DNA, 54 bp, synthetic construct
  source 1..54: mol_type = other DNA
ccagcgccaaagacaaaagggcgatataaaagaaacgcaactggcatgattaag

SEQ ID NO: 87 - DNA, 32 bp, synthetic construct
  source 1..32: mol_type = other DNA
aaattgttatggtcatagctgtttgctcgaat

SEQ ID NO: 88 - DNA, 32 bp, synthetic construct
  source 1..32: mol_type = other DNA
tacgagcctaatgagtgagctaacttccagtc

SEQ ID NO: 89 - DNA, 32 bp, synthetic construct
  source 1..32: mol_type = other DNA
accgcctgacgggcaacagctgatattaatga

SEQ ID NO: 90 - DNA, 30 bp, synthetic construct
  source 1..30: mol_type = other DNA
gtccacgcgccagggtggttttggtttgcg

SEQ ID NO: 91 - DNA, 20 bp, synthetic construct
  source 1..20: mol_type = other DNA
agcatcagctgaacctcaaa

SEQ ID NO: 92 - DNA, 24 bp, synthetic construct
  source 1..24: mol_type = other DNA
cggaattatcatcataaaaccacc

SEQ ID NO: 93 - DNA, 37 bp, synthetic construct
  source 1..37: mol_type = other DNA
tatcagatgatggttatcattttgcggaaaaaagttt

SEQ ID NO: 94 - DNA, 41 bp, synthetic construct
  source 1..41: mol_type = other DNA
gtttattttacgtcagatgaatcaattcatcaatacctgat

SEQ ID NO: 95 - DNA, 46 bp, synthetic construct
  source 1..46: mol_type = other DNA
gcggtcttaacaccctaaaatatttttctttaggagcacaggtgag

SEQ ID NO: 96 - DNA, 41 bp, synthetic construct
  source 1..41: mol_type = other DNA
aaggttatgcctgcaacagtgccacgctgaaagggaggatt

SEQ ID NO: 97 - DNA, 28 bp, synthetic construct
  source 1..28: mol_type = other DNA
cctcattttatcaatatctggtatctaa

SEQ ID NO: 98 - protein, 37 aa, synthetic construct
CGAACCACCAGCAGAGGCACAGACAATATTGATAGAA

SEQ ID NO: 99 - DNA, 22 bp, synthetic construct
  source 1..22: mol_type = other DNA
taagaatacgtagataaaacag

SEQ ID NO: 100 - DNA, 45 bp, synthetic construct
  source 1..45: mol_type = other DNA
taccatatctgatagccaacatcgccattaaaaattggctattag

SEQ ID NO: 101 - DNA, 33 bp, synthetic construct
  source 1..33: mol_type = other DNA
taccaagttaaaaattatttgcacgttagaacc

SEQ ID NO: 102 - DNA, 33 bp, synthetic construct
  source 1..33: mol_type = other DNA
attcgcctgagaaataaagaaattgacttctga

SEQ ID NO: 103 - DNA, 29 bp, synthetic construct
  source 1..29: mol_type = other DNA
tgtttggattatcgtagaaacaataacgg

SEQ ID NO: 104 - DNA, 36 bp, synthetic construct
  source 1..36: mol_type = other DNA
tctttaatttttgcgcgaaccaaaatcggaggcgaa

SEQ ID NO: 105 - DNA, 31 bp, synthetic construct
  source 1..31: mol_type = other DNA
ttcataaccctcagagccaccacaccctcag

SEQ ID NO: 106 - DNA, 32 bp, synthetic construct
  source 1..32: mol_type = other DNA
gtcatagcccccttattagcgtttccagagcc

SEQ ID NO: 107 - DNA, 31 bp, synthetic construct
  source 1..31: mol_type = other DNA
agactgtagcgcgttttcatcggacgtcacc

SEQ ID NO: 108 - DNA, 32 bp, synthetic construct
  source 1..32: mol_type = other DNA
gaattatcagaatcaagtttgcctcgtaatca

SEQ ID NO: 109 - DNA, 31 bp, synthetic construct
  source 1..31: mol_type = other DNA
cggaaattattcacaaacgtagaaaatacat

SEQ ID NO: 110 - DNA, 45 bp, synthetic construct
  source 1..45: mol_type = other DNA
acataaagcaacacattcaaccgatggagggaaggtaaatattga

SEQ ID NO: 111 - DNA, 45 bp, synthetic construct
  source 1..45: mol_type = other DNA
gtgtatcaactcaacccatgtaccgactgagtttcgtcaccagta

SEQ ID NO: 112 - DNA, 31 bp, synthetic construct
  source 1..31: mol_type = other DNA
taattttttcacgttgaaaatcttttgctaa

SEQ ID NO: 113 - DNA, 31 bp, synthetic construct
  source 1..31: mol_type = other DNA
caaactacaacgcaagtatagcccggaatag

SEQ ID NO: 114 - DNA, 32 bp, synthetic construct
  source 1..32: mol_type = other DNA
ttccacagaacaactaaaggaattagtgagaa

SEQ ID NO: 115 - DNA, 18 bp, synthetic construct
  source 1..18: mol_type = other DNA
attagatatcgcaaaatc

SEQ ID NO: 116 - DNA, 32 bp, synthetic construct
  source 1..32: mol_type = other DNA
aaggctccaaaaggagcctttaatgaatttct

SEQ ID NO: 117 - DNA, 45 bp, synthetic construct
  source 1..45: mol_type = other DNA
gcgggcctgctatccaagcttgcatgcaggtcgactctagaggat

SEQ ID NO: 118 - DNA, 31 bp, synthetic construct
  source 1..31: mol_type = other DNA
ccccgggtaccgattgggaagggcgatcggt

SEQ ID NO: 119 - DNA, 31 bp, synthetic construct
  source 1..31: mol_type = other DNA
gggaaacctgtcgtgccagctgcaaagcctg

SEQ ID NO: 120 - DNA, 31 bp, synthetic construct
  source 1..31: mol_type = other DNA
acttttgcgggagagtagatttagtttgacc

SEQ ID NO: 121 - DNA, 31 bp, synthetic construct
  source 1..31: mol_type = other DNA
ggagagggtagctatttttgagatgagaaag

SEQ ID NO: 122 - DNA, 32 bp, synthetic construct
  source 1..32: mol_type = other DNA
atttcaaccgttctagctgataaacaatatga

SEQ ID NO: 123 - DNA, 31 bp, synthetic construct
  source 1..31: mol_type = other DNA
gcaaacataacgccagggttttcaggcgatt

SEQ ID NO: 124 - DNA, 32 bp, synthetic construct
  source 1..32: mol_type = other DNA
aaggctatcaggtcattgcctgagggtaatcg

SEQ ID NO: 125 - DNA, 32 bp, synthetic construct
  source 1..32: mol_type = other DNA
tcgtaatcgcgctcactgcccgcttcacatta

SEQ ID NO: 126 - DNA, 32 bp, synthetic construct
  source 1..32: mol_type = other DNA
atcggccaacgcgcggggagaggctcttttca

SEQ ID NO: 127 - DNA, 31 bp, synthetic construct
  source 1..31: mol_type = other DNA
tattgggtcaatagaaaattcatgtttattt

SEQ ID NO: 128 - DNA, 45 bp, synthetic construct
  source 1..45: mol_type = other DNA
attcccaattctgcgaacgatggcttagagcttttttaattgctg

SEQ ID NO: 129 - DNA, 12 bp, synthetic construct
  source 1..12: mol_type = other DNA
atgcgttatcat

SEQ ID NO: 130 - DNA, 12 bp, synthetic construct
  source 1..12: mol_type = other DNA
ccttagattttc

SEQ ID NO: 131 - DNA, 24 bp, synthetic construct
  source 1..24: mol_type = other DNA
acgctgagctattaattaaatcct

SEQ ID NO: 132 - DNA, 12 bp, synthetic construct
  source 1..12: mol_type = other DNA
cttctgaccttg

SEQ ID NO: 133 - DNA, 36 bp, synthetic construct
  source 1..36: mol_type = other DNA
aatcatagtgagtgaatataaatcgtcgaagagtca

SEQ ID NO: 134 - DNA, 29 bp, synthetic construct
  source 1..29: mol_type = other DNA
tctgacgtttgaaataagttaagattaag

SEQ ID NO: 135 - DNA, 46 bp, synthetic construct
  source 1..46: mol_type = other DNA
atagtgaaattttccgaccgtgtgttaaataagaatattttatcaa

SEQ ID NO: 136 - DNA, 12 bp, synthetic construct
  source 1..12: mol_type = other DNA
ttaatgctaaat

SEQ ID NO: 137 - DNA, 12 bp, synthetic construct
  source 1..12: mol_type = other DNA
taaggcgataaa

SEQ ID NO: 138 - DNA, 46 bp, synthetic construct
  source 1..46: mol_type = other DNA
gactaccttttcaaataaacaccactagaaaaaaacttttttaacc

SEQ ID NO: 139 - DNA, 36 bp, synthetic construct
  source 1..36: mol_type = other DNA
tccggctttttaatggaatcaatatatggtctgaga

SEQ ID NO: 140 - DNA, 12 bp, synthetic construct
  source 1..12: mol_type = other DNA
cataaaacagta

SEQ ID NO: 141 - DNA, 12 bp, synthetic construct
  source 1..12: mol_type = other DNA
catttgcaattt

SEQ ID NO: 142 - DNA, 12 bp, synthetic construct
  source 1..12: mol_type = other DNA
ataattggaatc

SEQ ID NO: 143 - DNA, 13 bp, synthetic construct
  source 1..13: mol_type = other DNA
taacaacgcttaa

SEQ ID NO: 144 - DNA, 53 bp, synthetic construct
  source 1..53: mol_type = other DNA
aatataatgctgtacggtgtctggaagtttttagctatattttcaaattaagc

SEQ ID NO: 145 - DNA, 32 bp, synthetic construct
  source 1..32: mol_type = other DNA
gcgagctgaatatgcaactaaagtagctcaac

SEQ ID NO: 146 - DNA, 31 bp, synthetic construct
  source 1..31: mol_type = other DNA
atattcggcttgctttcgaggttgtatcggt

SEQ ID NO: 147 - DNA, 30 bp, synthetic construct
  source 1..30: mol_type = other DNA
ttatcaaaaaggtggcatcaattttggggc

SEQ ID NO: 148 - DNA, 31 bp, synthetic construct
  source 1..31: mol_type = other DNA
ttatataagagaagcctgtttagtatacaaa

SEQ ID NO: 149 - DNA, 26 bp, synthetic construct
  source 1..26: mol_type = other DNA
acatttaaaattacctttaggttggg

SEQ ID NO: 150 - DNA, 17 bp, synthetic construct
  source 1..17: mol_type = other DNA
cgcctatatgtaaatgc

SEQ ID NO: 151 - DNA, 54 bp, synthetic construct
  source 1..54: mol_type = other DNA
tcaaaaattacgaggcatagtaagtaacgccataacaggattagcagagcgagg

SEQ ID NO: 152 - DNA, 52 bp, synthetic construct
  source 1..52: mol_type = other DNA
gtcagaattagccggaacgaggaccaactttaacaggattagcagagcgagg

SEQ ID NO: 153 - DNA, 54 bp, synthetic construct
  source 1..54: mol_type = other DNA
aaaggaatacaccagaacgagtagatcggccttaacaggattagcagagcgagg

SEQ ID NO: 154 - DNA, 52 bp, synthetic construct
  source 1..52: mol_type = other DNA
tgaaagaattcagtgaataagggccatatttaacaggattagcagagcgagg

SEQ ID NO: 155 - DNA, 56 bp, synthetic construct
  source 1..56: mol_type = other DNA
cctgagtagattttagaactcaaacttaaattggtaacaggattagcagagcgagg

SEQ ID NO: 156 - DNA, 54 bp, synthetic construct
  source 1..54: mol_type = other DNA
ggaatcgtccaaaatagcgagaggcatcagtttaacaggattagcagagcgagg

SEQ ID NO: 157 - DNA, 52 bp, synthetic construct
  source 1..52: mol_type = other DNA
acattatccttatgcgattttattgattagtaacaggattagcagagcgagg

SEQ ID NO: 158 - DNA, 52 bp, synthetic construct
  source 1..52: mol_type = other DNA
aatgttttttgccagagggggtacggaacataacaggattagcagagcgagg

SEQ ID NO: 159 - DNA, 54 bp, synthetic construct
  source 1..54: mol_type = other DNA
ctcattatttaataaaacgaactatttttgggtaacaggattagcagagcgagg

SEQ ID NO: 160 - DNA, 37 bp, synthetic construct
  source 1..37: mol_type = other DNA
aatctacgaccagtctaacaggattagcagagcgagg

SEQ ID NO: 161 - DNA, 54 bp, synthetic construct
  source 1..54: mol_type = other DNA
cgtgaaccgccgtaaagcactaaaggaagaaataacaggattagcagagcgagg

SEQ ID NO: 162 - DNA, 54 bp, synthetic construct
  source 1..54: mol_type = other DNA
aatttaggttttcagaggcatttttgacaagataacaggattagcagagcgagg

SEQ ID NO: 163 - DNA, 52 bp, synthetic construct
  source 1..52: mol_type = other DNA
accggatattcttccaaatcaacgtaacaataacaggattagcagagcgagg

SEQ ID NO: 164 - DNA, 54 bp, synthetic construct
  source 1..54: mol_type = other DNA
ttttcatgtgtgtcgaaatccgcgtacagacctaacaggattagcagagcgagg

SEQ ID NO: 165 - DNA, 54 bp, synthetic construct
  source 1..54: mol_type = other DNA
cgagtaaattttagagtctgtccatataacgttaacaggattagcagagcgagg

SEQ ID NO: 166 - DNA, 52 bp, synthetic construct
  source 1..52: mol_type = other DNA
gctttcctcgttttcagagcgggagctaaataacaggattagcagagcgagg

SEQ ID NO: 167 - DNA, 55 bp, synthetic construct
  source 1..55: mol_type = other DNA
cgcaagacagaatataaagtaccgaaatcaatataacaggattagcagagcgagg

SEQ ID NO: 168 - DNA, 53 bp, synthetic construct
  source 1..53: mol_type = other DNA
tgatgaaattttcaaacatcaagttctgtcctaacaggattagcagagcgagg

SEQ ID NO: 169 - DNA, 56 bp, synthetic construct
  source 1..56: mol_type = other DNA
agacgacgacttttaataaacaacatacaatagataacaggattagcagagcgagg

SEQ ID NO: 170 - DNA, 52 bp, synthetic construct
  source 1..52: mol_type = other DNA
taaccaccgattaaagggatttgtgttttttaacaggattagcagagcgagg

SEQ ID NO: 171 - DNA, 54 bp, synthetic construct
  source 1..54: mol_type = other DNA
gaggcaaaagcgattataccaagccaagaacgtaacaggattagcagagcgagg

SEQ ID NO: 172 - DNA, 52 bp, synthetic construct
  source 1..52: mol_type = other DNA
acgctaacaattttatcctgaaatcgagaataacaggattagcagagcgagg

SEQ ID NO: 173 - DNA, 54 bp, synthetic construct
  source 1..54: mol_type = other DNA
tgccagttatcaagattagttgcttcgtaggataacaggattagcagagcgagg

SEQ ID NO: 174 - DNA, 54 bp, synthetic construct
  source 1..54: mol_type = other DNA
aaatgaaatctaagaacgcgaggcaaggcttataacaggattagcagagcgagg

SEQ ID NO: 175 - DNA, 52 bp, synthetic construct
  source 1..52: mol_type = other DNA
acataaaaaagcgaaaggagcggctgcgcgtaacaggattagcagagcgagg

SEQ ID NO: 176 - DNA, 54 bp, synthetic construct
  source 1..54: mol_type = other DNA
ggtattaatctttccttatcattccgagccagtaacaggattagcagagcgagg

SEQ ID NO: 177 - DNA, 52 bp, synthetic construct
  source 1..52: mol_type = other DNA
caagcaagagcatgtagaaacccaaaaggttaacaggattagcagagcgagg

SEQ ID NO: 178 - DNA, 54 bp, synthetic construct
  source 1..54: mol_type = other DNA
tccggtatacgcgcctgtttatcagttcagcttaacaggattagcagagcgagg

SEQ ID NO: 179 - DNA, 52 bp, synthetic construct
  source 1..52: mol_type = other DNA
agacctgccagaatcctgagaatagacaggtaacaggattagcagagcgagg

SEQ ID NO: 180 - DNA, 51 bp, synthetic construct
  source 1..51: mol_type = other DNA
attgcatatccagaacaatatcttgcccttaacaggattagcagagcgagg

SEQ ID NO: 181 - DNA, 69 bp, synthetic construct
  source 1..69: mol_type = other DNA
gcaaatgaaaacagtttttacaaacaattcgacctacatatcacttgtaacaggattagcagagcgagg

SEQ ID NO: 182 - DNA, 54 bp, synthetic construct
  source 1..54: mol_type = other DNA
gagtccacagccggcgaacgtggcaatgcgcctaacaggattagcagagcgagg

SEQ ID NO: 183 - DNA, 54 bp, synthetic construct
  source 1..54: mol_type = other DNA
atgttttaaattcgagcttcaaagaatgcttttaacaggattagcagagcgagg

SEQ ID NO: 184 - DNA, 54 bp, synthetic construct
  source 1..54: mol_type = other DNA
aaaggccggaagcccgaaagacttaccataaataacaggattagcagagcgagg

SEQ ID NO: 185 - DNA, 52 bp, synthetic construct
  source 1..52: mol_type = other DNA
cagcgaagcaaagcggattgcactattatataacaggattagcagagcgagg

SEQ ID NO: 186 - DNA, 54 bp, synthetic construct
  source 1..54: mol_type = other DNA
gacgagaataagggaaccgaactgcgcagacgtaacaggattagcagagcgagg

SEQ ID NO: 187 - DNA, 54 bp, synthetic construct
  source 1..54: mol_type = other DNA
gtgaattatacaggtagaaagattcttttgcataacaggattagcagagcgagg

SEQ ID NO: 188 - DNA, 54 bp, synthetic construct
  source 1..54: mol_type = other DNA
gttaatatgccagctttcatcaacgcccactataacaggattagcagagcgagg

SEQ ID NO: 189 - DNA, 52 bp, synthetic construct
  source 1..52: mol_type = other DNA
ttgttaatcaaaaataattcgcaatagtaataacaggattagcagagcgagg

SEQ ID NO: 190 - DNA, 54 bp, synthetic construct
  source 1..54: mol_type = other DNA
tttgataatagagagtacctttaaaatactgctaacaggattagcagagcgagg

SEQ ID NO: 191 - DNA, 54 bp, synthetic construct
  source 1..54: mol_type = other DNA
actcctcatggcttttgatgatactacgtaattaacaggattagcagagcgagg

SEQ ID NO: 192 - DNA, 54 bp, synthetic construct
  source 1..54: mol_type = other DNA
agctgctcggacagatgaacggtgacctgctctaacaggattagcagagcgagg

SEQ ID NO: 193 - DNA, 54 bp, synthetic construct
  source 1..54: mol_type = other DNA
acagttaaccttgagtaacagtgctaaagacttaacaggattagcagagcgagg

SEQ ID NO: 194 - DNA, 54 bp, synthetic construct
  source 1..54: mol_type = other DNA
caggaggccacacccgccgcgcttgagaaaggtaacaggattagcagagcgagg

SEQ ID NO: 195 - DNA, 54 bp, synthetic construct
  source 1..54: mol_type = other DNA
ccgccgccctgaatttaccgttccaaacgaaataacaggattagcagagcgagg

SEQ ID NO: 196 - DNA, 52 bp, synthetic construct
  source 1..52: mol_type = other DNA
gtcagacctcattaaagccagatcttaccataacaggattagcagagcgagg

SEQ ID NO: 197 - DNA, 54 bp, synthetic construct
  source 1..54: mol_type = other DNA
aatagctaaagagcaagaaacaatgcctaatttaacaggattagcagagcgagg

SEQ ID NO: 198 - DNA, 54 bp, synthetic construct
  source 1..54: mol_type = other DNA
aaaccgagtgagcgctaatatcagcccaatcctaacaggattagcagagcgagg

SEQ ID NO: 199 - DNA, 54 bp, synthetic construct
  source 1..54: mol_type = other DNA
atcctgttctgaacaccctgaacaaacgtcaataacaggattagcagagcgagg

SEQ ID NO: 200 - DNA, 52 bp, synthetic construct
  source 1..52: mol_type = other DNA
aatccctaacagggaagcgcatagagaatataacaggattagcagagcgagg

SEQ ID NO: 201 - DNA, 54 bp, synthetic construct
  source 1..54: mol_type = other DNA
gcgcatcggataggtcacgttggttggaacaataacaggattagcagagcgagg

SEQ ID NO: 202 - DNA, 54 bp, synthetic construct
  source 1..54: mol_type = other DNA
ctccagccgtaacaacccgtcggaaaagggcgtaacaggattagcagagcgagg

SEQ ID NO: 203 - DNA, 54 bp, synthetic construct
  source 1..54: mol_type = other DNA
aacggtaccaagtgtagcggtcacggcgctagtaacaggattagcagagcgagg

SEQ ID NO: 204 - DNA, 54 bp, synthetic construct
  source 1..54: mol_type = other DNA
taatttacgccgtttttattttcaattttgcataacaggattagcagagcgagg

SEQ ID NO: 205 - DNA, 54 bp, synthetic construct
  source 1..54: mol_type = other DNA
atcggctgaccaagtaccgcactcctcatctttaacaggattagcagagcgagg

SEQ ID NO: 206 - DNA, 69 bp, synthetic construct
  source 1..69: mol_type = other DNA
aaacagttaccctcgtttaccagacgataaaaacataaatattcatttaacaggattagcagagcgagg

SEQ ID NO: 207 - DNA, 64 bp, synthetic construct
  source 1..64: mol_type = other DNA
gagatttataccacattcaactaatgcagatacaagcaacactaacaggattagcagagcgagg

SEQ ID NO: 208 - DNA, 69 bp, synthetic construct
  source 1..69: mol_type = other DNA
gccactacacggagatttgtatcatcgataaataggaagtttccatttaacaggattagcagagcgagg

SEQ ID NO: 209 - DNA, 64 bp, synthetic construct
  source 1..64: mol_type = other DNA
aggcgcattggctgaccttcatcaagagtaatctgcgaaacataacaggattagcagagcgagg

SEQ ID NO: 210 - DNA, 64 bp, synthetic construct
  source 1..64: mol_type = other DNA
gctacagggtactatggttgctttgacgagcacgtcggaacctaacaggattagcagagcgagg

SEQ ID NO: 211 - DNA, 69 bp, synthetic construct
  source 1..69: mol_type = other DNA
aaaaaccggagcccccgatttagagccggggaatattaaagaacgtgtaacaggattagcagagcgagg

SEQ ID NO: 212 - DNA, 69 bp, synthetic construct
  source 1..69: mol_type = other DNA
aaataagaccgacttgcgggaggtttgccttaaacaaaataaacagctaacaggattagcagagcgagg

SEQ ID NO: 213 - DNA, 64 bp, synthetic construct
  source 1..64: mol_type = other DNA
atcattaccccaatagcaagcaaatcagatataggttttagctaacaggattagcagagcgagg

SEQ ID NO: 214 - DNA, 54 bp, synthetic construct
  source 1..54: mol_type = other DNA
gtcgaggtatcacccaaatcaagtgtctggcctaacaggattagcagagcgagg

SEQ ID NO: 215 - DNA, 58 bp, synthetic construct
  source 1..58: mol_type = other DNA
ttttttttttttttttttttataaagctaatggtcaataacctgtcattccatataac

SEQ ID NO: 216 - DNA, 47 bp, synthetic construct
  source 1..47: mol_type = other DNA
ttttttttttttttttttttggttgtaaaacattatgaccctgtaat

SEQ ID NO: 217 - DNA, 38 bp, synthetic construct
  source 1..38: mol_type = other DNA
ttttttttttttttttttttatacaggcaactcagagc

SEQ ID NO: 218 - DNA, 49 bp, synthetic construct
  source 1..49: mol_type = other DNA
ttttttttttttttttttttaataaagcggcaaagaattagcaatctac

SEQ ID NO: 219 - DNA, 61 bp, synthetic construct
  source 1..61: mol_type = other DNA
ttttttttttttttttttttaattttaatagtagtagcattaacatccaataaatcctttt

SEQ ID NO: 220 - DNA, 53 bp, synthetic construct
  source 1..53: mol_type = other DNA
cggagagaaagagcgctcaatttagacggcaaatcaacagttgagagccagca

SEQ ID NO: 221 - DNA, 58 bp, synthetic construct
  source 1..58: mol_type = other DNA
aggacgttgtcacgcaaattaaccgtacattttttcgttgtgtgcgcgggtgtaggat

SEQ ID NO: 222 - DNA, 69 bp, synthetic construct
  source 1..69: mol_type = other DNA
ccgcgcacacaacgtgaaaacatagcgatagcttatttcatctttttgctctttctctccgataagcta

SEQ ID NO: 223 - DNA, 27 bp, synthetic construct
  source 1..27: mol_type = other DNA
atgctcctaatcctacacccgcgcaca

SEQ ID NO: 224 - DNA, 26 bp, synthetic construct
  source 1..26: mol_type = other DNA
gacttagaatagcttatcggagagaa

SEQ ID NO: 225 - DNA, 24 bp, synthetic construct
  source 1..24: mol_type = other DNA
gcgcgggtgtaggattaggagcat

SEQ ID NO: 226 - DNA, 22 bp, synthetic construct
  source 1..22: mol_type = other DNA
ctccgataagctattctaagtc

SEQ ID NO: 227 - DNA, 22 bp, synthetic construct
  misc_binding 22: bound_moiety = 3CholTEG
  source 1..22: mol_type = other DNA
cctcgctctgctaatcctgtta

SEQ ID NO: 228 - DNA, 20 bp, synthetic construct
  misc_binding 20: bound_moiety = 3Cy3Sp; note = 3Cy3Sp
  source 1..20: mol_type = other DNA
aaaaaaaaaaaaaaaaaaaa

SEQ ID NO: 229 - DNA, 20 bp, synthetic construct
  misc_binding 20: bound_moiety = 3Cy5Sp; note = 3Cy5Sp
  source 1..20: mol_type = other DNA
aaaaaaaaaaaaaaaaaaaa

SEQ ID NO: 230 - DNA, 50 bp, synthetic construct
  misc_binding 22: bound_moiety = iCy3; note = iCy3
  source 1..50: mol_type = other DNA
tagaagtacgtctgaaatggattattttgtagcaatacttctagaactgg

SEQ ID NO: 231 - DNA, 69 bp, synthetic construct; identical to SEQ ID NO: 222
  misc_binding 23: bound_moiety = iCy5; note = iCy5
  source 1..69: mol_type = other DNA

SEQ ID NO: 232 - DNA, 53 bp, synthetic construct
  source 1..53: mol_type = other DNA
gtagcattccacagttttgtcgtctttccggaattgcgaataatacgcgaaac

SEQ ID NO: 233 - DNA, 32 bp, synthetic construct
  source 1..32: mol_type = other DNA
acgttgaaagcgtaacgatctaaagacagccc

SEQ ID NO: 234 - DNA, 32 bp, synthetic construct
  source 1..32: mol_type = other DNA
tcatagttgaaccgccaccctcaggagactcc

SEQ ID NO: 235 - DNA, 30 bp, synthetic construct
  source 1..30: mol_type = other DNA
accctcatcttaccgaagccctaaatagca

SEQ ID NO: 236 - DNA, 32 bp, synthetic construct
  source 1..32: mol_type = other DNA
agagagatagagcaagaaacaatgttttaaga

SEQ ID NO: 237 - DNA, 32 bp, synthetic construct
  source 1..32: mol_type = other DNA
aaagtaaggtactcaggaggtttaggggtttt

SEQ ID NO: 238 - DNA, 32 bp, synthetic construct
  source 1..32: mol_type = other DNA
gtatcacccagatagccgaacaaagttaagcc

SEQ ID NO: 239 - DNA, 30 bp, synthetic construct
  misc_feature 1..30: note = NL-core-8
  source 1..30: mol_type = other DNA
atcccaacaaaataaacagccagttaccag

SEQ ID NO: 240 - DNA, 30 bp, synthetic construct
  source 1..30: mol_type = other DNA
aaggaaatataagtatagcccgcgtcgaga

SEQ ID NO: 241 - DNA, 30 bp, synthetic construct
  source 1..30: mol_type = other DNA
agtaagcaggattaggattagcgtaccgcc

SEQ ID NO: 242 - DNA, 42 bp, synthetic construct
  source 1..42: mol_type = other DNA
taagaggctaaccgccaccctcagattttgccaccaccctca

SEQ ID NO: 243 - DNA, 32 bp, synthetic construct
  source 1..32: mol_type = other DNA
tgagccatccaggcggataagtgcgaataggt

SEQ ID NO: 244 - DNA, 47 bp, synthetic construct
  source 1..47: mol_type = other DNA
tcaagagagtcatacatggcttttgaacaggagctgaaacatgaaag

SEQ ID NO: 245 - DNA, 42 bp, synthetic construct
  source 1..42: mol_type = other DNA
attggcctattcacaaacaaataaatcctcattaaccgttcc

SEQ ID NO: 246 - DNA, 32 bp, synthetic construct
  source 1..32: mol_type = other DNA
agccccctttgggaattagagccacaccgact

SEQ ID NO: 247 - DNA, 30 bp, synthetic construct
  source 1..30: mol_type = other DNA
aatcaaaatcattaccagagccaccaccgg

SEQ ID NO: 248 - DNA, 34 bp, synthetic construct
  source 1..34: mol_type = other DNA
gggtaacggacggccagtgccgcgaagagagttg

SEQ ID NO: 249 - DNA, 48 bp, synthetic construct
  source 1..48: mol_type = other DNA
gcctgagtttgattagtaataacacgacgtcgatgtttttaccctaaa

SEQ ID NO: 250 - DNA, 26 bp, synthetic construct
  source 1..26: mol_type = other DNA
ctcgtcaaaggaagcttgcttttatg

SEQ ID NO: 251 - DNA, 32 bp, synthetic construct
  source 1..32: mol_type = other DNA
aaccgcctcaggaggttgaggcagtttaacgg

SEQ ID NO: 252 - DNA, 32 bp, synthetic construct
  source 1..32: mol_type = other DNA
ttttcaggaacactgagtttcgtcgaaccgaa

SEQ ID NO: 253 - DNA, 32 bp, synthetic construct
  source 1..32: mol_type = other DNA
gaattaccagtcaggacgttgggacggaacaa

SEQ ID NO: 254 - DNA, 32 bp, synthetic construct
  source 1..32: mol_type = other DNA
cattattacaacactatcataacctcagaaaa

SEQ ID NO: 255 - DNA, 32 bp, synthetic construct
  source 1..32: mol_type = other DNA
agtaagagcaggtagaaagattcaactggctc

SEQ ID NO: 256 - DNA, 30 bp, synthetic construct
  source 1..30: mol_type = other DNA
ttcatcaccaggcgcataggcttcagttga

SEQ ID NO: 257 - DNA, 30 bp, synthetic construct
  source 1..30: mol_type = other DNA
gatttagcgccaaaaggaattacccgtata

SEQ ID NO: 258 - DNA, 32 bp, synthetic construct
  source 1..32: mol_type = other DNA
atacataagaataccacattcaacatgaacgg

SEQ ID NO: 259 - DNA, 32 bp, synthetic construct
  source 1..32: mol_type = other DNA
tattcattctttgaaagaggacagggaaccca

SEQ ID NO: 260 - DNA, 32 bp, synthetic construct
  source 1..32: mol_type = other DNA
tgtaccgtgatagcaagcccaatattcggaac

SEQ ID NO: 261 - DNA, 32 bp, synthetic construct
  source 1..32: mol_type = other DNA
ggtcagtgaatgccccctgcctattaatgcag

SEQ ID NO: 262 - DNA, 32 bp, synthetic construct
  source 1..32: mol_type = other DNA
accgccacgaaccaccaccagagcaagcaaat

SEQ ID NO: 263 - DNA, 32 bp, synthetic construct
  source 1..32: mol_type = other DNA
atttaaataacaggaagattgtatcgaggcat

SEQ ID NO: 264 - DNA, 57 bp, synthetic construct
  source 1..57: mol_type = other DNA
gtggttccggcgccagggtggtttttcttatcctagtttggaacaagagatccccgg

SEQ ID NO: 265 - DNA, 52 bp, synthetic construct
  source 1..52: mol_type = other DNA
tacgccatcgactctagaggtccactgccccagcaggcgaaattcaccagtg

SEQ ID NO: 266 - DNA, 45 bp, synthetic construct
  source 1..45: mol_type = other DNA
ggaagggccgaattcgtaatcggttgagtgttgttccgtttgatg

SEQ ID NO: 267 - DNA, 47 bp, synthetic construct
  source 1..47: mol_type = other DNA
aagaatcgagatagatggtcatttttagctgtttcctgcaaagcgcc

SEQ ID NO: 268 - DNA, 45 bp, synthetic construct
  source 1..45: mol_type = other DNA
aagaacgggtattaaaccatatcaacaatagttttataagtcctg

SEQ ID NO: 269 - DNA, 53 bp, synthetic construct
  source 1..53: mol_type = other DNA
aacaagaaaaataccaatcaataatcggcatcattaccgcgcccacggtattc

SEQ ID NO: 270 - DNA, 32 bp, synthetic construct
  source 1..32: mol_type = other DNA
caaatcagtacgagcatgtagaaaatatccca

SEQ ID NO: 271 - DNA, 32 bp, synthetic construct
  source 1..32: mol_type = other DNA
tcctaattgccagtaataagagaacatacata

SEQ ID NO: 272 - DNA, 30 bp, synthetic construct
  source 1..30: mol_type = other DNA
ttttcgaaacaccggaatcatacgttaaat

SEQ ID NO: 273 - DNA, 30 bp, synthetic construct
  source 1..30: mol_type = other DNA
gacaaaaaacatataaaagaaacagaggca

SEQ ID NO: 274 - DNA, 42 bp, synthetic construct
  source 1..42: mol_type = other DNA
tagaaaatatataaagtaccgacaattttaaggtaaagtaat

SEQ ID NO: 275 - DNA, 47 bp, synthetic construct
  source 1..47: mol_type = other DNA
aaggtggcgggcgacattcaaccgatggagggacagtatgttagcaa

SEQ ID NO: 276 - DNA, 42 bp, synthetic construct
  source 1..42: mol_type = other DNA
gaaacgtcatgaaaccatcgatagcagcaccgtagcgccaaa

SEQ ID NO: 277 - DNA, 26 bp, synthetic construct
  source 1..26: mol_type = other DNA
actttgctttgaccgttgtttttagc

SEQ ID NO: 278 - DNA, 36 bp, synthetic construct
  source 1..36: mol_type = other DNA
cgtgaacccagggcgcgtaatacttctagaagaact

SEQ ID NO: 279 - DNA, 32 bp, synthetic construct
  source 1..32: mol_type = other DNA
gtagcgcggcaccattaccattagaaattatt

SEQ ID NO: 280 - DNA, 32 bp, synthetic construct
  source 1..32: mol_type = other DNA
tctgtccagcagaacgcgcctgtttcttacca

SEQ ID NO: 281 - DNA, 32 bp, synthetic construct
  source 1..32: mol_type = other DNA
atacccaaccgaggaaacgcaatacctaattt

SEQ ID NO: 282 - DNA, 32 bp, synthetic construct
  source 1..32: mol_type = other DNA
ttaacgtcgagcgtctttccagagaacatgtt

SEQ ID NO: 283 - DNA, 32 bp, synthetic construct
  source 1..32: mol_type = other DNA
cagctaatgacgacgacaataaacaagactcc

SEQ ID NO: 284 - DNA, 32 bp, synthetic construct
  source 1..32: mol_type = other DNA
cattaaagaagaactggcatgattataacgga

SEQ ID NO: 285 - DNA, 38 bp, synthetic construct
  source 1..38: mol_type = other DNA
aaaccaggtgtgaaattgttccggaagcataaaatcaa

SEQ ID NO: 286 - DNA, 34 bp, synthetic construct
  source 1..34: mol_type = other DNA
agaacaatatcacgcaaattaacgagcacccgcc

SEQ ID NO: 287 - DNA, 32 bp, synthetic construct
  source 1..32: mol_type = other DNA
agggaagaaagcgagcgggttaggagtaaaag

SEQ ID NO: 288 - DNA, 26 bp, synthetic construct
  source 1..26: mol_type = other DNA
aaaaacgcagtgaggccaccaatcag

SEQ ID NO: 289 - DNA, 30 bp, synthetic construct
  source 1..30: mol_type = other DNA
tcacacgaccattaaaagggacattctggc

SEQ ID NO: 290 - DNA, 49 bp, synthetic construct
  source 1..49: mol_type = other DNA
aaatggattacgccagaatccggattttagacaggaaagtgagcgtgcc

SEQ ID NO: 291 - DNA, 42 bp, synthetic construct
  source 1..42: mol_type = other DNA
gattgacctgggataggtcacgttggtgtagatgttgttaaa

SEQ ID NO: 292 - DNA, 32 bp, synthetic construct
  source 1..32: mol_type = other DNA
gacagtatgattctccgtgggaaccatcaaaa

SEQ ID NO: 293 - DNA, 30 bp, synthetic construct
  source 1..30: mol_type = other DNA
attcgcataccccggttgataactcgttta

SEQ ID NO: 294 - DNA, 47 bp, synthetic construct
  source 1..47: mol_type = other DNA
atcatatgttaaatttttgttaaatccatttttatcgatgaacggta

SEQ ID NO: 295 - DNA, 42 bp, synthetic construct
  source 1..42: mol_type = other DNA
gtaaaactatagcgagaggcttttgttttcaaaagaagtttt

SEQ ID NO: 296 - DNA, 32 bp, synthetic construct
  source 1..32: mol_type = other DNA
gccagaggtccaatactgcggaataagtacgg

SEQ ID NO: 297 - DNA, 32 bp, synthetic construct
  source 1..32: mol_type = other DNA
ttcatcttcgtgtgataaataaggattactag

SEQ ID NO: 298 - DNA, 32 bp, synthetic construct
  source 1..32: mol_type = other DNA
aaaaagcccaacatgtaatttaggcgcaaaga

SEQ ID NO: 299 - DNA, 32 bp, synthetic construct
  source 1..32: mol_type = other DNA
aacaacgctgtttagtatcatatgggtttgaa

SEQ ID NO: 300 - DNA, 30 bp, synthetic construct
  source 1..30: mol_type = other DNA
gagtcaaagcttagattaagaccgttatac

SEQ ID NO: 301 - DNA, 30 bp, synthetic construct
  source 1..30: mol_type = other DNA
aaattctcttaattgagaatcgtcaacagt

SEQ ID NO: 302 - DNA, 32 bp, synthetic construct
  source 1..32: mol_type = other DNA
cagtagggtaccagtataaagccattgaaaac

SEQ ID NO: 303 - DNA, 32 bp, synthetic construct
  source 1..32: mol_type = other DNA
tctgagagttttcccttagaatccttgtttgg

SEQ ID NO: 304 - DNA, 32 bp, synthetic construct
  source 1..32: mol_type = other DNA
attatactatcaatataatcctgactaaaata

SEQ ID NO: 305 - DNA, 32 bp, synthetic construct
  source 1..32: mol_type = other DNA
ggcaattctctgaataatggaaggcgtcgcta

SEQ ID NO: 306 - DNA, 42 bp, synthetic construct
  source 1..42: mol_type = other DNA
gattagagctatcatcatattcctgttttattatcagatgat

SEQ ID NO: 307 - DNA, 45 bp, synthetic construct
  source 1..45: mol_type = other DNA
ttttacatcgggagaaacagttagaacctacttttcatatcaaaa

SEQ ID NO: 308 - DNA, 53 bp, synthetic construct
  source 1..53: mol_type = other DNA
ttatttgcacgtaggtttaacgtcagatgggcgaattattcatttaacatcaa

SEQ ID NO: 309 - DNA, 32 bp, synthetic construct
  source 1..32: mol_type = other DNA
tgagcaaaattgcgtagattttcaaaacagaa

SEQ ID NO: 310 - DNA, 32 bp, synthetic construct
  source 1..32: mol_type = other DNA
ataaagaaccagaaggagcggaatcgtcaata

SEQ ID NO: 311 - DNA, 30 bp, synthetic construct
  source 1..30: mol_type = other DNA
gaaaccacaaatcaccatcaataaggccgg

SEQ ID NO: 312 - DNA, 32 bp, synthetic construct
  source 1..32: mol_type = other DNA
tatattttattcaaaagggtgagaatgatatt

SEQ ID NO: 313 - DNA, 32 bp, synthetic construct
  source 1..32: mol_type = other DNA
caaccgttaacattatcattttgcagtattag

SEQ ID NO: 314 - DNA, 32 bp, synthetic construct
  source 1..32: mol_type = other DNA
gtttgagtctagctgataaattaataatgtgt

SEQ ID NO: 315 - DNA, 30 bp, synthetic construct
  source 1..30: mol_type = other DNA
tgaccattctgcgaacgagtagtgccggag

SEQ ID NO: 316 - DNA, 30 bp, synthetic construct
  source 1..30: mol_type = other DNA
agggtagcccgaacgttattaagtattaaa

SEQ ID NO: 317 - DNA, 32 bp, synthetic construct
  source 1..32: mol_type = other DNA
ctatcaggctatttttgagagatcacagttga

SEQ ID NO: 318 - DNA, 32 bp, synthetic construct
  source 1..32: mol_type = other DNA
taacctgtagtttcattccatatatttagact

SEQ ID NO: 319 - DNA, 32 bp, synthetic construct
  source 1..32: mol_type = other DNA
ggatagcggggtaatagtaaaatgtggagcaa

SEQ ID NO: 320 - DNA, 30 bp, synthetic construct
  source 1..30: mol_type = other DNA
ccagacgaataaaacgaactaaagaaaaat

SEQ ID NO: 321 - DNA, 32 bp, synthetic construct
  source 1..32: mol_type = other DNA
ttcagaaaacgataaaaaccaaaagcatgtca

SEQ ID NO: 322 - DNA, 32 bp, synthetic construct
  source 1..32: mol_type = other DNA
ggaagcaaacgagaatgaccataattaaacag

SEQ ID NO: 323 - DNA, 53 bp, synthetic construct
  source 1..53: mol_type = other DNA
cccctcaaatgctatcaaaaatcaggtcttcgagcttcaaagcgaattagaga

SEQ ID NO: 324 - DNA, 45 bp, synthetic construct
  source 1..45: mol_type = other DNA
cagaagcaaagcggattgccgtcataaatattttttcattgaatc

SEQ ID NO: 325 - DNA, 32 bp, synthetic construct
  source 1..32: mol_type = other DNA
ataattcgtcattgcctgagagtctacaaagg

SEQ ID NO: 326 - DNA, 32 bp, synthetic construct
  source 1..32: mol_type = other DNA
tttcatcaaacaattcgacaactcttttaaaa

SEQ ID NO: 327 - DNA, 30 bp, synthetic construct
  source 1..30: mol_type = other DNA
cagcagcatttgaggatttagaggaacaaa

SEQ ID NO: 328 - DNA, 32 bp, synthetic construct
  source 1..32: mol_type = other DNA
tcaatatcaattgaggaaggttatacgctcaa

SEQ ID NO: 329 - DNA, 32 bp, synthetic construct
  source 1..32: mol_type = other DNA
atagaaaaaataagtttattttgtccatattt

SEQ ID NO: 330 - DNA, 47 bp, synthetic construct
  source 1..47: mol_type = other DNA
gataatacaaatgaaaaatctaaagcccttgctagcactaacaacta

SEQ ID NO: 331 - DNA, 32 bp, synthetic construct
  source 1..32: mol_type = other DNA
agccagctacattaaatgtgagcgtagccagc

SEQ ID NO: 332 - DNA, 42 bp, synthetic construct
  source 1..42: mol_type = other DNA
acgaaccacagaagataaaacagaggtgaggcggctgagagc

SEQ ID NO: 333 - DNA, 32 bp, synthetic construct
  source 1..32: mol_type = other DNA
caacagagaaacatcgccattaaaaccctcaa

SEQ ID NO: 334 - DNA, 32 bp, synthetic construct
  source 1..32: mol_type = other DNA
cgtaagaattagtctttaatgcgccacaatca

SEQ ID NO: 335 - DNA, 32 bp, synthetic construct
  source 1..32: mol_type = other DNA
caataataaacccacaagaattgagaagcgca

SEQ ID NO: 336 - DNA, 32 bp, synthetic construct
  source 1..32: mol_type = other DNA
acgctaacacaattttatcctgaatattttgc

SEQ ID NO: 337 - DNA, 32 bp, synthetic construct
  source 1..32: mol_type = other DNA
gccagttatccaaataagaaacgagcctttac

SEQ ID NO: 338 - DNA, 38 bp, synthetic construct
  source 1..38: mol_type = other DNA
ttgtatcagaaaggaacaactaaaagacgttagtaaat

SEQ ID NO: 339 - DNA, 32 bp, synthetic construct
  source 1..32: mol_type = other DNA
ctgaccaaacggtcaatcataaggccggaacg

SEQ ID NO: 340 - DNA, 32 bp, synthetic construct
  source 1..32: mol_type = other DNA
tgtacagaagagtaatcttgacaacaaagctg

SEQ ID NO: 341 - DNA, 32 bp, synthetic construct
  source 1..32: mol_type = other DNA
attataccttatgcgattttaagacctgacga

SEQ ID NO: 342 - DNA, 54 bp, synthetic construct
  source 1..54: mol_type = other DNA
gtacctttaattgctccttttgataatatcgcgttttaatttaccctgactatt

SEQ ID NO: 343 - DNA, 30 bp, synthetic construct
  source 1..30: mol_type = other DNA
ctacgttttaatttcaactttatgggcttg

SEQ ID NO: 344 - DNA, 32 bp, synthetic construct
  source 1..32: mol_type = other DNA
ttaattaaccttgcttctgtaaatatatgtga

SEQ ID NO: 345 - DNA, 32 bp, synthetic construct
  source 1..32: mol_type = other DNA
atagcgattagtgaatttatcaaaccggctta

SEQ ID NO: 346 - DNA, 54 bp, synthetic construct
  source 1..54: mol_type = other DNA
taagaacgcgaggcgttttagcgatattttcatcgtaggatgtctttccttatc

SEQ ID NO: 347 - DNA, 30 bp, synthetic construct
  source 1..30: mol_type = other DNA
aagaatattttcaaatatatttgaacgcga

SEQ ID NO: 348 - DNA, 32 bp, synthetic construct
  source 1..32: mol_type = other DNA
ataccgacctgacctaaatttaataaatgctg

SEQ ID NO: 349 - DNA, 54 bp, synthetic construct
  source 1..54: mol_type = other DNA
gaaaacaaaattaattacatttaatacaaaatcgcgcagaaatatacagtaaca

SEQ ID NO: 350 - DNA, 32 bp, synthetic construct
  source 1..32: mol_type = other DNA
tgtctggatttaaatatgcaactactgtagct

SEQ ID NO: 351 - DNA, 32 bp, synthetic construct
  source 1..32: mol_type = other DNA
ttcccaattagatacatttcgcaattggggcg

SEQ ID NO: 352 - DNA, 32 bp, synthetic construct
  source 1..32: mol_type = other DNA
aggtaaagaaatgcaatgcctgagtctactaa

SEQ ID NO: 353 - DNA, 30 bp, synthetic construct
  source 1..30: mol_type = other DNA
agacagtgataaaaatttttagttatttca

SEQ ID NO: 354 - DNA, 20 bp, synthetic construct
  source 1..20: mol_type = other DNA
ttgccctcgcctggccctga

SEQ ID NO: 355 - DNA, 24 bp, synthetic construct
  source 1..24: mol_type = other DNA
aaagcactaaatcggaggggtcga

SEQ ID NO: 356 - DNA, 37 bp, synthetic construct
  source 1..37: mol_type = other DNA
gggagcccccgatatcacccaaatcaagtgcccacta

SEQ ID NO: 357 - DNA, 41 bp, synthetic construct
  source 1..41: mol_type = other DNA
gcgcttttttaatgcgccgctattagagcttgacgaagccg

SEQ ID NO: 358 - DNA, 46 bp, synthetic construct
  source 1..46: mol_type = other DNA
gaggcggcgtattggaaatcggttttcaaaatcccttatgcgggga

SEQ ID NO: 359 - DNA, 28 bp, synthetic construct
  source 1..28: mol_type = other DNA
cagcattttagcggtccacgctagctga

SEQ ID NO: 360 - DNA, 37 bp, synthetic construct
  source 1..37: mol_type = other DNA
agctgcattaatgaactggggtgcctaatgcacacaa

SEQ ID NO: 361 - DNA, 22 bp, synthetic construct
  source 1..22: mol_type = other DNA
aagtgtaaagctcggccaacgc

SEQ ID NO: 362 - DNA, 45 bp, synthetic construct
  source 1..45: mol_type = other DNA
cgggcgctacactgccctccagtcgggaaacctgtctaactcaca

SEQ ID NO: 363 - DNA, 33 bp, synthetic construct
  source 1..33: mol_type = other DNA
agcgggagctgggcgctggcaagtgtaaaggag

SEQ ID NO: 364 - DNA, 33 bp, synthetic construct
  source 1..33: mol_type = other DNA
gctttcctctcacgctgcgcgtaacagaaagga

SEQ ID NO: 365 - DNA, 29 bp, synthetic construct
  source 1..29: mol_type = other DNA
gcgaacgtggcgcaccacacgtataacgt

SEQ ID NO: 366 - DNA, 36 bp, synthetic construct
  source 1..36: mol_type = other DNA
ttaattgcttttgttgcgctaaacaggagattaaag

SEQ ID NO: 367 - DNA, 31 bp, synthetic construct
  source 1..31: mol_type = other DNA
gaaaactatatagaaggcttatcatagcaag

SEQ ID NO: 368 - DNA, 32 bp, synthetic construct
  source 1..32: mol_type = other DNA
atgcaaatccaatcgcaagacaaatagttaat

SEQ ID NO: 369 - DNA, 31 bp, synthetic construct
  source 1..31: mol_type = other DNA
ggttgggttatataactatatgtgctgagaa

SEQ ID NO: 370 - DNA, 32 bp, synthetic construct
  source 1..32: mol_type = other DNA
gtgaataaactacctttttaacctatcatagg

SEQ ID NO: 371 - DNA, 31 bp, synthetic construct
  source 1..31: mol_type = other DNA
tacataaatcaatataacggattcgcctgat

SEQ ID NO: 372 - DNA, 45 bp, synthetic construct
  source 1..45: mol_type = other DNA
tgctttgacaagtcaatttcatttgaccttttttaatggaaacag

SEQ ID NO: 373 - DNA, 45 bp, synthetic construct
  source 1..45: mol_type = other DNA
aacaagcagttttacctcccgacttgaggttttgaagccttaaat

SEQ ID NO: 374 - DNA, 31 bp, synthetic construct
  source 1..31: mol_type = other DNA
agagagaataacataaaaacaggtattattt

SEQ ID NO: 375 - DNA, 31 bp, synthetic construct
  source 1..31: mol_type = other DNA
caagattagttgcagtaccgcactcatcgag

SEQ ID NO: 376 - DNA, 32 bp, synthetic construct
  source 1..32: mol_type = other DNA
acccagctaaaaatgaaaatagcattttttgt

SEQ ID NO: 377 - DNA, 49 bp, synthetic construct
  source 1..49: mol_type = other DNA
cgaaggggcaaaagaatacacttgataaagtcgaaatccgcgacctgct

SEQ ID NO: 378 - DNA, 39 bp, synthetic construct
  source 1..39: mol_type = other DNA
tcagcggagaatatcgccaaaacactcatctttgaacgg

SEQ ID NO: 379 - DNA, 32 bp, synthetic construct
  source 1..32: mol_type = other DNA
ttagacgggagaattaactgaacactaatatc

SEQ ID NO: 380 - DNA, 32 bp, synthetic construct
  source 1..32: mol_type = other DNA
aggagcctaatctccaaaaaaaagattttttc

SEQ ID NO: 381 - DNA, 45 bp, synthetic construct
  source 1..45: mol_type = other DNA
aagcccgacttcaaagaggtcatttggatggcttagagcttaatt

SEQ ID NO: 382 - DNA, 31 bp, synthetic construct
  source 1..31: mol_type = other DNA
gctgaatataatgatcaaaaagattaagagg

SEQ ID NO: 383 - DNA, 31 bp, synthetic construct
  source 1..31: mol_type = other DNA
cgagctgaaaaggtggcatcaatatttagtt

SEQ ID NO: 384 - DNA, 31 bp, synthetic construct
  source 1..31: mol_type = other DNA
ccatgttacttagaaacaactttcaacagtt

SEQ ID NO: 385 - DNA, 31 bp, synthetic construct
  source 1..31: mol_type = other DNA
ctcattcagtgaataaggcttgcggctgacc

SEQ ID NO: 386 - DNA, 32 bp, synthetic construct
  source 1..32: mol_type = other DNA
aggcgcagacccaaatcaacgtaagaaccgga

SEQ ID NO: 387 - DNA, 31 bp, synthetic construct
  source 1..31: mol_type = other DNA
agatggtactccaacaggtcaggaccagacc

SEQ ID NO: 388 - DNA, 32 bp, synthetic construct
  source 1..32: mol_type = other DNA
gaaacaccagaacgagtagtaaatatcattgt

SEQ ID NO: 389 - DNA, 32 bp, synthetic construct
  source 1..32: mol_type = other DNA
caacatgtttagctatattttcatatggtcaa

SEQ ID NO: 390 - DNA, 32 bp, synthetic construct
  source 1..32: mol_type = other DNA
tagtagtagcattgggagaagcctaaccctca

SEQ ID NO: 391 - DNA, 31 bp, synthetic construct
  source 1..31: mol_type = other DNA
acgcaagagaagatgatgaaacacaattacc

SEQ ID NO: 392 - DNA, 44 bp, synthetic construct
  source 1..44: mol_type = other DNA
tatattcggtcgctacaacagaggtgaatttcttaagctccaaa

SEQ ID NO: 393 - DNA, 29 bp, synthetic construct
  source 1..29: mol_type = other DNA
cgcttttgtcatgaggaaccgatagttgc

SEQ ID NO: 394 - DNA, 36 bp, synthetic construct
  source 1..36: mol_type = other DNA
gaaagacagcggctacagaggctttgaatgccacta

SEQ ID NO: 395 - DNA, 45 bp, synthetic construct
  source 1..45: mol_type = other DNA
ttctgtatgggattttgctaccagtacaaactttttacaacgcct

SEQ ID NO: 396 - DNA, 54 bp, synthetic construct
  source 1..54: mol_type = other DNA
gctcagtacgcagtctctgaatttaagccagataacaggattagcagagcgagg

SEQ ID NO: 397 - DNA, 52 bp, synthetic construct
  source 1..52: mol_type = other DNA
gggttgagtgaattatcaccgtgcaaaatctaacaggattagcagagcgagg

SEQ ID NO: 398 - DNA, 54 bp, synthetic construct
  source 1..54: mol_type = other DNA
atggaaagtattagcgtttgccatattaagtttaacaggattagcagagcgagg

SEQ ID NO: 399 - DNA, 52 bp, synthetic construct
  source 1..52: mol_type = other DNA
accagtattttcatcggcatttcatcactttaacaggattagcagagcgagg

SEQ ID NO: 400 - DNA, 56 bp, synthetic construct
  source 1..56: mol_type = other DNA
aagggggatgtttttgctgcaaggcgcttttcattaacaggattagcagagcgagg

SEQ ID NO: 401 - DNA, 54 bp, synthetic construct
  source 1..54: mol_type = other DNA
ctattatttgtactggtaataagtgtcagacgtaacaggattagcagagcgagg

SEQ ID NO: 402 - DNA, 52 bp, synthetic construct
  source 1..52: mol_type = other DNA
gcattgaccctcagagccgccattcgctattaacaggattagcagagcgagg

SEQ ID NO: 403 - DNA, 52 bp, synthetic construct
  source 1..52: mol_type = other DNA
aacagttccttgagtaacagtgcgccgccataacaggattagcagagcgagg

SEQ ID NO: 404 - DNA, 54 bp, synthetic construct
  source 1..54: mol_type = other DNA
cgccaccacctcagagccaccaccaactgttgtaacaggattagcagagcgagg

SEQ ID NO: 405 - DNA, 54 bp, synthetic construct
  source 1..54: mol_type = other DNA
gccccaaatgtaaacgttaatattctcagagctaacaggattagcagagcgagg

SEQ ID NO: 406 - DNA, 56 bp, synthetic construct
  source 1..56: mol_type = other DNA
gtaccgagctgatcggtgcgggcctcccctcagataacaggattagcagagcgagg

SEQ ID NO: 407 - DNA, 54 bp, synthetic construct
  source 1..54: mol_type = other DNA
caaactatttttcggccttgctggatcagtagtaacaggattagcagagcgagg

SEQ ID NO: 408 - DNA, 52 bp, synthetic construct
  source 1..52: mol_type = other DNA
cgacagaatcattgcctttagcgtcagacttaacaggattagcagagcgagg

SEQ ID NO: 409 - DNA, 54 bp, synthetic construct
  source 1..54: mol_type = other DNA
ttattacgaggtaaatattgacggcaaggccgtaacaggattagcagagcgagg

SEQ ID NO: 410 - DNA, 54 bp, synthetic construct
  source 1..54: mol_type = other DNA
attcgccattttttcaggctgcgcggcgcatctaacaggattagcagagcgagg

SEQ ID NO: 411 - DNA, 52 bp, synthetic construct
  source 1..52: mol_type = other DNA
gtaaccgtgcattcagtttgaggggacgactaacaggattagcagagcgagg

SEQ ID NO: 412 - DNA, 55 bp, synthetic construct
  source 1..55: mol_type = other DNA
agtctgtccattaccgccagccattcctgaaagtaacaggattagcagagcgagg

SEQ ID NO: 413 - DNA, 53 bp, synthetic construct
  source 1..53: mol_type = other DNA
tgagaagtttttgtttttataatctcatggataacaggattagcagagcgagg

SEQ ID NO: 414 - DNA, 56 bp, synthetic construct
  source 1..56: mol_type = other DNA
aatacctacattttttttgacgctcattcaccagtaacaggattagcagagcgagg

SEQ ID NO: 415 - DNA, 52 bp, synthetic construct
  source 1..52: mol_type = other DNA
cccgtcgcggcctcaggaagatggtgccggtaacaggattagcagagcgagg

SEQ ID NO: 416 - DNA, 54 bp, synthetic construct
  source 1..54: mol_type = other DNA
acaagagataaccaataggaacgcaaacggcgtaacaggattagcagagcgagg

SEQ ID NO: 417 - DNA, 54 bp, synthetic construct
  source 1..54: mol_type = other DNA
caccacggttcatatggtttaccatatttttgtaacaggattagcagagcgagg

SEQ ID NO: 418 - DNA, 52 bp, synthetic construct
  source 1..52: mol_type = other DNA
tgaaaggtggtcagttggcaaagaactgattaacaggattagcagagcgagg

SEQ ID NO: 419 - DNA, 54 bp, synthetic construct
  source 1..54: mol_type = other DNA
tctttagggaacctcaaatatcaaaataccgataacaggattagcagagcgagg

SEQ ID NO: 420 - DNA, 54 bp, synthetic construct
  source 1..54: mol_type = other DNA
actttacactgcaacagtgccacgtcagtatttaacaggattagcagagcgagg

SEQ ID NO: 421 - DNA, 52 bp, synthetic construct
  source 1..52: mol_type = other DNA
tcctttgcgtctggccttcctgagtaacaataacaggattagcagagcgagg

SEQ ID NO: 422 - DNA, 54 bp, synthetic construct
  source 1..54: mol_type = other DNA
aatggctatacgtggcacagacaataatatcctaacaggattagcagagcgagg

SEQ ID NO: 423 - DNA, 52 bp, synthetic construct
  source 1..52: mol_type = other DNA
agccctaatagaacccttctgagcaacaggtaacaggattagcagagcgagg

SEQ ID NO: 424 - DNA, 54 bp, synthetic construct
  source 1..54: mol_type = other DNA
aacaccgctatttacattggcagaatcgtctgtaacaggattagcagagcgagg

SEQ ID NO: 425 - DNA, 78 bp, synthetic construct
  source 1..78: mol_type = other DNA
ggtgccgtaaaccgtctatcagggtgtaaaacccagggttttcccagttcggtcattaacaggattagcagagcgagg

SEQ ID NO: 426 - DNA, 69 bp, synthetic construct
  source 1..69: mol_type = other DNA
agacgggcaacggtttattaaagaacgtggacctgcagggctggcgataacaggattagcagagcgagg

SEQ ID NO: 427 - DNA, 72 bp, synthetic construct
  source 1..72: mol_type = other DNA
catacgagatccgctcacaattccggttccggcaccgcttctcgcactcctaacaggattagcagagcgagg

SEQ ID NO: 428 - DNA, 54 bp, synthetic construct
  source 1..54: mol_type = other DNA
tcatagttgaaccgccaccctcaggagactcctaacaggattagcagagcgagg

SEQ ID NO: 429 - DNA, 54 bp, synthetic construct
  source 1..54: mol_type = other DNA
aaagtaaggtactcaggaggtttaggggtttttaacaggattagcagagcgagg

SEQ ID NO: 430 - DNA, 52 bp, synthetic construct
  source 1..52: mol_type = other DNA
aaggaaatataagtatagcccgcgtcgagataacaggattagcagagcgagg

SEQ ID NO: 431 - DNA, 54 bp, synthetic construct
  source 1..54: mol_type = other DNA
agccccctttgggaattagagccacaccgacttaacaggattagcagagcgagg

SEQ ID NO: 432 - DNA, 54 bp, synthetic construct
  source 1..54: mol_type = other DNA
aaccgcctcaggaggttgaggcagtttaacggtaacaggattagcagagcgagg

SEQ ID NO: 433 - DNA, 54 bp, synthetic construct
  source 1..54: mol_type = other DNA
cattattacaacactatcataacctcagaaaataacaggattagcagagcgagg

SEQ ID NO: 434 - DNA, 52 bp, synthetic construct
  source 1..52: mol_type = other DNA
gatttagcgccaaaaggaattacccgtatataacaggattagcagagcgagg

SEQ ID NO: 435 - DNA, 54 bp, synthetic construct
  source 1..54: mol_type = other DNA
tgtaccgtgatagcaagcccaatattcggaactaacaggattagcagagcgagg

SEQ ID NO: 436 - DNA, 54 bp, synthetic construct
  source 1..54: mol_type = other DNA
accgccacgaaccaccaccagagcaagcaaattaacaggattagcagagcgagg

SEQ ID NO: 437 - DNA, 54 bp, synthetic construct
  source 1..54: mol_type = other DNA
tcctaattgccagtaataagagaacatacatataacaggattagcagagcgagg

SEQ ID NO: 438 - DNA, 54 bp, synthetic construct
  source 1..54: mol_type = other DNA
gtagcgcggcaccattaccattagaaattatttaacaggattagcagagcgagg

SEQ ID NO: 439 - DNA, 54 bp, synthetic construct
  source 1..54: mol_type = other DNA
cagctaatgacgacgacaataaacaagactcctaacaggattagcagagcgagg

SEQ ID NO: 440 - DNA, 54 bp, synthetic construct
  source 1..54: mol_type = other DNA
gacagtatgattctccgtgggaaccatcaaaataacaggattagcagagcgagg

SEQ ID NO: 441 - DNA, 54 bp, synthetic construct
  source 1..54: mol_type = other DNA
aaaaagcccaacatgtaatttaggcgcaaagataacaggattagcagagcgagg

SEQ ID NO: 442 - DNA, 52 bp, synthetic construct
  source 1..52: mol_type = other DNA
aaattctcttaattgagaatcgtcaacagttaacaggattagcagagcgagg

SEQ ID NO: 443 - DNA, 54 bp, synthetic construct
  source 1..54: mol_type = other DNA
attatactatcaatataatcctgactaaaatataacaggattagcagagcgagg

SEQ ID NO: 444 - DNA, 52 bp, synthetic construct
  source 1..52: mol_type = other DNA
gaaaccacaaatcaccatcaataaggccggtaacaggattagcagagcgagg

SEQ ID NO: 445 - DNA, 54 bp, synthetic construct
  source 1..54: mol_type = other DNA
caaccgttaacattatcattttgcagtattagtaacaggattagcagagcgagg

SEQ ID NO: 446 - DNA, 52 bp, synthetic construct
  source 1..52: mol_type = other DNA
agggtagcccgaacgttattaagtattaaataacaggattagcagagcgagg

SEQ ID NO: 447 - DNA, 54 bp, synthetic construct
  source 1..54: mol_type = other DNA
ggatagcggggtaatagtaaaatgtggagcaataacaggattagcagagcgagg

SEQ ID NO: 448 - DNA, 54 bp, synthetic construct
  source 1..54: mol_type = other DNA
ttcagaaaacgataaaaaccaaaagcatgtcataacaggattagcagagcgagg

SEQ ID NO: 449 - DNA, 54 bp, synthetic construct
  source 1..54: mol_type = other DNA
agccagctacattaaatgtgagcgtagccagctaacaggattagcagagcgagg

SEQ ID NO: 450 - DNA, 54 bp, synthetic construct
  source 1..54: mol_type = other DNA
caacagagaaacatcgccattaaaaccctcaataacaggattagcagagcgagg

SEQ ID NO: 451 - DNA, 54 bp, synthetic construct
  source 1..54: mol_type = other DNA
cgtaagaattagtctttaatgcgccacaatcataacaggattagcagagcgagg

SEQ ID NO: 452 - DNA, 69 bp, synthetic construct
  source 1..69: mol_type = other DNA
tcaagagagtcatacatggcttttgaacaggagctgaaacatgaaagtaacaggattagcagagcgagg

SEQ ID NO: 453 - DNA, 64 bp, synthetic construct
  source 1..64: mol_type = other DNA
attggcctattcacaaacaaataaatcctcattaaccgttcctaacaggattagcagagcgagg

SEQ ID NO: 454 - DNA, 69 bp, synthetic construct
  source 1..69: mol_type = other DNA
aaggtggcgggcgacattcaaccgatggagggacagtatgttagcaataacaggattagcagagcgagg

SEQ ID NO: 455 - DNA, 64 bp, synthetic construct
  source 1..64: mol_type = other DNA
gaaacgtcatgaaaccatcgatagcagcaccgtagcgccaaataacaggattagcagagcgagg

SEQ ID NO: 456 - DNA, 64 bp, synthetic construct
  source 1..64: mol_type = other DNA
gattgacctgggataggtcacgttggtgtagatgttgttaaataacaggattagcagagcgagg

SEQ ID NO: 457 - DNA, 69 bp, synthetic construct
  source 1..69: mol_type = other DNA
atcatatgttaaatttttgttaaatccatttttatcgatgaacggtataacaggattagcagagcgagg

SEQ ID NO: 458 - DNA, 69 bp, synthetic construct
  source 1..69: mol_type = other DNA
gataatacaaatgaaaaatctaaagcccttgctagcactaacaactataacaggattagcagagcgagg

SEQ ID NO: 459 - DNA, 64 bp, synthetic construct
  source 1..64: mol_type = other DNA
acgaaccacagaagataaaacagaggtgaggcggctgagagctaacaggattagcagagcgagg

SEQ ID NO: 460 - DNA, 53 bp, synthetic construct
  source 1..53: mol_type = other DNA
gccgacaatggaggcttgcagggagttaaaggctttttttttttttttttttt

SEQ ID NO: 461 - DNA, 35 bp, synthetic construct
  source 1..35: mol_type = other DNA
tgctttcaccatcgctttttttttttttttttttt

SEQ ID NO: 462 - DNA, 64 bp, synthetic construct
  source 1..64: mol_type = other DNA
atagctagagggtaattgagcgccctgaacaaagtcattaattgtttttttttttttttttttt

SEQ ID NO: 463 - DNA, 78 bp, synthetic construct
  source 1..78: mol_type = other DNA
aaagtacacagcgattataccaagacagcttgatagtttccattaaccccacggagattttttttttttttttttttt

SEQ ID NO: 464 - DNA, 69 bp, synthetic construct
  source 1..69: mol_type = other DNA
gtaaaatacgtaggactaaagacttttcgggatcgtcaccctcagcagctttttttttttttttttttt

(19) United States
(12) Patent Application Publication
(10) Pub. No.: US 2025/0051830 A1
(43) Pub. Date: Feb. 13, 2025

(54) MEMBRANE-EMBEDDED NANOPORE AND USES THEREOF

(71) Applicant: ARIZONA BOARD OF REGENTS ON BEHALF OF ARIZONA STATE UNIVERSITY, Scottsdale, AZ (US)

(72) Inventors: Hao YAN, Gilbert, AZ (US); Swarup DEY, Middleton, WI (US); Fei ZHENG, Union, NJ (US)

(21) Appl. No.: 18/700,676

(22) PCT Filed: Oct. 12, 2022
(86) PCT No.: PCT/US2022/077962

§ 371 Date: Apr. 11, 2024

Related U.S. Application Data
(60) Provisional application No. 63/255,867, filed on Oct. 14, 2021.

(51) Int. Cl. C12Q 1/6825; C12Q 1/6818

(52) U.S. Cl. CPC C12Q 1/6825; C12Q 1/6818

(57) ABSTRACT

A nanostructure comprising: a) a base plate sheet comprising a single-duplex layer comprised of DNA origami configured in a planar arrangement, b) a pore configured within the center of the base plate sheet, and c) a multi-duplex layer-thick wall comprising a single-duplex layer comprised of DNA origami that extends downward from the base plate sheet on the periphery of the pore, wherein the base plate sheet comprises a single-stranded DNA region on the bottom surface for lipid membrane anchor modification.

### RELATED APPLICATIONS AND INCORPORATION BY REFERENCE

This application claims the benefit of U.S. Provisional application No. 63/255,867, filed Oct. 14, 2021, which is herein incorporated by reference in its entirety.

### FEDERAL FUNDING LEGEND

This invention was made with government support under 1644745 awarded by National Science Foundation. The government has certain rights in the invention.

### SEQUENCE STATEMENT

The instant application contains a Sequence Listing, which has been submitted electronically and is hereby incorporated by reference in its entirety. Said ASCII copy, created Oct. 9, 2022, is named G1118-03201 Corrected Sequence listing.xml and is 414 kb in size.

### FIELD OF THE INVENTION

The instant application relates to a wide membrane-embedded nanopore, designed via DNA nanotechnology, which can readily insert into lipid layers. The nanopore may further comprise a lid to reversibly block and unblock the pore.

### BACKGROUND OF THE INVENTION

The following includes information that may be useful in understanding the present invention. It is not an admission that any of the information, publications or documents specifically or implicitly referenced herein is prior art, or essential, to the presently described or claimed inventions.

Controlled molecular transport across cellular membranes is a ligand-gated event by which channels selectively enable the transport of a variety of small molecules and proteins into a cell. The channels perform a wide range of roles such as in signal transduction and amplification, and import of nutrients. While varied, most channels regulate transport of ions and small molecules by binding a ligand, which causes a nanomechanical change to alter the channels' transport properties, until the ligand dissociates.

### SUMMARY OF THE INVENTION

The inventions described and claimed herein have many attributes and aspects including, but not limited to, those set forth or described or referenced in this Summary. It is not intended to be all-inclusive and the inventions described and claimed herein are not limited to or by the features or embodiments identified in this Summary, which is included for purposes of illustration only and not restriction.

This disclosure provides for a DNA nanostructure comprising i) a base plate sheet that comprises a single-duplex layer DNA origami plate configured in a planar arrangement and ii) a pore configured within the center of the base plate sheet, wherein the base plate sheet comprises a single-stranded DNA region on the bottom surface for lipid membrane anchor modification. The base plate forming helices run horizontally, i.e., about perpendicular to the pore axis, enabling generation of a nanopore with a large diameter central pore and a large surface area available for cholesterol modification, and further enabling pore diameter modulation.

In some aspects, the nanostructure of the present application may be reversely gated by having a lid that can block and unblock the central pore, by e.g., a key and reverse key mechanism, to control the transmembrane flux of cargoes.

In some aspects, this disclosure provides a nanostructure that may comprise:
a) a base plate sheet that may comprise a single-duplex layer comprised of DNA origami configured in a planar arrangement, and
b) a pore that may be configured within the center of the base plate sheet, wherein the base plate sheet may comprise a single-stranded DNA region on the bottom surface for lipid membrane anchor modification.

The nanostructure of the disclosure may further comprise a multi-duplex layer-thick wall that may comprise a single-duplex layer comprised of DNA origami and extend downward from the base plate sheet on the periphery of the pore. The multi-duplex layer-thick wall may be four-duplex layers-thick. The multi-duplex layer-thick wall may be configured perpendicular to the base plate sheet.

The nanostructure of the disclosure may further comprise a lipid membrane anchor covalently linked to a single-stranded DNA sequence which is partially complementary to the single-stranded DNA region of the base plate sheet. The lipid membrane anchor may be selected from cholesterol, or a fatty alcohol. In some aspects, the fatty alcohol is a linear C₆-C₂₂ alkyl chain alcohol. In some aspects, the fatty alcohol may be selected from tetradecanol, hexadecanol, or octadecanol. In some aspects, the lipid membrane anchor may be cholesterol.

The nanostructure of the disclosure may further comprise a lid that may be configured to block the pore in a closed position, and one or a plurality of flexible hinge sequences that may connect the lid to the base plate sheet.

The hinge sequences may each independently comprise a single-stranded region. In certain embodiments, the hinge sequences may each independently comprise a 4-nucleotide single stranded region.

The lid may comprise a single-duplex layer comprised of DNA origami. The lid may further comprise one or a plurality of a first half-lock, wherein each first half-lock independently comprises a single-stranded DNA sequence. The base plate sheet may comprise one or a plurality of a second half-lock that may comprise a single-stranded DNA sequence, wherein the first half-lock and the second half-lock may be partially complementary to each other. In certain embodiments, the lid may comprise two first half-locks, and the base plate may comprise two second half-locks.

The nanostructure of the disclosure may further comprise a single-stranded key DNA sequence which may be partially complementary to the regions of complementarity between the first half-lock and the second half-lock. The purpose of the key is to compete with the base plate- or lid-connected complement so as to disrupt the base plate/lid hybridization allowing the lid to open.

The nanostructure of the disclosure may further comprise a reverse key single-stranded DNA sequence that may be partially complementary to the regions of complementarity between the single-stranded key DNA sequence and the first or second half-lock.

When the nanostructure does not comprise the key single-stranded DNA sequence or the reverse-key single-stranded DNA sequence, the lid may be in a closed position such that the pore is blocked. When the nanostructure comprise the key single-stranded DNA sequence but no reverse-key single-stranded DNA sequence, the lid may be in an open position such that the pore is not blocked. When the nanostructure comprise both the key single-stranded DNA sequence and the reverse-key single-stranded DNA sequence, the lid may be closing such that the pore is partially blocked, or alternatively in a closed position such that the pore is blocked.

One of the first half-lock and the second half-lock may comprise one half of a FRET pair, and the other may comprise the other half of a FRET pair. In some aspects, the first half-lock comprises a donor fluorphore and the second half-lock comprises an acceptor fluorophore or quencher. When the first- and second-half locks are opened, the FRET pair is in a different configuration such that the fluorescence is changed (which can include a different wavelength, different fluorescence intensity, or different fluorescent lifetime).

The base plate sheet may comprise an index region that may comprise a single-duplex layer comprised of DNA origami and a single-stranded DNA region for fluorophore modification. The nanostructure may further comprise a fluorophore covalently linked to a single-stranded DNA sequence which is partially complementary to the single-stranded DNA region in the index region, for e.g., nanostructure tracing.

In some aspects, the base plate sheet may be rectangular or square shaped and have a width or length ranging from 65 to 78 nm. In some aspects, the baseplate is square shaped and has a size of about 70 nm×70 nm. In some aspects, the pore may be square or rectangular shaped and have a width or length ranging from 18 to 24 nm. In some aspects, the pore is square shaped and has a size of about 20 nm×20 nm.

In certain embodiments, the nanostructure of the disclosure comprising a lid may be prepared from a scaffold strand and staple strands having sequences set forth in SEQ ID NOs: 1-150 and optionally SEQ ID NOs: 220-222. In certain embodiments, the nanostructure of the disclosure comprising a lid and a region for lipid membrane anchor modification may be prepared by using a scaffold strand and staple strands having sequences set forth in SEQ ID NOs: 1-214 and optionally SEQ ID NOs: 220-222. In certain embodiments, the nanostructure of the disclosure comprising a lid, a region for lipid membrane anchor modification and a region for fluorophore modification may be prepared by using a scaffold strand and staple strands set forth in SEQ ID NOs: 1-219 and optionally SEQ ID NOs: 220-222. In certain embodiments, the nanostructure of the disclosure comprising a lid and a lipid membrane anchor may be prepared by using a scaffold strand and staple strands set forth in SEQ ID NOs: 1-214, 227, optionally SEQ ID NOs: 220-222. In certain embodiments, the nanostructure of the disclosure comprising a lid, a lipid membrane anchor and a fluorophore may be prepared by using a scaffold strand and staple strands set forth in SEQ ID NOs: 1-219, 227, 228, and optionally SEQ ID NOs: 220-222, or alternatively SEQ ID NOs: 1-219, 227, 229, and optionally SEQ ID NOs:220-222. In certain embodiments, the first half-lock and the second half-lock may comprise the nucleotide sequence of SEQ ID NOs: 222 and 220, respectively, and the key single-strand DNA sequence and the reverse key single-stranded DNA sequences may be set forth in SEQ ID NOs: 224 and 226, respectively. In certain embodiments, the first half-lock and the second half-lock may comprise the nucleotide sequence of SEQ ID NO: 222 and 221, respectively, and the key single-strand DNA sequence and the reverse key single-stranded DNA sequences may be set forth in SEQ ID NOs: 223 and 225, respectively. In certain embodiments, the nanostructure of the disclosure comprising a lid, and a FRET (fluorescence resonance energy transfer) fluorophore pair may be prepared by using a scaffold strand and staple strands set forth in SEQ ID NOs: 1-150, 220, 221, 230 and 231. The scaffold strand may be a M13 genome nucleotide strand, or a M13 genome-derived nucleotide strand. In certain embodiments, the scaffold strand is M13mp18.

In some aspects, the nanostructure of the disclosure comprising no lid or region for lipid membrane anchor modification may be prepared by using a scaffold strand and staple strands set forth in SEQ ID NOs: 232-395. In certain embodiments, the nanostructure of the disclosure comprising a region for lipid membrane anchor modification but no lid may be prepared by using a scaffold strand and staple strands set forth in SEQ ID NOs: 232-459. In certain embodiments, the nanostructure of the disclosure comprising a region for lipid membrane anchor modification and a region for fluorophore modification but no lid may be prepared by using a scaffold strand and staple strands set forth in SEQ ID NOs: 232-464. The scaffold strand may be a M13 genome nucleotide strand, or a M13 genome-derived nucleotide strand. In certain embodiments, the scaffold strand is M13mp18.

In some aspects, the disclosure provides a composition comprising the nanostructure of the disclosure and a lipid bilayer. The lipid bilayer may be part of a cell membrane, or a unilamellar vesicle membrane.

The disclosure also provides a composition comprising the nanostructure of the disclosure and a semi-fluid membrane formed of polymers. The polymer forming the semi-fluid membrane may be composed of amphiphilic synthetic block copolymers. The amphiphilic synthetic block copolymer may be composed of hydrophilic copolymer blocks and hydrophobic copolymer blocks.

In some aspects, the disclosure provides a method of delivering an agent through a lipid bilayer.

In some aspects, the method of the disclosure may comprise:
a) contacting an agent and a nanostructure of the disclosure without a lid with a lipid bilayer, wherein the lipid membrane anchor forms a complex with the lipid bilayer, and
b) allowing the agent to traverse through the pore through the lipid bilayer.

The lipid bilayer may be part of a cell or a unilamellar vesicle.

The method may further comprise applying a voltage across the lipid bilayer and the agent may be an ionic molecule.

The agent may be selected from a small hydrophobic molecule, an ion, or a folded protein. The small hydrophobic molecule may be a fluorophore. The small hydrophobic molecule may have a molecular weight of less than 1000 Da. The folded protein may comprise a hydrodynamic radius of less than the pore size.

The method may further comprise providing an impermeable agent which does not traverse through the pore. The folded protein can be unfolded and the unfolded protein has a hydrodynamic radius greater than the pore size such that it is impermeable through the pore.

In some aspects, the method of the disclosure may comprise:
a) contacting an agent and a lidded nanostructure of the disclosure having a pore in a closed configuration with a lipid bilayer, wherein the lipid membrane anchor forms a complex with the lipid bilayer,
b) presenting a single-stranded key DNA sequence to the nanostructure, whereby the lid opens and the pore is in an open configuration, and
c) allowing the agent to traverse through the pore through the lipid bilayer.

The lipid bilayer may be part of a cell membrane.

The method may comprise applying a voltage across the lipid bilayer, and the agent may be an ionic molecule.

The agent may be selected from a small hydrophobic molecule, an ion, or a folded protein. The small hydrophobic molecule may be a fluorophore. The small hydrophobic molecule may be a molecular weight of less than 1000 Da. The folded protein may comprise a hydrodynamic radius of less than the pore size.

The method may further comprise providing an impermeable agent which does not traverse through the pore when the pore is in an open configuration. The folded protein can be unfolded and the unfolded protein has a hydrodynamic radius greater than the pore size such that it is impermeable through the pore.

The disclosure also provides the use of the nanostructures described herein for high sensitivity bioassays, creation of a synthetic cell or artificial cell networks, and delivery of therapeutic agents such as therapeutic proteins.

In some aspects, the disclosure provides a method of in vivo delivering a therapeutic agent to a required site, comprising administering a key single-stranded DNA sequence modified to bind a target protein at the required site, and later a unilamellar vesicle which is inserted with a nanostructure of the disclosure and filled with a therapeutic agent.

The therapeutic agent may be a folded protein, e.g., having a hydrodynamic radius of less than the pore size. The key single-stranded DNA sequence may be modified to bind a cell surface protein, e.g., a tumor associated antigen.

The vesicle may be a small unilamellar vesicle or a giate unilamellar vesicle.

The vesicle filled with a therapeutic agent may be prepared by:
a) contacting a therapeutic agent and a lidded nanostructure of the disclosure having a pore in a closed configuration with the vesicle, wherein the lipid membrane anchor forms a complex with the lipid bilayer of the vesicle,
b) presenting a key single-stranded DNA sequence to the nanostructure, wherein the lid opens and the pore is in an open configuration,
c) allowing the agent to traverse through the pore through the lipid bilayer, and
d) presenting a reverse key single-stranded DNA sequence to the nanostructure, wherein the lid closes.

Other features and advantages of the instant disclosure will be apparent from the following detailed description and examples which should not be construed as limiting. The contents of all references, GenBank entries, patents and published patent applications cited throughout this application are expressly incorporated herein by reference.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1: Design novelty of Large Diameter Nanopore. (a) Side view (left) and top view (right) of the nanopore of Diederichs et al, Nat Comm, 2019, exemplifying routing of helices in reference DNA nanopores. The helical axis of the component helices ran vertically in these DNA nanopores, i.e.—parallel to the vertical axis of the nanopore. (b-i) Side view of the large and gated DNA channel (LGC) of the present application, comprising M13 scaffold, plate forming staples and pore forming staples. The pore forming helices run horizontally, i.e.—perpendicular to the pore axis. This horizontal routing ensures fine tuning of the pore diameter and a large surface area of the plate, available for cholesterol placement. (b-ii) Zoomed-in image of the pore region showing layered crossover design to form the plate and the pore of LGC, routed in mutually normal planes (horizontal plane (upper in b-ii)—plate forming helices, vertical plane (lower in b-ii)—pore forming helices). The distance between two interhelical crossovers determines their relative spatial alignment. Hence, interhelical crossovers were created so as to be spaced apart in a multiple of 0.5 turns, i.e. —1×0.5 turns=6 nt; 2×0.5 turns=10.5 nt; 3×0.5 turns=16 nt etc. for the helices on the same plane (i.e., —among plate forming helices in the ‘plate plane’ and among pore forming helices in the ‘pore plane’). Whereas the gap between a crossover in the ‘plate plane’ and a crossover in the vertical ‘pore plane’ is kept as an odd multiple of 0.25 turns, except 1×0.25 turns i.e. —3×0.25 turns=8 nt; 5×0.25 turns=13 nt; 7×0.25 turns=18 nt etc. This ensures the ‘plate plane’ and the ‘pore plane’ to be perpendicular to each other. (b-iii) The direction of the helices at crossover points. Circle represents top view of a helix and arrows represent tangent drawn to a helix at a given point to show its direction, horizontal arrows—helices in plate plane and vertical arrows—helices in pore plane. Top panel—relative angle between the helical direction at two crossover points in the plate plane. Bottom panel—relative angle between the direction of a helix at a crossover in the plate plane and a crossover in the pore plane. (c-i) Top view of the pore. (c-ii) Zoomed-in view of the corners where a 4 base loops in only one of the two strands in each helix imposes a 90° curvature in the same plane. (c-iii) Zoom in view of a corner crossover showing the 4 base loop insertion strategy.

FIG. 2: A rationally designed large and gated channel (LGC). a-b, Structural model of the large and gated channel (LGC) containing a 20.4 nm×20.4 nm-wide channel lumen into a 70 nm×70 nm single-duplex layer plate. Hydrophobic cholesterol anchors, placed around the pore on the bottom surface of the plate helps the nanopore to insert through the bilayer. A lid can be reversibly closed (a-i) and opened (a-ii), by a key and reverse key mechanism to control the transmembrane flux of cargoes. b, Top view of LGC. c, key and reverse key mechanism. Two locks formed by the hybridization of two sets of complementary strands, placed on the lid and on the plate initially keeps the lid closed. Addition of the key (s) displaces the locks and thereby opens the lid. Addition of the reverse key(s) displaces the key as a key-reverse key complex, leading to reclosing of the lock back to the initial state. d, AFM images of cholesterol-free LGC without lid (d-i), with closed lid (d-ii) and with opened lid (d-iii). Scale bars 50 nm.

FIG. 3: Position of hydrophobic cholesterol anchors and optional fluorophore modification in the large diameter nanopore. Handle strands of specific sequence are extended from the 64 denoted locations (current cholesterol positions), ensuring the downward orientation of the helices at the location. When cholesterol bearing anti-handle strands are added, their binding at the handle strand bearing locations tag the nanopore with 64 cholesterol molecules that help its insertion into the lipid bilayer. Although, this work only uses the denoted locations, the horizontally routed flat design offers more place that can accommodate at least 96 more cholesterol molecules (possible cholesterol positions) for future design of larger nanopore structures. The fluorophores are placed in the index region to ensure sufficient distance from the functional regions of the nanopore to avoid any undesired non-specific interactions.

FIG. 4: (a) Side view and (b) top view of the optimally designed flexible hinge of the LGC lid formed by a 4-nucleotide single stranded region in each hinge. Several designs with rigid hinge (data not shown) did not lead to successful closure of the lid, perhaps because of strain in the hinge region.

FIG. 5: Details of the strand displacement reaction for reversible lid opening and closing. Lid and plate are locked in two positions, each lock containing a strand with a toehold. Opening key opens the lock by toehold mediated strand displacement (TMSD), leading to the open lid. The opening key also contains a toehold which then initiates a second TMSD when closing keys are added. As a result, the opening key dissociates from the lock strands and lock strands reseal to form the closed lid.

FIG. 6: (a) AFM images and (b) mean structure and root mean squared fluctuations (RMSF) obtained from oxDNA simulations for non-cholesterol versions of—(i) LGC without lid (LGC-N), (ii) LGC with closed lid (LGC-C) and (iii) LGC with open lid (LGC-O). AFM scale bars: 50 nm.

FIG. 7: 1.5% Agarose gel electrophoresis characterization of the formation of the large diameter nanopores (LGC). (a) No lid-LGC with cholesterol (LGC-N+chol) and without cholesterol (LGC-N). Slight upshift of the LGC-N with respect to the scaffold M13 shows its correct formation with a predominant monomer band and a slight dimer band. The successful cholesterol modification is shown by upshift of monomer and dimer bands of LGC-N+Chol compared to the LGC-N band and increased smearing due to aggregation in case of LGC-N+Chol. (b) Formation of non-cholesterol LGC with lid (LGC-C/O) in 1.5% agarose gel. The predominant band of LGC-C/O with lid upshifts compared to that for LGC-N which indicates successful formation of the lid.

FIG. 8: TEM characterization of non-cholesterol LGC. (a) Without lid. (b) With closed lid. (c) With open lid. The edge of the nanopore is annotated with dashed line for ease of understanding. The pore in the middle of the square is visible in all cases. The pore is empty in case of LGC without lid (a). A lid is clearly visible in the middle of the pore in case of LGC with closed lid (b) and open lid (c). In case of LGC with closed lid in (b), the lid looks flat on the pore covering it whereas in case of LGC with open lid in (c), the lid looks slightly tilted owing to its open form. Scale bar: 50 nm.

FIG. 9: Insertion and interaction of LGC with lipid bilayer. a, 1.5% agarose gel analysis of the DNA pore and its binding activity with lipid membrane. Lanes from left to right: 1 kb DNA ladder (from bottom 1 kb, 3 kb and 10 kb respectively), no lid LGC without cholesterol modifications (LGC-N-Chol), incubated with SUVs (DOPC/DOPE=7:3, 5 mM total lipid), no lid LGC with 64 cholesterol modifications (LGC-N+Chol) incubated with SUVs (DOPC/DOPE=7:3, total lipid concentration 0, 0.01, 0.025, 0.05, 0.25 and 0.5 mM, respectively). b, Representative TEM images of LGC-N+Chol pores bound to lipid vesicles. The arrowheads pinpoint the pores. Scale bars=50 nm. c, GUV dye-influx assay scheme (c-i) and their respective time series confocal images (c-ii) at the given intervals with Cy3-labelled LGC and atto-633 dye. Top: LGC-N+Chol readily interacts with bilayer (circle around GUVs) and their insertion leads to influx of the atto-633 dye inside the GUV interior. Bottom: LGC-N-Chol does not interact with bilayer (no circle around GUV) or insert into the GUV, showing no dye influx over the course of 3 hours. d, Bar plot showing percentage of GUVs showing a filled interior after 3 hours. Data shows average percentage of influx and error bars show standard deviation of mean percentage influx counted from n=156 GUVs in case of LGC-N+Chol and n=124 for LGC-N-Chol. e, Example electrophysiological trace showing a single LGC-N+Chol inserted into a planar DPhPC membrane. An applied voltage of +80 mV is shown for the first 5 s of the trace, after which the voltage potential is switched to −80 mV. f, Conductance histogram of 15 individual LGC-N+Chol insertions obtained at 20 mV. g, Current-voltage (IV) trace showing average current of 15 individual insertions±SEM at membrane potentials ranging from −100 mV to +100 mV in 20 mV steps. All electrophysiological experiments conducted in buffers composed of 1M KCl, 10 mM HEPES pH 7.6.

FIG. 10: Large field of view TEM images showing interaction of no lid LGC (LGC-N) with lipid membrane. LGC-N structures are marked with arrows. Scale bars: 100 nm. (a) POPC-SUVs+LGC-N with cholesterol TEM images show most LGC-N structures on top of the lipid membranes as they strongly interact with the lipid membrane. (b) POPC-SUVs+LGC-N without cholesterol TEM images show most LGC structures away from the SUVs due to lack of interaction between unmodified LGC-N structures and lipid membrane.

FIG. 11: (a) Scheme and (b) Transmitted light and Cy3 channel merged images of GUV influx assay in FIG. 9c-ii, showing that the GUVs remained on focus across the imaging. Top: no lid LGC with cholesterol (LGC-N+Chol), bottom: no lid LGC without cholesterol (LGC-N-chol). Scale bar: 10 μm.

FIG. 12: Reversible gating by LGC lid dynamically controls cargo transport across lipid bilayer. a-i, Scheme showing lidded LGC, containing donor dye on plate and acceptor dye on the lid. Addition of key and reverse key leads to respectively, opening and closing of the lid—resulting in furthering or nearing of the dye pairs. a-ii, Fluorescence spectra (D and A on graph represent fluorescence maxima for donor and acceptor respectively) and a-iii, FRET efficiency corresponding to reversible gating of LGC lid; data points and error bars respectively represent average relative FRET efficiency and standard deviation of mean from n=3 experiments. Initially when lid is closed, spatial proximity of the donor and acceptor enables FRET, resulting in lower donor fluorescence, higher acceptor fluorescence (a-ii, solid line with higher peak) and high FRET efficiency (a-iii, closed). Opening the lid using key (dynamic-open) moves the donor and acceptors far from each other. Thus, FRET ceases and results in higher donor fluorescence, lower acceptor fluorescence (a-ii, lower solid line) and lower FRET efficiency (a-iii, Dyn. Open). Closing the lid back from its open state using reverse key (dynamic-closed) restores FRET, again resulting in lower donor fluorescence, higher acceptor fluorescence (a-ii, dashed line) and high FRET efficiency (a-iii, Dyn. Closed). b, GUV dye-influx assay scheme (b-i) and their respective time series confocal images (b-ii) at the given intervals with cholesterol and Cy3-labelled LGC and atto-633 dye. In all cases the LGC inserts through the bilayer. Top—closed lid does not allow atto-633 dye influx into the GUV interior. Middle—dynamically opening the LGC lid with key results in influx of atto-633 dye into the GUV (the time starts from the last frame of the closed lid LGC). Bottom—separate experiment with dynamic-closed LGC added to GUVs does not lead to any dye influx, showing dynamic closing is effective. c, Example electrophysiological trace showing a single LGC-C inserted into a planar DPhPC membrane. An applied voltage of +80 mV is shown for the first 5 s of the trace, after which the voltage potential is switched to −80 mV. d, Current-voltage (IV) trace showing average current of 13 individual insertions±standard error of mean of LGC-C at membrane potentials ranging from −100 mV to +100 mV in 20 mV steps. e, Conductance histogram of 13 individual insertions of LGC-C obtained at 20 mV. Inset shows example trace of transition from closed-lid pore to open-lid after addition of 15 nM opening key. f, Example electrophysiological trace showing a single LGC-O inserted into a planar DPhPC membrane. An applied voltage of +80 mV is shown for the first 5 s of the trace, after which the voltage potential is switched to −80 mV. g, Current-voltage (IV) trace showing average current of 17 individual insertions±standard error of mean of LGC-O at membrane potentials ranging from −100 mV to +100 mV in 20 mV steps. h, Conductance histogram of 17 individual insertions of LGC-O obtained at 20 mV. Inset shows example trace of transition from open-lid pore to closed-lid after addition of 15 nM closing key. All electrophysiological experiments conducted in buffers composed of 1M KCl, 10 mM HEPES pH 7.6.

FIG. 13: (a-i) FRET spectra and (a-ii) corresponding FRET efficiencies—for opening of LGC with closed lid by mismatch key and correct key. FRET spectra and FRET efficiencies of the LGC with closed lid with two different sets of mismatch keys (FIG. 13(a-i), dark grey and light grey) behave similar to closed lid structure only, i.e.—low donor fluorescence at λₘₐₓ^{Cy3}=564 nm and higher acceptor fluorescence at λₘₐₓ^{Cy5}=670 nm (a-i; black, light grey and dark grey curves) and higher FRET efficiency, a-ii; black, dark grey and light grey bars). Whereas the same with correct opening key leads to opening of the lid and hence shows higher donor fluorescence at λₘₐₓ^{Cy3}=564 nm and lower acceptor fluorescence at λₘₐₓ^{Cy5}=670 nm (a-i) and higher FRET efficiency (a-ii). The FRET efficiency bars and error bars in a-ii represents mean relative FRET efficiency and standard deviation of the mean respectively, obtained from three technical replicates.

FIG. 14: Kinetics of lid opening. Data is fitted with second order kinetic rate equation (R²=0.94995) to obtain Rate constant=1940±50 M⁻¹ S⁻¹.

FIG. 15: (a) Scheme and (b) Transmitted light and Cy3 channel merged images of GUV influx assay showing LGC binding to membrane and that the GUVs remained in focus during the imaging with cholesterol modified LGC structures in FIG. 12b-ii. Top: LGC, closed lid; middle panels—LDN, dynamically opened lid; bottom panels—LGC, dynamically closed lid from the open state. Scale bar: 10 μm.

FIG. 16: Negative control experiments for FIG. 12b-ii. (a) Cy3 labelled LGC structures without cholesterol modification do not bind to the GUV membrane. (b) Unmodified LGC with closed lid (top panels) or with opened lid (bottom panels) does not show any influx of atto-633 dye into GUVs. Scale bar-10 μm.

FIG. 17: (a-i) Scheme and (a-ii) confocal images of Atto 633 influx in GUVs showing—top: Cy3 and cholesterol labelled LGC with closed lid inserts in GUV but prevents Atto 633 influx. Bottom—addition of mismatch key does not open the LGC lid and thus no Atto-633 influx is observed unlike that in the case of correct opening key in FIG. 12b-ii. (b-i) Scheme and (b-ii) confocal images of GFP influx in GUVs showing—top: Cy5 and cholesterol labelled LGC with closed lid inserts in GUV but prevents GFP influx. Bottom—addition of mismatch key does not open the LGC lid and thus no GFP influx is observed unlike that in the case of correct opening key in FIG. 22a-ii. Scale bar 10 μm.

FIG. 18: All point histogram analysis of (a) LGC-closed and (b) LGC-open at positive (right) and negative (left) 20, 40, 60, 80 and 100 mV.

FIG. 19: (a) Dynamic closing of open DNA pores at 10 mV (a-i), 50 mV (a-ii) and −50 mV (a-iii). Closing DNA strands added at a concentration of 15 nM to the buffer solution at 37° C. (b) Dynamic opening of closed DNA pores at −10 mV (b-i), −20 mV (b-ii) and −50 mV (b-iii). Opening DNA strands added at a concentration of 15 nM to the buffer solution at 37° C.

FIG. 20: (a) GUV dye-influx assay scheme and (b) their respective time series confocal images at the given intervals with Cy5-labelled LGC-N and GFP. Top: Cholesterol modified LGC-N (LGC-N+Chol) readily interacts with bilayer (circle around GUVs) and their insertion leads to influx of the atto-633 dye inside the GUV interior. Bottom: LGC without cholesterol modification (LGC-N-chol) does not interact with bilayer (no circle around GUV) or insert into the GUV, showing no dye influx over the course of 3 hours. Bar plot showing percentage of GUVs showing a filled interior after 3 hours. Data shows average % influx and error bars show standard deviation of mean counted from n=75 GUVs in case of LGC+chol and n=124 for LGC.

FIG. 21: (a) Scheme and (b) Transmitted light & Cy5 channel merged images of GUV influx assay with cholesterol modified LGC structures FIG. 22a-ii, showing that the GUVs remained in focus during the imaging. Top: LGC, closed lid; Middle: LGC, dynamically opened lid; Bottom: LGC, dynamically closed lid from the open state. Scale bars 10 μm.

FIG. 22: Reversibly gated Protein transport by large diameter nanopore. a, GUV GFP-influx assay scheme (a-i), their respective time series confocal images at the given intervals with Cy5-labelled LGC and GFP (a-ii) and the corresponding rate of percentage of GUVs showing a filled interior over time (a-iii) (Data shows average percentage of influx and error bars show standard deviation of mean counted from n=125, 255 and 125 GUVs for Closed, Dyn. Open and Dyn. Closed versions of LGC respectively). Cholesterol modified LGC inserts into GUV bilayer in all cases. Top: LGC with closed lid restricts influx of GFP into the GUV interior. Middle: upon addition of the key, the lid of the LGC dynamically opens and results in influx of GFP into the GUV (the time series starts from the last frame of the closed lid LGC). Bottom—separate experiment with dynamic-closed LGC added to GUVs does not lead to any GFP influx, showing dynamic closing is effective in stopping the flux of GFP. b-c, Electrophysiological characterization of controlled Trypsin transport. i, Scheme, ii, example electrophysiological trace and iii, all-point conductance histogram of a single LGC at −50 mV after the addition of 38 μM trypsin to the cis chamber in case of LGC-C (b) and LGC-N (c). All electrophysiological experiments conducted in buffers composed of 1M KCl, 10 mM HEPES pH 7.6.

FIG. 23: Negative control experiments for FIG. 22a-ii. (a) Cy5 labelled LGC structures without cholesterol modification do not bind to the GUV membrane. (b) GUV influx assay with non-cholesterol LGC, no lid (top row), with closed lid (middle row) or with opened lid (bottom row) does not show any influx of GFP into GUVs. Scale bar 10 μm.

FIG. 24: Size-selective transport through nanopore. Cy3 and cholesterol labelled nanopore without lid inserts through the bilayer but a large molecule such as 500 KDa FITC-Dextran (green) cannot pass through the nanopore. Scale bar 10 μm.

FIG. 25: Trypsin translocation analysis comparing dwell time against percentage pore block. Two types of block occur, a small block (type I, top dots) associated with interaction at the nanopore lumen entrance without translocation and a larger block (type II, lower dots) associated with full translocation of trypsin through the nanopore.

### DETAILED DESCRIPTION OF THE INVENTION

Using natural cellular transport channels beyond their biogenic remit can be difficult because of their often fragile protein architectures, narrow size-range for cargo, limited choice of triggers, and reduced control when a channel switches back to its original state. While previous attempts to build programmable synthetic channels for defined transport of large biorelevant cargo into cells, their reports have identified numerous challenges such that there is no commercialized synthetic pore which selectively enables the transport of molecules into a cell. Previous attempts at designing synthesis cellular membrance channels has only resulted in barrel-like and constitutively open protein pores of a few nm in width (Mahendran, K. R. et al. A monodisperse transmembrane a-helical peptide barrel. Nat. Chem. 9, 411-419, (2016); Xu, C. et al. Computational design of transmembrane pores. Nature 585, 129-134, (2020)). For example, Diederichs, T. et al. designed a DNA nanopore whose duplex helices are routed ‘vertically’, i.e., at 90 degree, relative to the membrane plane (FIG. TA) (Diederichs, T. et al. Synthetic protein-conductive membrane nanopores built with DNA. Nat. Commun. 10, 5018, 1-11, (2019)). Such an arrangement places the majority of DNA into an extra-membrane cap region with multi-duplex layer-thick channel walls while restricting the amount of DNA available for forming a wide membrane-embedded pore. The small lateral footprint of the pore limits the number of attachment points for lipid anchors required for efficient pore insertion into bilayer membranes.

Fragasso A et al., recently produced a rigid octagonal ring-like nanopore with ‘horizontal’ routing, consisted of four single-duplex layer DNA origami plates, with 32 cholesterols attached to the outer side of the pore (Fragasso A, De Franceschi N, Stommer P, van der Sluis E O, Dietz H, Dekker C. Reconstitution of Ultrawide DNA Origami Pores in Liposomes for Transmembrane Transport of Macromolecules [published online ahead of print, 2021 Jun. 25]. ACS Nano. 15(8), 12768-12779, (2021)). Such a design enables the fabrication of large pores having an inner diameter as large as 30 nm. However, the authors reported it was nearly impossible to embed these wide pores into lipid bilayers, suggesting they would not likely embed into cell walls in vivo.

One representative nanostructure of this disclosure can be made via DNA nanotechnology design principles and features an about 416 nm²-wide opening and a nanomechanical lid that can be controllably closed and re-opened via a lock-and-key mechanism, which is all beyond nature's functional remit. Compared to previous construction routes, de novo design with DNA nanotechnology (Douglas, S. M. et al. Self-assembly of DNA into nanoscale three-dimensional shapes. Nature 459, 414-418, (2009); Seeman, N. C. & Sleiman, H. F. DNA nanotechnology. Nat. Rev. Mater. 3, 1-23, (2017); Dey, S. et al. DNA origami. Nat. Rev. Methods Primers 1, 1-24, (2021); Ke, Y., Ong, L. L., Shih, W. M. & Yin, P. Three-dimensional structures self-assembled from DNA bricks. Science 338, 1177, (2012)). offers unprecedented structural precision and tenability, dynamic-nanomechanical control, a wide range of chemical modifications, and stability in harsh conditions. Rational design with DNA has previously led to structurally simple membrane-spanning DNA nanopores with inner width of a few nm (Langecker, M. et al. Synthetic lipid membrane channels formed by designed DNA nanostructures. Science 338, 932-936, (2012); J. R., Stulz, E. & Howorka, S. Self-assembled DNA nanopores that span lipid bilayers. Nano Lett. 13, 2351-2356, (2013); List, J., Weber, M. & Simmel, F. C. Hydrophobic actuation of a DNA origami bilayer structure. Angew. Chem. Int. Ed. 53, 4236-4239, (2014); Burns, J. R., Seifert, A., Fertig, N. & Howorka, S. A biomimetic DNA-based channel for the ligand-controlled transport of charged molecular cargo across a biological membrane. Nat. Nanotechnol. 11, 152-156, (2016); Göpfrich, K. et al. Ion channels made from a single membrane-spanning DNA duplex. Nano Lett. 16, 4665-4669, (2016); Göpfrich, K. et al. Large-conductance transmembrane porin made from DNA origami. ACS Nano 10, 8207-8214, (2016); Krishnan, S. et al. Molecular transport through large-diameter DNA nanopores. Nat. Commun. 7, 12787, (2016); Diederichs, T. et al. Synthetic protein-conductive membrane nanopores built with DNA. Nat. Commun. 10, 5018, 1-11, (2019); Thomsen, R. P. et al. A large size-selective DNA nanopore with sensing applications. Nat. Commun. 10, 5655, (2019); Lanphere, C., Arnott, P. M., Jones, S. F., Korlova, K. & Howorka, S. A biomimetic DNA—based membrane gate for protein—controlled transport of cytotoxic drugs. Angew. Chem. 133, 1931-1936, (2020)). The creation of the synthetic channel of the disclosure explores the wider scope of DNA nanotechnology to integrate binding of artificial ligands, triggered nanomechanical changes for opening and closing, and transport of nanoscale large cargo across membranes.

The DNA nanostructure's features include: sequence specific fully reversible gating within the pore; a reconfigurable nanomechanical lid which allows for control of cargo of various sizes; a pore size of more than 5.8 nm the maximum dimensions of any natural analogue; reversible control of flow of cargo which allows for protein capture while interacting with free-flowing ligands; which reversible protein capture allows for visualization of real time protein folding and unfolding; an about 416 nm² wide opening and a nanomechanical lid which can be controllably closed and reopened via a lock and key mechanism; the ability to span across lipid bilayers, including lipid bilayers of a cell membrane; the use of scaffold DNA to create structures in a modular manner; and the inclusion, in some aspects, of a reversibly ligand gated lid-controlled transport of small molecule cargo.

The reconfigurable nanomechanical lid enables control over transport of various cargoes ranging from small molecule dye to folded proteins of up to 5.8 nm hydrodynamic diameter. This exceeds the natural analogue-ligand gated ion channels which have much narrower pore dimension. The DNA nanopore with a cross-section of about 416 nm² which is significantly higher than biological and previously reported DNA nanopores (Fragasso A, De Franceschi N, Stommer P, van der Sluis E O, Dietz H, Dekker C. Reconstitution of Ultrawide DNA Origami Pores in Liposomes for Transmembrane Transport of Macromolecules [published online ahead of print, 2021 Jun. 25]. ACS Nano. 15(8), 12768-12779, (2021)). The design freedom offered by the DNA nanostructures of this disclosure enables creation of pores to reversibly capture proteins and study their interaction with free-flowing ligands or real-time protein folding and unfolding. The reversibly controlled flow of cargo by a nanomechanical lid may also be exploited to transport bioactive cargo into cells, or to construct cell-cell communication for artificial gap junctions and to enable integration of artificial tissues with living cells and tissues for tissue engineering. In summary, the DNA na****nostructures of this disclosure extend the versatility and scope of artificial nanopores beyond nature and thus opens up various exciting applications, including for use as a.

The functional DNA device may be useful in highly sensitive biosensing, drug delivery of proteins, and the creation of artificial cell networks.

Unless otherwise indicated, the practice of the present invention employs techniques of chemistry, molecular biology, microbiology, recombinant DNA technology, and chemical methods, which are within the capabilities of a person of ordinary skill in the art. Such techniques are also explained in the literature, for example, M. R. Green, J. Sambrook, 2012, Molecular Cloning: A Laboratory Manual, Fourth Edition, Books 1-3, Cold Spring Harbor Laboratory Press, Cold Spring Harbor, N.Y.; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press.

### Definitions

The term “DNA nanotechnology” refers to a branch of nanotechnology concerned with the design, study and application of DNA-based synthetic structures to take advantage of the physical and chemical properties of DNA.

The term “DNA origami” refers a kind of DNA technology that is for bottom-up fabrication of well-defined nanostructures ranging from tens of nanometers to sub-micrometers. Specifically, it means the nanoscale folding of DNA to create two- and three-dimensional shapes at the nanoscale. The specificity of the interactions between complementary base pairs make DNA a useful construction material, through design of its base sequences.

The “scaffold strand” used in DNA origami technique refers to a long single stranded DNA, typically a viral genome of about 7,000 nucleotides long, while the ‘staple strand’ refers to the short single stranded DNA. Each staple strand has multiple binding domains that bind and accordingly bring together otherwise distant regions of the scaffold strand via crossover base pairing, folding the scaffold strand in a manner similar to knitting. The resulting DNA nanostructure as formed totally depends on the scaffold strand and staple strands as selected.

The term “duplex” as used herein refers to a double-stranded DNA, formed by bonding and coiling the nucleotides of two complimentary DNA sequences to form a double helix.

The term “nanostructure” as used herein refers to a predesigned two or three dimensional molecular structure typically comprised from a biopolymer, suitably a naturally or non-naturally occurring nucleic acid, which structure has at least one dimension or an aspect of its geometry that is within the nanoscale (i.e., 10-meters). Nanoscale structures suitably have dimensions or geometry of less than around 100 nm, typically less than 50 nm, and most suitably less than 20 nm. Nanoscale structures suitably possess dimensions or geometry greater than around 0.1 nm, typically greater than around 1 nm, and optionally greater than around 2 nm. Assembly of nucleic acid nanostructures may occur spontaneously in solution, or may require presence of additional co-factors including, but not limited to, nucleic acid scaffolds, nucleic acid aptamers, nucleic acid staples, co-enzymes, and molecular chaperones. Where desired nanostructures result from one or more predesigned spontaneously self-folding nucleic acid molecules, such as DNA, this is typically referred to as nucleic acid ‘origami’. Exemplary three dimensional DNA nanostructures may comprise nanobarrels; nanorafts, which are typically rectangular, polygonal, circular, or ellipsoid substantially planar nanostructures; nanospheres and regular or irregular polyhedral nanostructures, including stellated polyhedral nanostructures. Exemplary two dimensional DNA nanostructures may comprise nanodiscs or nanoplates.

The term “nanopore” as used herein refers to a pore of nanometer size ranging from fractions of a nanometer to tens of nanometers. In certain circumstance, the term “nanostructure” is used interchangeably with “nanopore”.

The term “folded protein” as used herein refers to a protein that has acquired some three-dimensional shape after translation of the polypeptide chain from which it is formed (the primary structure), by which the protein becomes biologically functional. The term may refer to the secondary structure of the protein which is typically the first stage of the folding process where local three-dimensional structures are formed, for example, alpha helices or beta sheets. The term may more typically refer to the tertiary structure of a protein where the secondary structures of the protein have folded to stabilize the structure through hydrophobic or covalent interactions. The term also encompasses proteins having a quaternary structure where one or more protein subunits are assembled. As appropriate, the folded protein may also be termed the native protein structure, and may be the form of the protein that exhibits its biological function. The folded protein may unfold in some contexts such as in heating.

The term “toehold mediated strand displacement” or “TMSD” refers to an enzyme-free molecular tool to exchange one strand of DNA or RNA (output) with another strand (input). It is based on the hybridization of two complementary strands of DNA or RNA via Watson-Crick base pairing (A-T/U and C-G) and makes use of a process called branch migration. TMSD starts with a double-stranded DNA complex composed of the original strand and the protector strand. The original strand has an overhanging region, the so-called “toehold”, which is complementary to a third strand of DNA referred to as the “invading strand”. The invading strand is a sequence of single-stranded DNA (ssDNA) which is complementary to the original strand. The toehold regions initiate the process of TMSD by allowing the complementary invading strand to hybridize with the original strand, creating a DNA complex composed of three strands of DNA. After the binding of the invading strand and the original strand occurred, branch migration of the invading domain then allows the displacement of the initial hybridized strand (protector strand). The protector strand can possess its own unique toehold and can, therefore, turn into an invading strand itself, starting a strand-displacement cascade.

The term “membrane” refers to an enclosing or separating selectively-permeable boundary, partition, barrier or film. The membrane has two sides or surfaces which may be named the cis and trans side respectively. The membrane is thin, allowing it to be spanned by the nanopore. The arrangement of the membrane is not limited and may in any form, for example, a liposome, vesicle or as a planar or a non-planar sheet. Lipid bilayer is an example of membranes useful in the present application.

### Nanopore

Molecular traffic across lipid membranes is an important process in cell biology that involves specialized biological pores with a great variety of pore diameters. The biological function of shuttling molecular cargo may be exploited by engineering pores. The pores may be further used for next-generation portable DNA sequencing and biosensing. Individual molecules that passing through a membrane-spanning pore may cause detectable changes in the ionic pore current. Narrow pores are suitable for sequencing as their 1-2 nm wide channels match the dimension of individual elongated translocating DNA strands. Wider pores are needed for transporting some cargo types, such as longer pieces of DNA or RNA, peptides, proteins, antibodies, and nanoparticles, which are difficult to get into cells. For example, 5-10 nm wide pores may allow transport of point-of-care diagnostics, and can be used to release therapeutic proteins from drug-delivery vesicles.

Biological pores are not suitable for these applications, because they may be not sufficiently wide for protein transport, or structurally complex, while de novo protein design of pores is currently too challenging. Synthetic nanopores solely composed of DNA are an attractive alternative towards a wider lumen given the ease of rationally designing defined nanoscale architectures with DNA nanotechnology.

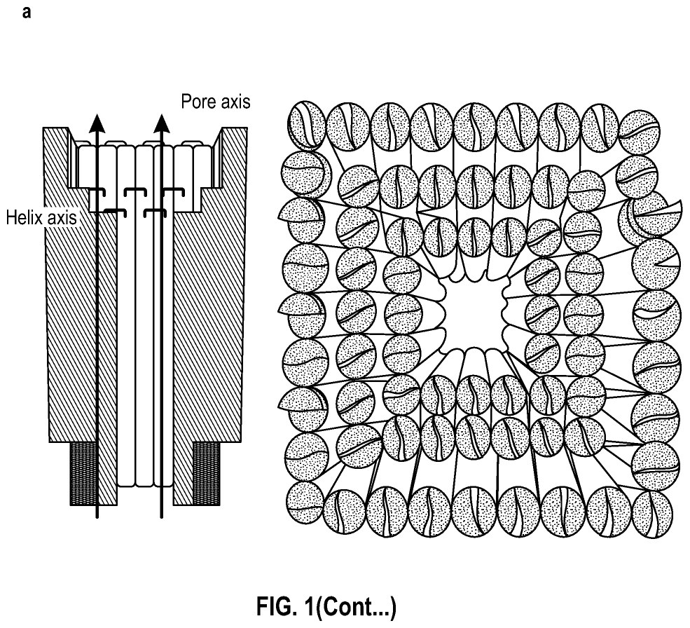

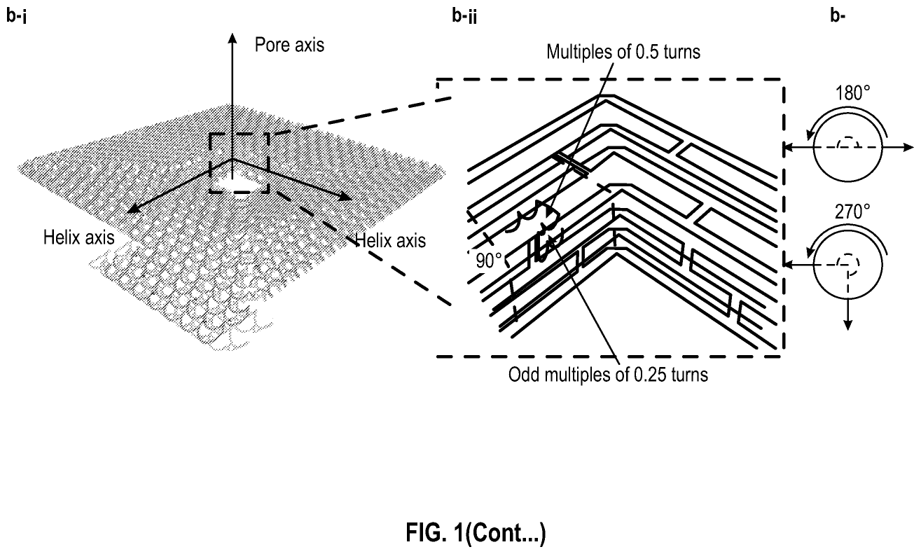

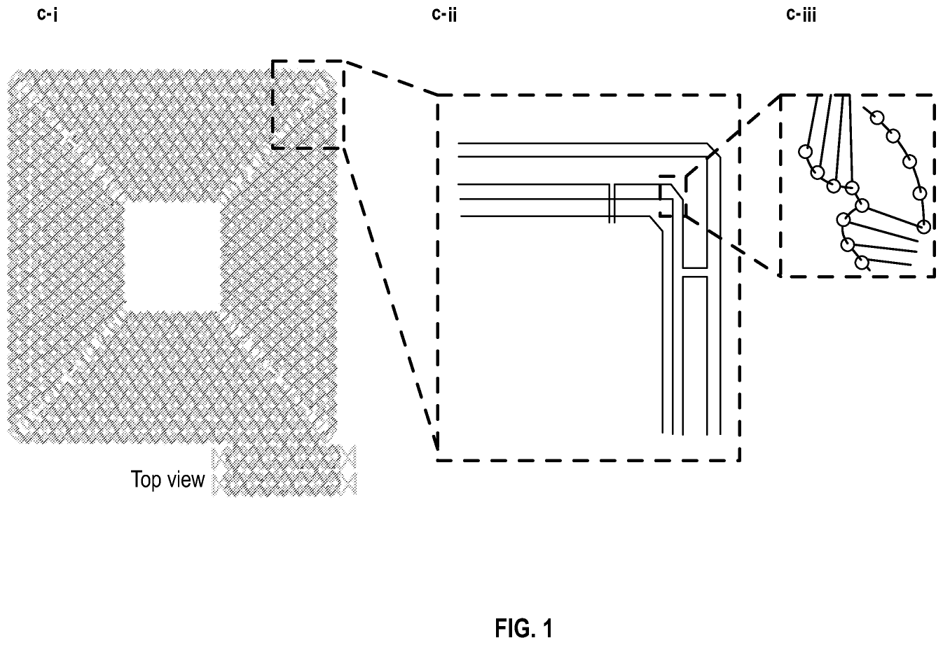

Reference de novo designs of DNA nanopores resulted barrel-like and constitutively open protein pores of a few nm width, which limits their utility in delivering agents into a cell. For example, Diederichs, T. et al. designed a DNA nanopore with a 7.5 nm wide central channel, whose duplex helices are routed ‘vertically’, i.e., at 90 degree, relative to the membrane plane (FIG. 1A)⁴⁹.

More recently, Fragasso A et al., produced a rigid octagonal ring-like with ‘horizonal’ routing, consisted of four single-duplex layer DNA origami plates (Fragasso A, De Franceschi N, Stommer P, van der Sluis E O, Dietz H, Dekker C. Reconstitution of Ultrawide DNA Origami Pores in Liposomes for Transmembrane Transport of Macromolecules [published online ahead of print, 2021 Jun. 25]. ACS Nano. 15(8), 12768-12779, (2021)). Such a design enables the fabrication of large pores having an inner diameter as large as 30 nm. However, the authors found it difficult to insert these wide pores into lipid layers, although 32 cholesterol modifications were made on the outer side the pore. The nanopores were finally inserted into the lipid membrane of giant unilamellar vesicles (GUVs) by administering the pores concomitantly with vesicle formation in an inverted-emulsion cDICE technique.

To overcome these problems, and to provide a wide pore that can readily insert into the lipid bilayers, the present inventors have designed and produced a nanostructure in horizontal routing with a large central pore or channel and a large bottom surface area available for cholesterol modification.

An exemplary square nanopore of the disclosure has a size of about 70 nm×70 nm, and contains a square shaped central pore having a size of about 20 nm×20 nm. The DNA nanopore of the disclosure with a cross-section of 416 nm² which is significantly higher than biological and reference DNA nanopores. In particular, the central pore size of the DNA nanostructures of this disclosure allows for transport of small molecules such as Atto 633, and folded proteins such as green fluorescent proteins (GFP). As described in the Examples herein, although atto-633 and GFP differs in hydrodynamic diameter, both show similar rate of influx %, perhaps because the nanopore of the disclosure is large enough compared to both Atto 633 and GFP.

The nanopore of the disclosure may comprise one or more hydrophobic moieties that act as anchors to attach or connect or anchor the hydrophilic DNA nanopore to the generally hydrophobic membrane (lipid bilayer, polymer or solid state). The hydrophobic anchors are attached to the nanopore, i.e., the nanopore may be hydrophobically modified. Suitably attachment is via polynucleotides, suitably DNA polynucleotide strands that carry the hydrophobic moiety, suitably a lipid such as cholesterol, at the 5′ or 3′ terminus. The term “hydrophobic” refers to a molecule having a polar character including organic molecules and polymers. Examples are saturated or unsaturated hydrocarbons. The molecule may have amphipathic properties. Hydrophobical modification refers to the modification (joining, bonding or otherwise linking) of a polynucleotide strand with one or more hydrophobic moieties. A “hydrophobic” is a hydrophobic organic molecule. The hydrophobic moiety may be any moiety comprising non-polar or low polarity aliphatic, aliphatic-aromatic or aromatic chains. Suitably, the hydrophobic moieties utilized in the present application encompass molecules such as long chain carbocyclic molecules, polymers, block co-polymers, and lipids. The term “lipid” refers to fatty acids and their derivatives (including tri-, di-, monoglycerides, and phospholipids), and sterol-containing metabolites such as cholesterol. The hydrophobic moieties comprised within the embodiments of the present invention are capable of forming non-covalent attractive interactions with phospholipid bilayers, such as the lipid-based membranes of cells and act as membrane anchors for the nanopore. According to certain embodiments of the present invention suitable hydrophobic moieties, such as lipid molecules, possessing membrane anchoring properties may include sterols (including cholesterol, derivatives of cholesterol, phytosterol, ergosterol and bile acid), alkylated phenols (including methylated phenols and tocopherols), flavones (including flavanone containing compounds such as 6-hydroxyflavone), saturated and unsaturated fatty acids (including derivatives such as lauric, oleic, linoleic and palmitic acids), and synthetic lipid molecules (including dodecyl-beta-D-glucoside). The anchors for the polymer membrane may be the same as for lipid bilayers or they may be different. The specific hydrophobic moiety anchor may be selected based on the binding performance of the membrane chosen.

These hydrophobically-modified anchor strands hybridize via “adaptor” polynucleotide strands to corresponding sections of the polynucleotide sequence forming the scaffold section of the nanopore. Alternatively, the hydrophobic anchors are assembled with the nanopore using hydrophobically-modified polynucleotides. The number of hydrophobically-modified anchors on a single nanopore is not limited. Cholesterol has been found to be a particularly suitable hydrophobic moiety for use as an anchor in the present disclosure. The use of other lipids as anchors is contemplated, although it may be expected that there is a particular preference for a particular hydrophobic moiety, and a given number of hydrophobic anchors, for a given membrane.

In certain embodiments, the nanopore of the disclosure is decorated with cholesterol-carrying oligonucleotides.

In addition, the nanopore of the disclosure may be further modified with detectable labels, for e.g., nanopore tracing and lid status check. In certain embodiments, the nanopore is decorated with fluorophone-carrying oligonucleotides. Modification may be made to the scaffold and/or staple strands prior to assembly of the nanopore.

The membrane in which the nanopore of the disclosure may be inserted into may be of any suitable type. Depending on the intended use, the membrane may a lipid bilayer or a polymer sheet or film or a solid state substrate. In solid state membranes, the substrate may already comprise apertures in which the nanopore sits thereby adapting the interior dimensions and channel width of the aperture. The membrane is typically hydrophobic to promote anchoring by the hydrophobic anchors.

Lipid bilayers are ubiquitous in biological organisms. It is envisaged that nanopores according to the disclosure may be inserted into a lipid membrane of a target cell or vesicle to facilitate translocation of specific folded proteins across the membrane. The specificity of the translocation to certain folded proteins may be controlled through variation of the size of the channel in the nanopore.

Proprietary and non-proprietary synthetic polymer films or sheets are widely used in nanopore sequencing methods such as the MinION system sold by Oxford Nanopore Technologies. The ability of nanopores to insert into polymer membranes of this type would allow these systems to be adapted for folded protein sensing applications. The polymer membrane may be formed of any suitable material. Typically, synthetic membranes are composed of amphiphilic synthetic block copolymers. Examples of hydrophilic block copolymers are poly(ethylene glycol) (PEG/PEO) or poly(2-methyloxazoline), while examples of hydrophobic blocks are polydimethylsiloxane (PDMS), poly(caprolactone) (PCL), poly(lactide) (PLA), or poly(methyl methacrylate) (PMMA). In embodiments, the polymer membrane used may be formed from the amphiphilic block copolymer poly 2-(methacryloyloxy)ethyl phosphorylcholine-b-disisopropylamino) ethyl methacrylate (PMPC-b-PDPA). DNA nanopores may be inserted into the walls of such polymersomes through incubation.

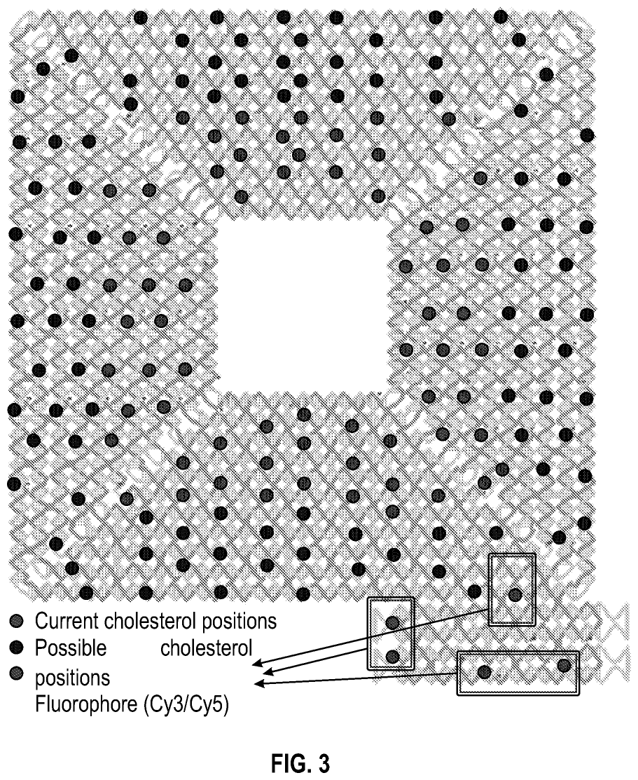

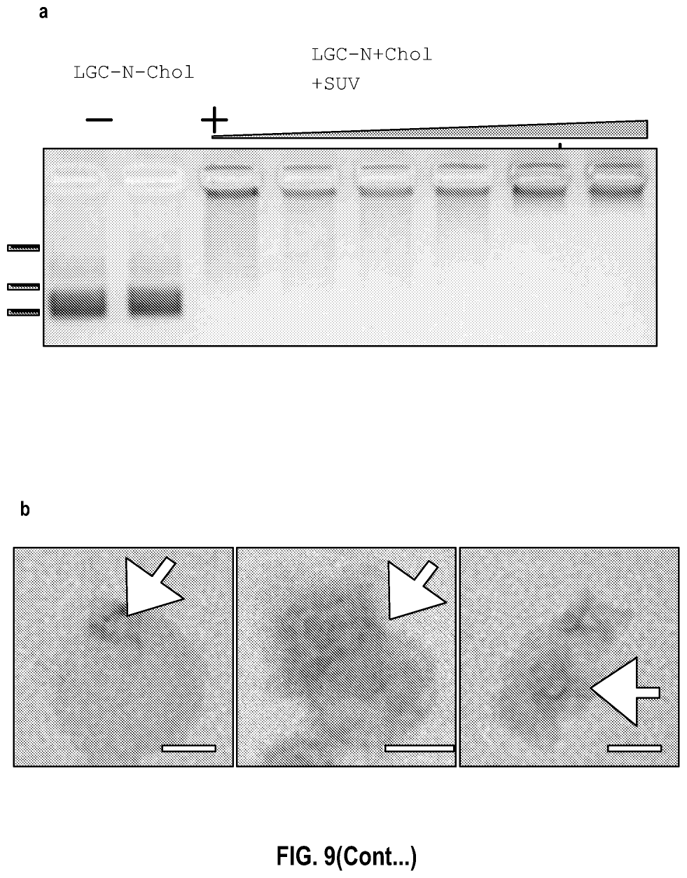

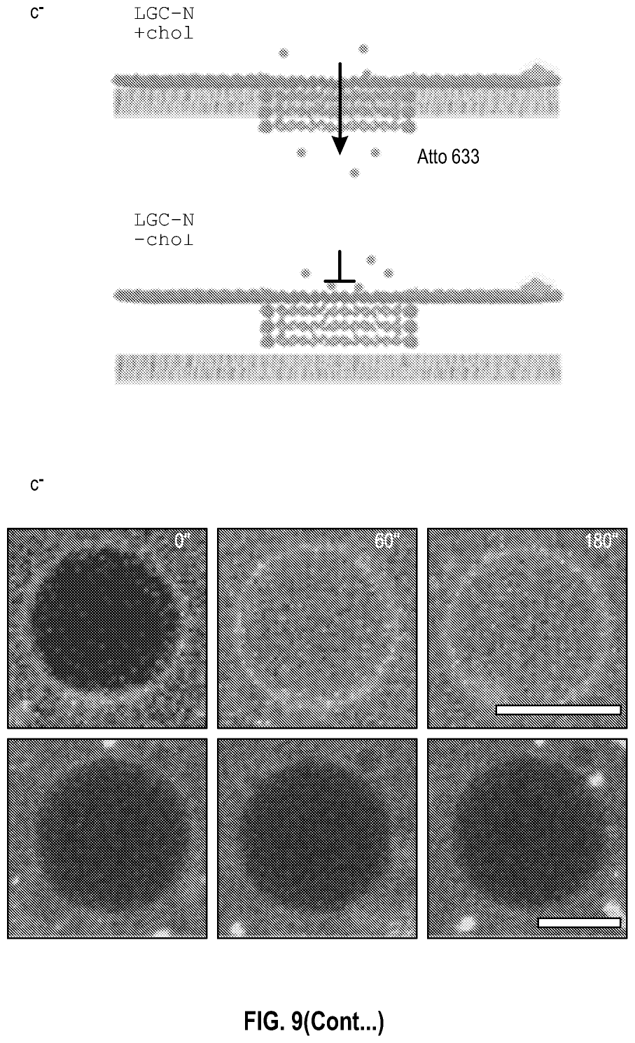

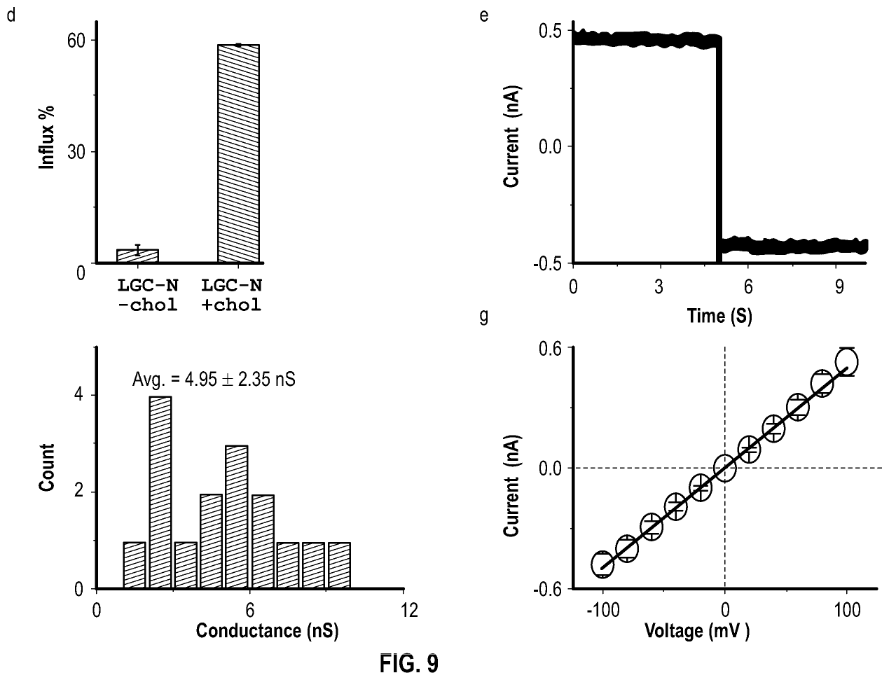

As shown in FIG. 3, an exemplary nanopore of this disclosure was designed with 64 cholesterol positions on the bottom surface of the base plate. When these positions were modified with cholesterols, the exemplary nanopore bound efficiently to lipid membranes of slowly migrating vesicles, and the central pore/channel successfully inserted into and punctured membranes as indicated by Atto633 signals that increased within GUVs over the full incubation time (FIG. 9c-ii, top). Further, also as shown in FIG. 3, there are still space for more cholesterol modifications, which may facilitate insertion of even wider pores into the lipid bilayers.

Without wishing to be bound to theories, the nanopore of the disclosure readily inserts into the lipid bilayers probably due to the large area with cholesterol modification enough to draw it into the bilayers. The nanopore's simple and thin structure may also attribute to this favorable feature.

The nanopores of the disclosures may be tunable to permit adaption to different pore channel sizes. The pores may be composed of any suitable nucleic acids. Nanopores formed from one or more DNA duplexes may be suitable as they are an excellent construction material for rationally designing nanoscale architectures of defined size (Langecker M., et al. Science 338, 932-936 (2012); Burns J., Stulz E., Howorka S. Nano Lett. 13, 2351-2356 (2013); Burns J. R., Al-Juffali N., Janes S. M., Howorka S. Angew. Chem. Int. Ed. 53, 12466-12470 (2014); Burns J. R., Seifert A., Fertig N., Howorka S. Nat. Nanotechnol. 11, 152-156 (2016)).

In additional to the exemplary ones, the nanopore of the disclosure may be designed to have any suitable shape, although generally it is horizontally routed and contains cholesterol modification positions at the bottom surface of the base plate sheet. In an embodiment, the nanopore of the disclosure may comprise a) a base plate sheet comprising a plurality of single-duplex layer DNA origami plates, and a pore configured within the center of the base plate sheet and which traverses each of the single-duplex layer DNA origami plates. A lid may be configured to be on the top surface of the base plate sheet.

The base plate sheet in the nanopore of the disclosure may have dimensions of any suitable size. Typically, the base plate sheet has a height that approximately matches the thickness of the membrane in which it resides. The thickness of biological lipid bilayer membranes can range from around 3.5 to 10 nm. The thickness of membrane composed of amphiphilic synthetic block copolymers shows a wider range from 5 to 50 nm (C. LoPresti, H. Lomas, M. Massignani, T. Smart, G. Battaglia, J. Mater. Chem. 2009, 19, 3576-3590). Therefore suitably, the base plate sheet may have a height of at least around 3.5 nm, although it may be possible to have a membrane-spanning region with a height as low as 3 nm, 2.5 nm, 2 nm 1.5 nm or 1.0 nm or less. Suitably, the membrane-spanning region may have a height of at least 5 nm. The membrane-spanning region may have a height of at most 100 nm, 90 nm, 80 nm, 70 nm, 60 nm, 50 nm, 40 nm, 30 nm, 20 nm or 10 nm or less. Suitably the membrane-spanning region has a maximum height of 50 nm for synthetic polymer layers, and a maximum height of 10 nm for lipid bilayers. In certain embodiments, the base plate sheet of the disclosure is composed of a single-duplex layer, which is thinner than the lipid bilayer, despite of a four single-duplex layer-thick wall extending from the base plate sheet.

The central pore, channel or lumen that passes through the extending wall has a cross-sectional profile parallel to the membrane and perpendicular to the pore axis. This cross-sectional profile may be of any shape and dimensions. The shape and dimensions of the pore may be consistent for its entire length or may vary. Suitably, the pore has a consistent cross-sectional profile and size for its entire length. Suitably, the cross-sectional profile of the pore is a quadrilateral, typically a square, or at least generally circular. The minimum opening or width of the pore in this cross-section is suitable to allow access for a folded protein. Typically, the minimum opening or width of the pore is at least 5 nm, 6 nm, 6.5 nm, 7 nm, 7.5 nm, 8 nm, 8.5 nm, 9 nm, 9.5 nm, 10 nm, 11 nm, 12 nm, 13 nm, 14 nm, 15 nm, 16 nm, 17 nm, 18 nm, 19 nm, 20 nm, 25 nm or more. Suitably the opening is between around 5 nm and around 20 nm. Typically the opening is around 5 nm and around 10 nm. The maximum opening of the channel is limited only by the need to maintain structural integrity of the pore and to obtain an electrical read-out when a molecule of interest passes through. Suitably, the cross-sectional area of the minimum opening of the pore is at least 20 nm², 25 nm², 30 nm², 35 nm², 40 nm², 45 nm², 50 nm², 60 nm², 70 nm², 80 nm², 90 nm² or 100² or more.

To achieve optimum performance, the variation in the channel size should be minimized from pore to pore. Even small variation in surface area can lead to a significant discrepancy in ion flow through a given pore, both in the open state (devoid of any target analyte), or in the bound state (with target analyte present in, or proximate to, the pore). Such variation in ion flow leads to a lower signal to noise ratio in the electrical read-out thereby reducing the sensitivity of detection.

The nanopore of the disclosure may be further provided with a lid that allows precisely timed, stimulus-controlled transport of folded and functional proteins across bilayer membranes, by e.g., a key and reverse key mechanism. The lid may comprise a single-duplex layer and is linked to the base plate sheet by flexible hinges each comprising a 4-nucleotide single stranded region. Several designs with rigid hinge did not lead to successful closure of the lid, perhaps because of strain in the hinge region (data not shown).

In certain embodiments, the lid and the base plate comprise a first-half lock and a second-half lock, respectively, which two comprise single stranded DNA sequences complementary to each other. One or more, e.g., two, locks may be formed by the hybridization of two sets of complementary strands to keeps the lid closed. Each lock contains a strand with a toehold. An opening key opens the lock(s) by toehold mediated strand displacement (TMSD), leading to the open lid. The opening key also contains a toehold which then initiates a second TMSD when a closing key is added. As a result, the opening key dissociates from the lock strands and lock strands reseal to form the closed lid.

The data in the Examples has shown the no lid and dynamically opened lid LGC pores had similar rate of influx % whereas the closed lid pore showing no influx % indicated that the open lid was equivalent to no lid in terms of passage of molecules through the LGC channel.

The reconfigurable nanomechanical lid enables control over transport of various cargoes ranging from small molecule dye to folded proteins of up to 5.8 nm hydrodynamic diameter. This exceeds the natural analogue-ligand gated ion channels which have much narrower pore dimension. The lidded nanopore of the disclosure can reversibly capture proteins and study their interaction with free-flowing ligands (Soskine, M., Biesemans, A. & Maglia, G. Single-molecule analyte recognition with clya nanopores equipped with internal protein adaptors. J. Am. Chem. Soc. 137, 5793-5797, (2015); Van Meervelt, V. et al. Real-time conformational changes and controlled orientation of native proteins inside a protein nanoreactor. J. Am. Chem. Soc. 139, 18640-18646, (2017)) or real-time protein folding and unfolding. The reversibly controlled flow of cargo by a nanomechanical lid may also be exploited to transport bioactive cargo into cells, or to construct cell-cell communication for artificial gap junctions and to enable integration of artificial tissues with living cells and tissues for tissue engineering.

### Nanopore Construction

Suitably, the nanopore of the present disclosure is assembled via the ‘scaffold-and-staple’ approach. In this important route to nucleic acid nanostructure, in particular, DNA nanostructures, DNA is utilized as a building material in order to make nanoscale three dimensional shapes. Assembly of these complex nanostructures from a plurality of un-hybridized linear molecules is typically referred to as ‘DNA origami’, although the technique is equally applicable to other nucleic acids.

The basic principle of DNA origami design is to translate the desired final shape into the folding route of a given scaffold and generate corresponding staple sequences that can fulfil the folding (Dey, S., Fan, C., Gothelf, K. V. et al. DNA origami. Nat Rev Methods Primers 1, 13 (2021)).

Different software have been developed for designing DNA origami structures. The first-generation DNA origami design tools, such as caDNAno8, were developed for designing various 2D and 3D origami structures. The caDNAno program is a software for designing DNA origami. Other software include Tiamat, SARSE-DNA, Nanoengineer-1, Hex-tiles, GIDEON, and K-router. The first-generation software require manual scaffold routing, and manual- or semi-automated scaffold and staple crossover creation, requiring extensive expertise on this structure type and more technical knowledge for design of DNA origami. Second-generation design software have been developed to be more user-friendly and demand less technical knowledge than their first-generation counterparts. The second-generation software is able to generate staple sequences in an automated fashion from user-provided 3D designs. vHelix is another software in this category and also contains an integrated simulation platform that can predict the folding of the designed structures in standard DNA origami folding buffers. Other software such as DAEDALUS47 and TALOS9 for 3D origami and vHelix-BSCOR48, PERDIX49 and METIS10 for 2D origami are also available. Two new software have been reported that combine features from the first and second-generation software. ATHENA integrates features of other existing second-generation software, specifically that of DAEDALUS, PERDIX, TALOS and METIS. Adenita is an open source platform that combines almost all of the first and second-generation design software capabilities. It can design lattice-based wireframes, multilayered structures, free-form tiles and single-stranded tiles. Adenita also contains an integrated simulation platform to predict the stability of the designed structures in buffer after their formation. Being integrated with the commercial nanoscale simulation software SAMSON, Adenita is the only software that also accommodates other biomolecules such as protein, lipid or drug molecules.

Following nanostructure design, it is important to predict the folding of designed origami computationally. All-atom molecular dynamics simulation has been successfully used for characterizing the structural, mechanical and ionic conductive properties of DNA origami in microscopic detail at the DNA single base pair level. A comprehensive web server-based package, oxDNA.org is as an entirely web-based application that uses rigid-body simulation to predict more advanced structural features such as the root mean square fluctuation structure, average hydrogen-bond occupancy, distance between user-specified nucleotides and angle between each duplex in the nanostructure. oxView, the graphical user interface of oxDNA, also offers de novo design of DNA nanostructures that is particularly useful when manipulating previously published designs for specific applications based on oxDNA simulations.

The most commonly used scaffold is the m13mp18 viral genome 7,249 nucleotides isolated from the M13 phage. Other typical scaffolds include p7308, p7560 and p8064, all derived from M13, which provide alternative scaffold lengths and sequences. These scaffolds are commercially available from e.g., Guild Biosciences, Integrated DNA Technologies, and New England Biolabs, and can also be custom-made using asymmetrical PCR, using enzymatic single-strand digestion of PCR amplified double-stranded DNAs or by purifying phage-derived single-stranded genomic DNAs. Breaking away from the M13 genome in terms of production of scaffolds with custom size and sequence could provide more design possibilities. Clearly, the size of a single DNA origami structure is limited by the length of the scaffold. Efforts in scaling up the origami's dimention include the use of longder scaffold strands or shor scaffold-parity strands. The latter one uses a set of randomly generated sequences typically 42 nucleotides long that are complementary to segments of staples extending from the origami shape and partially hybridized to the scaffold. In this respect, additional helical layers may be bound to the scaffold-related origami structure.

DNA origami structures are folded via one-pot self-assembly. To reduce non-specific aggregates, the staple strands are provided at a concentration 10-20× higher than that of the scaffold strands. For dynamic DNA stracures such as the lidded nanopore of the disclosure, the staples involved in dynamic reconfiguration are often purified by denaturing PAGE to ensure their incorporation at the desired locations. The staple to scaffold ratio is usually 1.5-2, to promote intramolecular over intermolecular interactions. The mixtures undergoes a thermal annealing process where it is heated to near boiling for a short time and then cooled to allow spontaneous self-assembly. The annealing procedure may last for a few hours for a 2D structure, and maybe several days for a multilayer 3D origami. Stepwise assembly may be used for generation of structures integrated with other functional materials, or hierarchical structures.

The exemplary nanopore of the disclosure as used in the Examples may be prepared by using the core strands, strands for cholesterol modification, strands for fluorophore modification, and/or lock strands (for lidded nanopore). The nanopore may be decorated with the cholesterol modified strands, and/or fluorophore modified strands.

Agarose gel electrophoresis may be used to assess whether the self-assembly succeeded and to what extent. Differences can be found in the gel migration rates between correct products and by-products.

The formed nanostructures may be purified and enriched before used for any applications, using gel purification, ultrafiltration, polyethylene glycol (PEG) precipitation, ultracentrifugation, or size-exclusion chromatography.

The formed nanostructures should also be characterized for their structures and functions, by force and optical-based methods, such as atomic force microscopy (AFM), transmission electron microscopy (TEM), cryo-EM, single-molecule fluorescence microscopy and, more recently, single-molecule force measurements. AFM is of highfidelity, and has a lateral resolution up to ca. 1-2 nm, and imaging can be done in fluid or in air. Modified AFM tips can be used to study the mechanical and elastic properties of the DNA nanostructure, and high-speed AFM can probe structural dynamics in real time. DNA samples can be fixed on the mica surface using nickel acetate, and the Ni²⁺ concentration should be adjusted for origami type, i.e., higher Ni²⁺ for smaller structures. However, AFM is not suitable for 3D or multiplayer structures, and is time-consuming. TEM is best for 3D structures with highest resolution, but may cause sample deformation.

### Use of Nanopores

The ability to create nanopores with controlled pore sizes and insert them into natural or synthetic membranes allows the construction of customised selectively-permeable membranes where control over the lumen dimensions, and optionally, other features of the pores enables control over which biomolecules are able to pass through these membranes. Essentially, the nanopores of this disclosure act as molecular trocars, wherein the DNA nanopore affixes to the exterior surface of a lipid bilayer (e.g., cell wall), and allows for selectively gated transport of an agent into the cell. Of particular utility in the present application is the increased size of the nanopores that allow more cholesterol modification and the large size of the central pore that allows large biomolecules, including large globular proteins, to pass through. This has potential utility in medicine alongside other uses in the field of biology. For example, there are several large, i.e., 10-30 nm diameter membrane pores that are either produced by immune cells to kill bacteria, such as in the ‘complement system’, a part of the innate immune system that enhances (complements) the ability of antibodies and phagocytic cells to clear microbes and damaged cells from an organism, promotes inflammation, and attacks the pathogen's plasma membrane. In particular to the C9 protein forms a large pore that together with other accessory proteins forms the membrane attack complex. Membrane poration is also used by pathogenic bacteria to attack and kill eukaryotic host cells. Examples of these bacterial pores are Cholesterol-dependent Cytolysin which includes perfrinoglysin or listeriolysin O.

As an example, vesicles formed of natural or synthetic polymers comprising nanopores of the disclosure may be used as nanoreactors. It is envisaged that substrate proteins or biomolecules may enter the vesicle interior through the nanopore where encapsulated enzymes in the interior of the vesicle enable a desired reaction to take place within the vesicle. As an example, encapsulated protease enzymes, such as trypsin may be retained inside a vesicle where the relatively large and controllable size of the nanopores could allow some substrate proteins but not others to enter for degradation, thereby protecting particular proteins from indiscriminate digestion. Similarly, control over the release of cargo contained within similar vesicles, such as protein or peptide based pharmaceutical agents would be possible with membranes with nanopores which allow passage of the cargo.

When functioning as a molecular sensor, the vesicles with the nanopores of the disclosure may detect and characterize an analyte, espectically larger analytes such as unfoled proteins, given the large pore dimensions of the nanopores disclosed herein. The nanopores of the disclosure may be modified to reduce the pore diameter making the nanopore suitable for the detection of analytes having a smaller size, although a large pore may be also applicable to detection of small molecules. Also, the nanopore of the disclosure may be with a larger pore to allow passage of larger molecules (e.g., an agent). The analyte may be caused to fully or partially translocate the pore. The analyte may for example be held or lodged within the pore or pore entrance. Measurement of a signal, for example the change in ion flow during translocation may be used to detect or characterize the analyte. Alternatively the analyte may be caused to pass across the entrance of the nanopore in order to detect or characterize it.

Examples of analytes that may be detected or characterized are folded or unfolded proteins, DNA-protein constructs such as nucleosomes and polynucleotides such as deoxyribonucleic acid (DNA) or ribonucleic acid (RNA), polysaccharides and synthetic polymers. The analytes may be modified with a detectable label. The label may be optically detectable such as a fluorophore.

The presence or absence of the analyte may be detected. Alternatively the analyte may be characterized, for example in the case of a nucleic acid, the sequence of the nucleotides may be determined from characteristic disruptions in the measured signal over time. In the case of proteins, aspects of the protein structure may be determined. The protein may be unfolded prior to detection using the nanopore. An example of such is disclosed in PCT/US2013/026414, herein incorporated by reference.

The nanopore of the disclosure may also be used for targeted delivery of e.g., therapeutic agents. This can be done by filling a vesicle with the nanopore of the disclosure with a therapeutic agent, through i) contacting a therapeutic agent and a lidded nanostructure of the disclosure having a pore in a closed configuration with the vesicle, wherein the lipid membrane anchor forms a complex with the lipid bilayer of the vesicle, ii) presenting a key single-stranded DNA sequence to the nanostructure, wherein the lid opens and the pore is in an open configuration, iii) allowing the agent to traverse through the pore through the lipid bilayer, and vi) presenting a reverse key single-stranded DNA sequence to the nanostructure, wherein the lid closes. Prior to administration of the vesicles, an opening key is modified to bind a target protein such as a tumor associated antigen by e.g., linking to a peptide or oligonucleotides specific for the target protein, and the modified opening key is administered. The opening key may be further modified to avoid quick degradation, in vitro and/or in vivo. The vesicle may be a small unilamellar vesicle or a giate unilamellar vesicle

### Therapeutic Agents

In some embodiments, the therapeutic agent is selected from a small molecule, a peptide, a small protein, or a small nucleic acid. In some embodiments, the small molecule is selected from a steroid (e.g., cholesterol), a hormone, a mono- or polysaccharide, glucose, amino acids, lipids, glycosides, alkaloids, and natural phenols.

In some embodiments, the therapeutic agent is an anticancer agent. The anticancer is agent is selected from: Abraxane (chemical name: albumin-bound or nab-paclitaxel), Adriamycin (chemical name: doxorubicin), carboplatin (brand name: Paraplatin), Cytoxan (chemical name: cyclophosphamide), daunorubicin (brand names: Cerubidine, DaunoXome), Doxil (chemical name: doxorubicin), Ellence (chemical name: epirubicin), fluorouracil (also called 5-fluorouracil or 5-FU; brand name: Adrucil), Gemzar (chemical name: gemcitabine), Halaven (chemical name: eribulin), Ixempra (chemical name: ixabepilone), methotrexate (brand names: Amethopterin, Mexate, Folex), Mitomycin (chemical name: mutamycin), mitoxantrone (brand name: Novantrone), Navelbine (chemical name: vinorelbine), Taxol (chemical name: paclitaxel), Taxotere (chemical name: docetaxel), thiotepa (brand name: Thioplex), vincristine (brand names: Oncovin, Vincasar PES, Vincrex), and Xeloda (chemical name: capecitabine). In certain embodiments, the chemotherapeutic agent is selected from: Abraxane (Paclitaxel (with albumin) Injection), Adriamycin (Doxorubicin), Afinitor (Everolimus), Alecensa (Alectinib), Alimta (PEMETREXED), Aliqopa (Copanlisib), Alkeran Injection (Melphalan), Alunbrig (Brigatinib), Aredia (Pamidronate), Arimidex (Anastrozole), Aromasin (Exemestane), Arranon (Nelarabine), Arzerra (Ofatumumab), Avastin (Bevacizumab), Bavencio (Avelumab), Beleodaq (Belinostat), Besponsa (Inotuzumab Ozogamicin), Bexxar (Tositumomab), BiCNU (Carmustine), Blenoxane (Bleomycin), Blincyto (Blinatumomab), Bosulif (Bosutinib), Braftovi (Encorafenib), Busulfex (Busulfan), Cabometyx (Cabozantinib), Calquence (Acalabrutinib), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Cinqair (Reslizumab), Clolar (Clofarabine), Cometriq (Cabozantinib), Copiktra (Duvelisib), Cosmegen (Dactinomycin), Cotellic (Cobimetinib), Cyramza (Ramucirumab), CytosarU (Cytarabine), Cytoxan (Cytoxan), Cyclophosphamide, Dacogen (Decitabine), Darzalex (Daratumumab), DaunoXome (Daunorubicin Lipid Complex), Daurismo (Glasdegib), Decadron (Dexamethasone), DepoCyt (Cytarabine Lipid Complex), Dexamethasone Intensol (Dexamethasone), Dexpak Taperpak (Dexamethasone), Docefrez (Docetaxel), Doxil (Doxorubicin Lipid Complex), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Emend (Fosaprepitant), Empliciti (Elotzumab), Erbitux (Cetuximab), Erivedge (Vismodegib), Erleada (Apalutamide), Erwinaze (Asparaginase Erwinia chrysanthemi), Ethyol (Amifostine), Etopophos (Etoposide), Eulexin (Flutamide), Fareston (Toremifene), Farydak (Panobinostat), Faslodex (Fulvestrant), Femara (Letrozole), Firmagon (Degarelix), FloPred (Prednisolone), Fludara (Fludarabine), Folex (Methotrexate), Folotyn (Pralatrexate), FUDR (FUDR (floxuridine)), Gazyva (Obinutuzumab), Gemzar (Gemcitabine), Gilotrif (Afatinib), Gleevec (Imatinib Mesylate), Halaven (Eribulin), Herceptin (Trastuzumab), Hexalen (Altretamine), Hycamtin (Topotecan), Hycamtin (Topotecan), Hydrea (Hydroxyurea), Ibrance (Palbociclib), Iclusig (Ponatinib), Idamycin PFS (Idarubicin), Idhifa (Enasidenib), Ifex (Ifosfamide), Imbruvica (Ibrutinib), Imfnzi (Durvalumab), Imlygic (Talimogene Laherparepvec), Inlyta (Axitinib), Intron A alfab (Interferon alfa-2a), Iressa (Gefitinib), Istodax (Romidepsin), Ixempra (Ixabepilone), Jakafi (Ruxolitinib), Jevtana (Cabazitaxel), Kadcyla (Ado-trastuzumab Emtansine), Keytruda (Pembrolizumab), Kisqali (Ribociclib), Kyprolis (Carfilzomib), Lanvima (Lenvatinib), Leukeran (Chlorambucil), Leukine (Sargramostim), Leustatin (Cladribine), Lorbrena (Lorlatinib), Lupron (Leuprolide), Lynparza (Olaparib), Lysodren (Mitotane), Matulane (Procarbazine), Megace (Megestrol), Mekinist (Trametinib), Mektovi (Binimetinib), Mesnex (Mesna), Mustargen (Mechlorethamine), Mutamycin (Mitomycin), Myleran (Busulfan), Mylotarg (Gemtuzumab Ozogamicin), Navelbine (Vinorelbine), Nerlynx (Neratinib), Neulasta (filgrastim), Neulasta (pegfilgrastim), Neupogen (filgrastim), Nexavar (Sorafenib), Nilandron (Nilandron (nilutamide)), Ninlaro (Ixazomib), Nipent (Pentostatin), Nolvadex (Tamoxifen), Odomzo (Sonidegib), Oncaspar (Pegaspargase), Oncovin (Vincristine), Opdivo (Nivolumab), Panretin (Alitretinoin), Paraplatin (Carboplatin), Perjeta (Pertuzumab), Platinol (Cisplatin), PlatinolAQ (Cisplatin), Pomalyst (Pomalidomide), Portrazza (Necitumumab), Proleukin (Aldesleukin), Purinethol (Mercaptopurine), Reclast (Zoledronic acid), Revlimid (Lenalidomide), Rituxan (Rituximab), RoferonA alfaa (Interferon alfa-2a), Rubex (Doxorubicin), Rubraca (Rucaparib), Rydapt (Midostaurin), Sandostatin (Octreotide), Soltamox (Tamoxifen), Sprycel (Dasatinib), Stivarga (Regorafenib), Sutent (Sunitinib), Sylvant (Siltuximab), Synribo (Omacetaxine), Tabloid (Thioguanine), Taflinar (Dabrafenib), Tagrisso (Osimertinib), Talzenna (Talazoparib), Tarceva (Erlotinib), Targretin Capsules (Bexarotene), Tasigna (Decarbazine), Taxol (Paclitaxel), Taxotere (Docetaxel), Tecentriq (Atezolizumab), Temodar (Temozolomide), Tepadina (Thiotepa), Thioplex (Thiotepa), Tibsovo (Ivosidenib), Toposar (Etoposide), Torisel (Temsirolimus), Treanda (Bendamustine hydrochloride), Trelstar (Triptorelin), Tykerb (lapatinib), Unituxin (Dinutuximab), Valstar (Valrubicin), Varubi (Rolapitant), Vectibix (Panitumumab), Velban (Vinblastine), Velcade (Bortezomib), Venclexta (Venetoclax), Vepesid (Etoposide), Vepesid (Etoposide Injection), Verzenio (Abemaciclib), Vesanoid (Tretinoin), Vidaza (Azacitidine), Vincasar PFS (Vincristine), Vincrex (Vincristine), Vistogard (Uridine Triacetate), VitrakviI (Larotrectinib), Vizimpro (Dacomitinib), Votrient (Pazopanib), Vumon (Teniposide), Wellcovorin IV (Leucovorin), Xalkori (Crizotinib), Xeloda (Capecitabine), Xospata (Gilteritinib), Xtandi (Enzalutamide), Yervoy (Ipilimumab), Yescarta (Axicabtagene), Yondelis (Trabectedin), Zaltrap (Ziv-aflibercept), Zanosar (Streptozocin), Zejula (Niraparib), Zelboraf (Vemurafenib), Zevalin (Ibritumomab Tiuxetan), Zoladex (Goserelin), Zolinza (Vorinostat), Zometa (Zoledronic acid) Zortress (Everolimus), Zydelig (Idelalisib), Zykadia (Ceritinib), and Zytiga (Abiraterone). In some embodiments, the anti-cancer agent is selected from: Alkylating Agents: Altretamine, Bendamustine, Busulfan, Carmustine, Chlorambucil, Cyclophosphamide, Dacarbazine, Ifosfamide, Lomustine, Mechlorethamine, Melphalan, Procarbazine, Streptozocin, Temozolomide, Thiotepa, Trabectedin; Platinum Coordination Complexes: Carboplatin, Cisplatin, Oxaliplatin; Bleomycin, Dactinomycin, Daunorubicin, Doxorubicin, Epirubicin, Idarubicin, Mitomycin, Mitoxantrone, Plicamycin, Valrubicin, Antimetabolites: Antifolates: Methotrexate, Pemetrexed, Pralatrexate, Trimetrexate; Purine Analogues: Azathioprine, Cladribine, Fludarabine, Mercaptopurine, Thioguanine; Pyrimidine Analogues: Azacitidine, Capecitabine, Cytarabine, Decitabine, Floxuridine, Fluorouracil, Gemcitabine, Trifluridine/Tipracil; Biologic Response Modifiers: Aldesleukin (IL-2), Denileukin Diftitox, Interferon Gamma; Histone Deacetylase Inhibitors: Belinostat, Panobinostat, Romidepsin, Vorinostat; Hormonal Agents: Antiandrogens: Abiraterone, Apalutamide, Bicalutamide, Cyproterone, Enzalutamide, Flutamide, Nilutamide; Antiestrogens (including Aromatase Inhibitors): Anastrozole, Exemestane, Fulvestrant, Letrozole, Raloxifene, Tamoxifen, Toremifene; Gonadotropin Releasing Hormone Analogues: Degarelix, Goserelin, Histrelin, Leuprolide, Triptorelin; Peptide Hormones: Lanreotide, Octreotide, Pasireotide; Monoclonal Antibodies: Alemtuzumab, Atezolizumab, Avelumab, Bevacizumab, Blinatumomab, Brentuximab, Cemiplimab, Cetuximab, Daratumumab, Dinutuximab, Durvalumab, Elotuzumab, Gemtuzumab, Inotuzumab Ozogamicin, Ipilimumab, Mogamulizumab, Moxetumomab Pasudotox, Necitumumab, Nivolumab, Ofatumumab, Olaratumab, Panitumumab, Pembrolizumab, Pertuzumab, Ramucirumab, Rituximab, Tositumomab, Trastuzumab, Protein Kinase Inhibitors, Abemaciclib, Acalabrutinib, Afatinib, Alectinib, Axitinib, Binimetinib, Bortezomib, Bosutinib, Brigatinib, Cabozantinib, Carfilzomib, Ceritinib, Cobimetinib, Copanlisib, Crizotinib, Dabrafenib, Dacomitinib, Dasatinib, Duvelisib, Enasidenib, Encorafenib, Erlotinib, Gefitinib, Gilteritinib, Glasdegib, Ibrutinib, Idelalisib, Imatinib, Ivosidenib, Ixazomib, Lapatinib, Larotrectinib, Lenvatinib, Lorlatinib, Midostaurin, Neratinib, Nilotinib, Niraparib, Olaparib, Osimertinib, Palbociclib, Pazopanib, Pexidartinib, Ponatinib, Regorafenib, Ribocicib, Rucaparib, Ruxolitinib, Selumetinib, Sonidegib, Sorafenib, Sunitinib, Talazoparib, Trametinib, Vandetanib, Vemurafenib, Vismodegib, Zanubrutinib; Taxanes: Cabazitaxel, Docetaxel, Paclitaxel; Topoisomerase Inhibitors: Etoposide, Irinotecan, Teniposide, Topotecan; Vinca Alkaloids: Vinblastine, Vincristine, Vinorelbine; or Asparaginase (Pegaspargase), Bexarotene, Eribulin, Everolimus, Hydroxyurea, Ixabepilone, Lenalidomide, Mitotane, Omacetaxine, Pomalidomide, Tagraxofusp, Telotristat, Temsirolimus, Thalidomide, or Venetoclax.

In some embodiments, the therapeutic agent is a peptide. In some embodiments, the peptide is selected from: Nesiritide, Ceruletide, Bentiromide, Exenatide, Gonadorelin, Enfuvirtide, Vancomycin, Icatibant, Secretin, Leuprolide, Glucagon recombinant, Bivalirudin, Sermorelin, Gramicidin D, Insulin recombinant, Capreomycin, Salmon Calcitonin, Vasopressin, Cosyntropin, Bacitracin, Octreotide, Abarelix, Vapreotide, Thymalfasin, Insulin recombinant, Mecasermin, Cetrorelix, Teriparatide, Corticotropin, Pramlintide, Valirudin, Buserelin, Corticotropin, Cosyntropin, Enfuvirtide, Eptifibatide, Exenatide, Glatiramer, Gramicidin D, Lepirudin, Leuprolide, Liraglutide, Lucinactant, Nesiritide, Oxytocin, Pramlintide, Salmon Calcitonin, Secretin, Sermorelin, Teduglutide, Thymalfasin, Lepirudin, Bivalirudin, Leuprolide, Sermorelin, Goserelin, Pegfilgrastim, Anakinra, Gramicidin D, Insulin human, Desmopressin, Glucagon, Cetrorelix, Somatotropin, Vasopressin, Daptomycin, Octreotide, Abarelix, Oxytocin, Enfuvirtide, Glutathione, Pentagastrin, Ceruletide, Enalapril, Bacitracin, Gonadorelin, Nafarelin, Lisinopril, Mecasermin, Tetracosactide, Insulin detemir, Insulin glulisine, Spirapril, Oritavancin, Afamelanotide, Lancovutide, Larazotide, Caplacizumab, Romidepsin, Human C1-esterase inhibitor, Liraglutide, Degarelix, Buserelin, Ganirelix, Histrelin, Lanreotide, Triptorelin, Ocriplasmin, Certolizumab pegol, Dulaglutide, Metreleptin, Peginterferon beta-1a, Protirelin, Enalaprilat, Insulin degludec, Deslorelin, Bremelanotide, Setmelanotide, Angiotensin II, Vosoritide, Barusiban, Semaglutide, Filgrastim, Lypressin, Glutathione disulfide, Voclosporin, Cyclosporine, Polymyxin B, Human interferon beta, Somapacitan, Antipain, Etanercept, Exenatide, Luspatercept, Dotatate gallium Ga-68, Ornithine, Polygeline, Indium In-Ill satumomab pendetide, Alpha-1-proteinase inhibitor, Chymostatin, Arylomycin A2, ACV tripeptide, Mdl 101,146, Cyclo(his-pro), N-[(6S)-6-Carboxy-6-(glycylamino)hexanoyl]-D-alanyl-D-alanine, GE-2270A, Argifin, Skf 107457, Cyclotheonamide A, Argadin, Aeruginosin 98-B, Cyclo(prolylglycyl), Motuporin, Dirucotide, Plitidepsin, Tigapotide, Peptide YY (3-36), Ularitide, SF1126, Glatiramer, Tifuvirtide, ABT-510, ATN-161, Telaprevir, Teverelix, Glypromate, CYT006-AngQb, NBI-6024, Pegdinetanib, Contulakin-G, Trofinetide, IRL-1620, Nelipepimut-S, Trafermin, Anamorelin, DiaPep 277, Cholecystokinin, Carfilzomib, Prezatide, Emodepside, Eftrenonacog alfa, Depreotide, Rusalatide acetate, Acetyl hexapeptide-3, Angiotensin 1-7, Rapastinel, Dusquetide, BPC-157, Acyline, Elamipretide, Oprozomib, BQ-123, Surotomycin, Solnatide, TRV-120027, OBP-801, Ipamorelin, Forigerimod, Nepadutant, Ozarelix, Davunetide, Aclerastide, Nona-arginine, Soblidotin, Relamorelin, LTX-109, Tiplimotide, LTX-315, GS-9256, Cibinetide, Rotigaptide, Alsactide, Angiotensinamide, Malacidin A, Malacidin B, Reversin 121, Murepavadin, Avexitide, Dalantercept, Eflapegrastim, Ipafricept, Beinaglutide, BPI-3016, Mibenratide, Edratide, PL-3994, Balixafortide, NNZ-2591, Zilucoplan, SER-100, VEGFR2-169, Nangibotide, Parathyroid Hormone-Related Protein 1-36, Pam2csk4, Pegcetacoplan, Tisotumab vedotin, MM3122, Atazanavir, Bradykinin, Denileukin diftitox, Peginterferon alfa-2a, Interferon alfa-n1, Erythropoietin, Salmon calcitonin, Interferon alfa-n3, Sargramostim, Peginterferon alfa-2b, Menotropins, Interferon gamma-1b, Interferon alfa-2a, Oprelvekin, Palifermin, Aldesleukin, Insulin lispro, Insulin glargine, Abciximab, Interferon beta-1a, Eptifibatide, Follitropin, Interferon beta-1b, Interferon alfacon-1, Insulin pork, Pegvisomant, Felypressin, Urofollitropin, Becaplermin, Interferon alfa-2b, Aspartame, Bleomycin, Capreomycin, Anidulafungin, Vancomycin, Caspofungin, Colistin, Dactinomycin, Colistimethate, icafungin, Pramlintide, Carbetocin, Corticotropin, Insulin aspart, Quinupristin, Virginiamycin M1, Dalfopristin, Ilomastat, N-(1-carboxy-3-phenylpropyl)phenylalanyl-alpha-asparagine, S-(2,4-dinitrophenyl)glutathione, Phosphoramidon, Terlipressin, JE-2147, N-(3-Propylcarbamoyloxirane-2-Carbonyl)-Isoleucyl-Proline, S-octylglutathione, Glutathionylspermidine, Enalkiren, gamma-Glutamylcysteine, Gallichrome, Trypanothione, Z-Pro-Prolinal, S-benzylglutathione, S-(4-nitrobenzyl)glutathione, S—(N-hydroxy-N-bromophenylcarbamoyl)glutathione, Balhimycin, S-Hexylglutathione, S-Methyl glutathione, Virginiamycin S1, Olcegepant, Vapreotide, Lucinactant, Nesiritide, Thymalfasin, G17DT, Pexelizumab, Ramoplanin, Canfosfamide, Abaloparatide, Leptin, NN344, Aminocandin, CR665, PM02734, Elafin, Interferon alfa, Cintredekin besudotox, Romiplostim, Ciliary neurotrophic factor, Regramostim, NOV-002, Corticorelin, Albinterferon Alfa-2B, Ezatiostat, Golotimod, VX-765, Fibroblast growth factor-1, IRX-2, Flovagatran, Maxy-G34, Cefilavancin, Oglufanide, PRO-542, Parathyroid hormone, Sar9, Met (O2)11-Substance P, CTCE-0214, Teicoplanin, Darinaparsin, Talabostat, Icatibant, Dalbavancin, Aviptadil, Teriparatide, Saxagliptin, Calcitonin gene-related peptide, Telavancin, Endostatin, Repifermin, Velafermin, Thrombopoietin, Labradimil, Emfilermin, Omiganan, Pasireotide, Saralasin, Viomycin, acetylleucyl-leucyl-norleucinal, Kelatorphan, DADLE, DPDPE, Tesamorelin, Brentuximab vedotin, Fusafungine, Enviomycin, Atosiban, Corticorelin ovine triflutate, Somatrem, Somatostatin, Ancestim, Methoxy polyethylene glycol-epoetin beta, Chorionic Gonadotropin (Human), Sincalide, Polaprezinc, Conestat alfa, Secretin human, Desirudin, Thiostrepton, Virginiamycin, Amphomycin, Insulin peglispro, Antithrombin III human, Epoetin delta, Eptotermin alfa, Dibotermin alfa, Carnosine, Bombesin, Endostar, Cenderitide, Birinapant, Neuropeptide Y, Danegaptide, Lampalizumab, Indium In-Ill pentetreotide, Astodrimer, Valspodar, Alanyl glutamine, Lenomorelin, Avotermin, TAK-448, Interleukin-7, Thymosin beta-4, Ibodutant, Nagrestipen, TT-232, Alisporivir, Binetrakin, Dulanermin, Muplestim, SCY-635, Elobixibat, Selepressin, Molgramostim, Pentetreotide, Sprifermin, Nerve Growth Factor, Endothelin-1, Balugrastim, Dolastatin 10, Zoptarelin doxorubicin, Somatoprim, CMX-2043, Pancreatic Polypeptide, Pimabine, Interleukin-10, Albusomatropin, Bleomycin A6, Urocortin-2, 9-(N-methyl-L-isoleucine)-cyclosporin A, Neladenoson bialanate, Lenograstim, Plecanatide, Lipegfilgrastim, Arginine glutamate, Sulglicotide, Ornipressin, Tyrothricin, Calcium pangamate, Mifamurtide, Pristinamycin, Elcatonin, Demoxytocin, Paclitaxel poliglumex, Cenegermin, Lutetium Lu 177 dotatate, Valinomycin, Basifungin, Interleukin-1 alpha, human recombinant, Basic Fibroblast Growth Factor, Nepidermin, Insulin-like growth factor II, Luteinizing hormone, Peginterferon lambda-1a, Gancotamab, Demplatin pegraglumer, Procalcitonin, Human interleukin-2, Vobarilizumab, Aspartyl-alanyl-diketopiperazine, Somatrogon, Enarodustat, Recombinant CD40-ligand, Leridistim, Somatropin pegol, Interferon alfa-2c, Bempegaldesleukin, Pegilodecakin, Tirzepatide, Gastric inhibitory polypeptide, Dasiglucagon, Bulevirtide, Thyrotropin, VM4-037, Pegbelfermin, ALT-801, Teceleukin, Tucotuzumab celmoleukin, Lorukafusp alfa, Tallimustine, Echinomycin, Atrial natriuretic peptide, Edodekin alfa, Viral Macrophage-Inflammatory Protein, Interferon Kappa, Copper oxodotreotide Cu-64, Glycylglutamine, Glycyltyrosine, Iseganan, Aldafermin, Dapirolizumab pegol, Dopastatin, Dynorphin, Gontivimab, C-peptide, Kisspeptin-10, Pegylated synthetic human c-peptide, Pexiganan, Rezafungin, Efineptakin alfa, Dialyzable leukocyte extract, Amlintide, Tengonermin, Insulin icodec, Leupeptin, or Interferon tau.

In some embodiments, the nucleic acid agent is selected from siRNA, mRNA, or a DNA oligonucleotide. In some embodiments, the siRNA is a knockout sequence. In some embodiments, the siRNA is a CRISPR guide sequence. In some embodiments, one or a plurality of CRISPR guide sequences and a Cas protein (Cas9, Casl2a, Cas13) are the therapeutic agent. In some embodiments, the mRNA is a sequence encoding all or part of a antigen protein. In some embodiments, the mRNA sequence encodes for all or a part of the SARS-CoV-2 spike (S) protein.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Materials and Methods

All DNA oligonucleotides were purchased from Integrated DNA Technologies Inc. with standard desalting unless mentioned otherwise. The strands used for dynamic reconfiguration were purified in house with denaturing PAGE gels. Lipids were purchased from Avanti Polar Lipids Inc. All other chemicals (e.g. —Sucrose, Glucose, FITC-Dextran, alpha-hemolysin etc.) were purchased from Sigma Aldrich Inc.

### Large Diameter DNA Channel Design and Assembly

The nanostructures, including the ones having the DNA channel with no lid (LGC-N), and the channel with lid (LGC), were designed de novo by Tiamat software. Assembly of these structures without cholesterol modification were done by mixing a final concentration of the M13mp18 scaffold strand at 50 nM and each of the staple strands at 5× excess concentration in 12.5 mM MgCl₂ in 1×TAE buffer (40 mM Triacetate and 1 mM EDTA, pH 8.3). The structures were folded in Life Technologies SimpliAmp thermal cycler by the following annealing protocol—10 mins consecutively at 90° C., 80° C., 70° C. and 60° C.; 20 mins consecutively at 50° C., 40° C., 30° C. and 20° C. Following the annealing the structures were stored at 4° C. until further use.

Cholesterol modified strands (SEQ ID NO: 227) were purchased from Integrated DNA Technologies Inc. where the cholesterol-tetra ethylene glycol (Chol-TEG) was covalently labelled at the 3′ end of the oligo. with PAGE purification. The strand was dissolved in water to make 100 μM solution. Immediately the solution was made into 5, 10 and 20 μl aliquots and lyophilized. This was done in order to avoid aggregation of the cholesterol modified strands in water. The DNA channels as synthesized were then incubated with 2×excess concentration of cholesterol modified strands (64 cholesterol modified positions on the structure were accounted for) at 37° C. for 12 hrs.

Sequences for construction of the LGC with open lid (LGC-O), the LGC with closed lid (LGC-C) and LGC for FRET analysis, and key sequences and other modification sequences, were set forth in Table 1 below. The LGC-O with strands for cholesterol and fluorophore modifications was prepared by using the L-core, L-chol and L-f strand sequences, the LGC-C with strands for cholesterol and fluorophore modification was made using the L-core, L-chol, L-f, PL-1, PL-2, and LL strand sequences, and the LGC-C for PRET analysis was prepared by using the L-core, PL-1, PL-2, Cy3FR and Cy5FR strand sequences.

TABLE 1
Sequences for LGC-C/O construction
Core sequences

| L-core-1 | GCGTTTTGTCGCTGAGGCTTGCTAACCGAT |
|---|---|
| | (SEQ ID NO: 1) |
| L-core-2 | TAAACAGCAACCATCGCCCACGCAAGGGAG |
| | TT |
| | (SEQ ID NO: 2) |
| L-core-3 | ATTAAGAGCTTTTGCGGGATCGTCCGCCGA |
| | CA |
| | (SEQ ID NO: 3) |
| L-core-4 | ACAACTTTTTTCTGTATGGGATACCCTCAG |
| | (SEQ ID NO: 4) |
| L-core-5 | TATCATACAGAAAACGAGAATGCAAATATC |
| | (SEQ ID NO: 5) |
| L-core-6 | TCCCCCTCACGAACCAGACCGGAAGTTTTC |
| | AAACTCCAACAG |
| | (SEQ ID NO: 6) |
| L-core-7 | GTCAATCACAGGTCTTTACCCTGATCAAAA |
| | AG |
| | (SEQ ID NO: 7) |
| L-core-8 | GCTTGAGATGGTTTTTCAACTTTAATCATT |
| | (SEQ ID NO: 8) |
| L-core-9 | TGCTGGTAAACAGG |
| | (SEQ ID NO: 9) |
| L-core-10 | ACAGTATTAAACTCATGGATTTTAAT |
| | (SEQ ID NO: 10) |
| L-core-11 | TTGAGAATCTACCGCCAATTTTCAAAGTTC |
| | CTGAT |
| | (SEQ ID NO: 11) |
| L-core-12 | AGAAGGAGTGCCCGAACGTTATTAGCC |
| | (SEQ ID NO: 12) |
| L-core-13 | AAAAACGTCCTTTATCAAAC |
| | (SEQ ID NO: 13) |
| L-core-14 | GTCAGGATGAGGTCATTTTTGCGGTTAGAA |
| | CC |
| | (SEQ ID NO: 14) |
| L-core-15 | TAAAACTACAGAAAAGCCCCAAAATTGTAA |
| | AC |
| | (SEQ ID NO: 15) |
| L-core-16 | TTCCTGTATTTGTTAAAATTCGCATACCCC |
| | GG |
| | (SEQ ID NO: 16) |
| L-core-17 | GCCGGAGAAAGATTCAAAAGGGTTAAATTT |
| | (SEQ ID NO: 17) |
| L-core-18 | GAACGCCAATCAGCTCATTTTTTATGAGTA |
| | AT |
| | (SEQ ID NO: 18) |
| L-core-19 | TATTCAACTTTTAAATGCAATGCCTTGCTC |
| | CT |
| | (SEQ ID NO: 19) |
| L-core-20 | AAAGAAGTAGACTGGATAGCGTCCACCAAT |
| | AG |
| | (SEQ ID NO: 20) |
| L-core-21 | TAATAACTTTGAC |
| | (SEQ ID NO: 21) |
| L-core-22 | GGCAGATAATAGATAATACATTTAATTGAG |
| | G |
| | (SEQ ID NO: 22) |
| L-core-23 | TAATAGGAGCCGTCTCACCAGTttttCACA |
| | CGACCAGGTGAGGCCAC |
| | (SEQ ID NO: 23) |
| L-core-24 | CCGTCGAGAGGGTTGATATCGGAACCTATT |
| | ATTTTTTCTGAAACA |
| | (SEQ ID NO: 24) |
| L-core-25 | TGAAAGTATTAAGTAGCGGGGTTTTGCTCC |
| | ACCCTCAGAACCGCCCCTCATTT |
| | (SEQ ID NO: 25) |
| L-core-26 | AACCGCCAAGAGAAGGATTAGGATAGGCTG |
| | AG |
| | (SEQ ID NO: 26) |
| L-core-27 | TCATACACGCCACCAGAACCACCACCACCC |
| | (SEQ ID NO: 27) |
| L-core-28 | AAGTACAGAAGGCACCAACCTAAGTAAGCG |
| | (SEQ ID NO: 28) |
| L-core-29 | CGGGTAAAAAGGAGTGTACTGGTAATTTTT |
| | AAGTTTTAACGG |
| | (SEQ ID NO: 29) |
| L-core-30 | AGTTTTACATCGCCAACGCttttTCA |
| | (SEQ ID NO: 30) |
| L-core-31 | GAGTAACAATACAGTAACACAGTAGGGCCA |
| | ACATGT |
| | (SEQ ID NO: 31) |
| L-core-32 | GGTCAGTGTGCCCCCTGCCTATTTGCGTAA |
| | CG |
| | (SEQ ID NO: 32) |
| L-core-33 | AACGGCTAAGACAGCATCGGAACGGACGTT |
| | AG |
| | (SEQ ID NO: 33) |
| L-core-34 | TAGAAAGGTTTTGTCGTCTTTCCACCGTAT |
| | AA |
| | (SEQ ID NO: 34) |
| L-core-35 | CATGTTACCAGAGGCTTTGAGGACAGGGTA |
| | GC |
| | (SEQ ID NO: 35) |
| L-core-36 | ATAATCATAATA |
| | (SEQ ID NO: 36) |
| L-core-37 | AAAGGGACACCTGAAAGCGTAACAAC |
| | (SEQ ID NO: 37) |
| L-core-38 | CCCTTCTGATTCTGGCCAAC |
| | (SEQ ID NO: 38) |
| L-core-39 | TAATAAGAATACCAGTATAAAGGGAGATTT |
| | TCAG |
| | (SEQ ID NO: 39) |
| L-core-40 | ATAATGGAAGGGTAAAACATTGCAAATCCA |
| | AT |
| | (SEQ ID NO: 40) |
| L-core-41 | AAAGTAAAAAACAAAATTGCTTTGAA |
| | (SEQ ID NO: 41) |
| L-core-42 | TAAGTCCTGAATTAAAATAATATCCCATCC |
| | (SEQ ID NO: 42) |
| L-core-43 | AAGAAGATTATTCATTTCAATTATTTGAAA |
| | CCGAA |
| | (SEQ ID NO: 43) |
| L-core-44 | AATGCAGAGAGCAA |
| | (SEQ ID NO: 44) |
| L-core-45 | CTAAAGGTCTATCAGGGCGATGATTAAATG |
| | (SEQ ID NO: 45) |
| L-core-46 | TCCAACGTCTTCTCCGTGGGAACAATTTTA |
| | CGGCGGATTGAC |
| | (SEQ ID NO: 46) |
| L-core-47 | CGTAATGGTAACCGTGCATCTGCCCCTGTG |
| | TG |
| | (SEQ ID NO: 47) |
| L-core-48 | ACCACCGGCCGCCACCCTCAGAGCCACCAG |
| | AG |
| | (SEQ ID NO: 48) |
| L-core-49 | CGCAGTCTAGCATTGACAGGAGGTGCCGCC |
| | AC |
| | (SEQ ID NO: 49) |
| L-core-50 | AATGAAATTAGCAAGGCCGGAATGAGGCAG |
| | (SEQ ID NO: 50) |
| L-core-51 | AATAAATCGATTGGCCTTGATATTAGTAGC |
| | AC |
| | (SEQ ID NO: 51) |
| L-core-52 | GTAGCGACAGCCAGCAAAATCACCGAAATA |
| | GC |
| | (SEQ ID NO: 52) |
| L-core-53 | CCAATAATTCTTACCGAAGCCCTTGCCATT |
| | TG |
| | (SEQ ID NO: 53) |
| L-core-54 | ATTATTTATAGAGATAACCCACAAGTTTTA |
| | ATTGAGTTAAGC |
| | (SEQ ID NO: 54) |
| L-core-55 | TTATTACGCAGTATGTTAGTTTAAGAAAAG |
| | TTTTTAAGCAGATAG |
| | (SEQ ID NO: 55) |
| L-core-56 | CCGAACAAAGTTAGGAATACCCAAAAGAAA |
| | GACACCACGGAATAAATGGTTTA |
| | (SEQ ID NO: 56) |
| L-core-57 | TGTCACAAGAAACGCAATAATAACCCAGAA |
| | GG |
| | (SEQ ID NO: 57) |
| L-core-58 | GGGTAATCTGGTTTGCCCCAGCAGCAAGCG |
| | (SEQ ID NO: 58) |
| L-core-59 | CCAGTGAGGCCCTGAGAGAGTTGCAGGCGA |
| | AA |
| | (SEQ ID NO: 59) |
| L-core-60 | AGAATTAATGATGGTGGTTCCGAATGCCCT |
| | TC |
| | (SEQ ID NO: 60) |
| L-core-61 | GGGTGCCGGAAGCATAAAGTGTATCGGCAA |
| | (SEQ ID NO: 61) |
| L-core-62 | ATAGGGTTTATAAATCAAAAGAATACACAA |
| | CA |
| | (SEQ ID NO: 62) |
| L-core-63 | ATTGCGTTATCCGCTCACAATTCCGTAGAT |
| | GG |
| | (SEQ ID NO: 63) |
| L-core-64 | TGAGCGAAAGCAAATATTTAAAACAGGAAG |
| | (SEQ ID NO: 64) |
| L-core-65 | AAGTTGGGAGCTTTCCGGCACCGCAGATCG |
| | CA |
| | (SEQ ID NO: 65) |
| L-core-66 | ATCGGCCTCAGGATTCTGGTGCCGGAAACA |
| | GGGGGATGTGCTGCACCAGTCAC |
| | (SEQ ID NO: 66) |
| L-core-67 | CATTCAGGCTGCGCAACTGAGTTTGAGGGG |
| | ATTTTCGACGACAGT |
| | (SEQ ID NO: 67) |
| L-core-68 | AAGGGAAGGAGTGTTGTTCCAGTTAGCCCG |
| | AG |
| | (SEQ ID NO: 68) |
| L-core-69 | GGCGCTGGATAGCAGCCTTTACAGTAGACG |
| | GG |
| | (SEQ ID NO: 69) |
| L-core-70 | GAACCTCAACGATTTTTTGTTTAAGTCAGA |
| | (SEQ ID NO: 70) |
| L-core-71 | CCCAGCTACGAGCGTCTTTCCAGACACAAA |
| | CA |
| | (SEQ ID NO: 71) |
| L-core-72 | TGACCCCCAGAATACACTAAAACAATGGAA |
| | AG |
| | (SEQ ID NO: 72) |
| L-core-73 | ATGACAACTTGATACCGATAGTTGCCAAAA |
| | AA |
| | (SEQ ID NO: 73) |
| L-core-74 | ATCTAAAGACAGCCCTCATAGTTACTGTAG |
| | CA |
| | (SEQ ID NO: 74) |
| L-core-75 | TAAATGAATCAACAGTTTCAGCGGGCGAAT |
| | AA |
| | (SEQ ID NO: 75) |
| L-core-76 | CTCATATAGCAAGGATAAAAATTTAAGCCT |
| | TT |
| | (SEQ ID NO: 76) |
| L-core-77 | GTGTAGGTACAGTCAAATCACCATTTAATG |
| | CC |
| | (SEQ ID NO: 77) |
| L-core-78 | TTGATAATGCATGTCAATCATATGGATCTA |
| | CA |
| | (SEQ ID NO: 78) |
| L-core-79 | GACGTTGTAAAACGACGGCCAGTGTACGCC |
| | AGCTGGCGAACAGGCAAAGCGCCA |
| | (SEQ ID NO: 79) |
| L-core-80 | ATTGTATAGAGAATCGATGAACAGTCTGGA |
| | (SEQ ID NO: 80) |
| L-core-81 | GGAATTAGACCGTCACCGACTTGATTAAAG |
| | GT |
| | (SEQ ID NO: 81) |
| L-core-82 | CATTACCACCATCGATAGCAGCACTTAGCG |
| | TC |
| | (SEQ ID NO: 82) |
| L-core-83 | TCAGGGATAGCAAGCCCAATAGGAGGAGGT |
| | TTAGTACCGCAGTACCAGGCGGAT |
| | (SEQ ID NO: 83) |
| L-core-84 | TCAGAGCTCAAAATCACCGGAAGCCATCTT |
| | (SEQ ID NO: 84) |
| L-core-85 | CCTCAGAAAACCGCCTCCCTCAGACATTTT |
| | CG |
| | (SEQ ID NO: 85) |
| L-core-86 | CCAGCGCCAAAGACAAAAGGGCGATATAAA |
| | AGAAACGCAACTGGCATGATTAAG |
| | (SEQ ID NO: 86) |
| L-core-87 | AAATTGTTATGGTCATAGCTGTTTGCTCGA |
| | AT |
| | (SEQ ID NO: 87) |
| L-core-88 | TACGAGCCTAATGAGTGAGCTAACTTCCAG |
| | TC |
| | (SEQ ID NO: 88) |
| L-core-89 | ACCGCCTGACGGGCAACAGCTGATATTAAT |
| | GA |
| | (SEQ ID NO: 89) |
| L-core-90 | GTCCACGCGCCAGGGTGGTTTTGGTTTGCG |
| | (SEQ ID NO: 90) |
| L-core-91 | AGCATCAGCTGAACCTCAAA |
| | (SEQ ID NO: 91) |
| L-core-92 | CGGAATTATCATCATAAAACCACC |
| | (SEQ ID NO: 92) |
| L-core-93 | TATCAGATGATGGTTATCATTTTGCGGAAA |
| | AAAGTTT |
| | (SEQ ID NO: 93) |
| L-core-94 | GTTTAttttACGTCAGATGAATCAATTCAT |
| | CAATACCTGAT |
| | (SEQ ID NO: 94) |
| L-core-95 | GCGGTCTTAACACCCTAAAATAttttTCTT |
| | TAGGAGCACAGGTGAG |
| | (SEQ ID NO: 95) |
| L-core-96 | AAGGTTATGCCTGCAACAGTGCCACGCTGA |
| | AAGGGAGGATT |
| | (SEQ ID NO: 96) |
| L-core-97 | CCTCAttttATCAATATCTGGTATCTAA |
| | (SEQ ID NO: 97) |
| L-core-98 | CGAACCACCAGCAGAGGCACAGACAATATT |
| | GATAGAA |
| | (SEQ ID NO: 98) |
| L-core-99 | TAAGAATACGTAGATAAAACAG |
| | (SEQ ID NO: 99) |
| L-core-100 | TACCATATCTGATAGCCAACATCGCCATTA |
| | AAAATTGGCTATTAG |
| | (SEQ ID NO: 100) |
| L-core-101 | TACCAAGTTAAAAATTATTTGCACGTTAGA |
| | ACC |
| | (SEQ ID NO: 101) |
| L-core-102 | ATTCGCCTGAGAAATAAAGAAATTGACTTC |
| | TGA |
| | (SEQ ID NO: 102) |
| L-core-103 | TGTTTGGATTATCGTAGAAACAATAACGG |
| | (SEQ ID NO: 103) |
| L-core-104 | TCTTTAATttttGCGCGAACCAAAATCGGA |
| | GGCGAA |
| | (SEQ ID NO: 104) |
| L-core-105 | TTCATAACCCTCAGAGCCACCACACCCTCA |
| | G |
| | (SEQ ID NO: 105) |
| L-core-106 | GTCATAGCCCCCTTATTAGCGTTTCCAGAG |
| | CC |
| | (SEQ ID NO: 106) |
| L-core-107 | AGACTGTAGCGCGTTTTCATCGGACGTCAC |
| | C |
| | (SEQ ID NO: 107) |
| L-core-108 | GAATTATCAGAATCAAGTTTGCCTCGTAAT |
| | CA |
| | (SEQ ID NO: 108) |
| L-core-109 | CGGAAATTATTCACAAACGTAGAAAATACA |
| | T |
| | (SEQ ID NO: 109) |
| L-core-110 | ACATAAAGCAACACATTCAACCGATGGAGG |
| | GAAGGTAAATATTGA |
| | (SEQ ID NO: 110) |
| L-core-111 | GTGTATCAACTCAACCCATGTACCGACTGA |
| | GTTTCGTCACCAGTA |
| | (SEQ ID NO: 111) |
| L-core-112 | TAATTTTTTCACGTTGAAAATCTTTTGCTA |
| | A |
| | (SEQ ID NO: 112) |
| L-core-113 | CAAACTACAACGCAAGTATAGCCCGGAATA |
| | G |
| | (SEQ ID NO: 113) |
| L-core-114 | TTCCACAGAACAACTAAAGGAATTAGTGAG |
| | AA |
| | (SEQ ID NO: 114) |
| L-core-115 | ATTAGATATCGCAAAATC |
| | (SEQ ID NO: 115) |
| L-core-116 | AAGGCTCCAAAAGGAGCCTTTAATGAATTT |
| | CT |
| | (SEQ ID NO: 116) |
| L-core-117 | GCGGGCCTGCTATCCAAGCTTGCATGCAGG |
| | TCGACTCTAGAGGAT |
| | (SEQ ID NO: 117) |
| L-core-118 | CCCCGGGTACCGATTGGGAAGGGCGATCGG |
| | T |
| | (SEQ ID NO: 118) |
| L-core-119 | GGGAAACCTGTCGTGCCAGCTGCAAAGCCT |
| | G |
| | (SEQ ID NO: 119) |
| L-core-120 | ACTTTTGCGGGAGAGTAGATTTAGTTTGAC |
| | C |
| | (SEQ ID NO: 120) |
| L-core-121 | GGAGAGGGTAGCTATTTTTGAGATGAGAAA |
| | G |
| | (SEQ ID NO: 121) |
| L-core-122 | ATTTCAACCGTTCTAGCTGATAAACAATAT |
| | GA |
| | (SEQ ID NO: 122) |
| L-core-123 | GCAAACATAACGCCAGGGTTTTCAGGCGAT |
| | T |
| | (SEQ ID NO: 123) |
| L-core-124 | AAGGCTATCAGGTCATTGCCTGAGGGTAAT |
| | CG |
| | (SEQ ID NO: 124) |
| L-core-125 | TCGTAATCGCGCTCACTGCCCGCTTCACAT |
| | TA |
| | (SEQ ID NO: 125) |
| L-core-126 | ATCGGCCAACGCGCGGGGAGAGGCTCTTTT |
| | CA |
| | (SEQ ID NO: 126) |
| L-core-127 | TATTGGGTCAATAGAAAATTCATGTTTATT |
| | T |
| | (SEQ ID NO: 127) |
| L-core-128 | ATTCCCAATTCTGCGAACGATGGCTTAGAG |
| | CttttTTAATTGCTG |
| | (SEQ ID NO: 128) |
| L-core-129 | ATGCGTTATCAT |
| | (SEQ ID NO: 129) |
| L-core-130 | CCTTAGATTTTC |
| | (SEQ ID NO: 130) |
| L-core-131 | ACGCTGAGCTATTAATTAAATCCT |
| | (SEQ ID NO: 131) |
| L-core-132 | CTTCTGACCTTG |
| | (SEQ ID NO: 132) |
| L-core-133 | AATCATAGTGAGTGAATATAAATCGTCGAA |
| | GAGTCA |
| | (SEQ ID NO: 133) |
| L-core-134 | TCTGACGTTTGAAATAAGTTAAGATTAAG |
| | (SEQ ID NO: 134) |
| L-core-135 | ATAGTGAAATTTTCCGACCGTGTGTTAAAT |
| | AAGAATATTTTATCAA |
| | (SEQ ID NO: 135) |
| L-core-136 | TTAATGCTAAAT |
| | (SEQ ID NO: 136) |
| L-core-137 | TAAGGCGATAAA |
| | (SEQ ID NO: 137) |
| L-core-138 | GACTACCTTTTCAAATAAACACCACTAGAA |
| | AAAAACTTTTTTAACC |
| | (SEQ ID NO: 138) |
| L-core-139 | TCCGGCTTTTTAATGGAATCAATATATGGT |
| | CTGAGA |
| | (SEQ ID NO: 139) |
| L-core-140 | CATAAAACAGTA |
| | (SEQ ID NO: 140) |
| L-core-141 | CATTTGCAATTT |
| | (SEQ ID NO: 141) |
| L-core-142 | ATAATTGGAATC |
| | (SEQ ID NO: 142) |
| L-core-143 | TAACAACGCTTAA |
| | (SEQ ID NO: 143) |
| L-core-144 | AATATAATGCTGTACGGTGTCTGGAAGTTT |
| | TTAGCTATATTTTCAAATTAAGC |
| | (SEQ ID NO: 144) |
| L-core-145 | GCGAGCTGAATATGCAACTAAAGTAGCTCA |
| | AC |
| | (SEQ ID NO: 145) |
| L-core-146 | ATATTCGGCTTGCTTTCGAGGTTGTATCGG |
| | T |
| | (SEQ ID NO: 146) |
| L-core-147 | TTATCAAAAAGGTGGCATCAATTTTGGGGC |
| | (SEQ ID NO: 147) |
| L-core-148 | TTATATAAGAGAAGCCTGTTTAGTATACAA |
| | A |
| | (SEQ ID NO: 148) |
| L-core-149 | ACATTTAAAATTACCTTTAGGTTGGG |
| | (SEQ ID NO: 149) |
| L-core-150 | CGCCTATATGTAAATGC |
| | (SEQ ID NO: 150) |
| Strands for cholesterol modification | |
| (64 sites) | |
| L-chol-1 | TCAAAAATTACGAGGCATAGTAAGTAACGC |
| | CAtaacaggattagcagagcgagg |
| | (SEQ ID NO: 151) |
| L-chol-2 | GTCAGAATTAGCCGGAACGAGGACCAACTT |
| | taacaggattagcagagcgagg |
| | (SEQ ID NO: 152) |
| L-chol-3 | AAAGGAATACACCAGAACGAGTAGATCGGC |
| | CTtaacaggattagcagagcgagg |
| | (SEQ ID NO: 153) |
| L-chol-4 | TGAAAGAATTCAGTGAATAAGGGCCATATT |
| | taacaggattagcagagcgagg |
| | (SEQ ID NO: 154) |
| L-chol-5 | CCTGAGTAGATTTTAGAACTCAAACTTAAA |
| | TTGGtaacaggattagcagagcgagg |
| | (SEQ ID NO: 155) |
| L-chol-6 | GGAATCGTCCAAAATAGCGAGAGGCATCAG |
| | TTtaacaggattagcagagcgagg |
| | (SEQ ID NO: 156) |
| L-chol-7 | ACATTATCCTTATGCGATTTTATTGATTAG |
| | taacaggattagcagagcgagg |
| | (SEQ ID NO: 157) |
| L-chol-8 | AATGTTTTTTGCCAGAGGGGGTACGGAACA |
| | taacaggattagcagagcgagg |
| | (SEQ ID NO: 158) |
| L-chol-9 | CTCATTATTTAATAAAACGAACTATTTTTG |
| | GGtaacaggattagcagagcgagg |
| | (SEQ ID NO: 159) |
| L-chol-10 | AATCTACGACCAGTCtaacaggattagcag |
| | agcgagg |
| | (SEQ ID NO: 160) |
| L-chol-11 | CGTGAACCGCCGTAAAGCACTAAAGGAAGA |
| | AAtaacaggattagcagagcgagg |
| | (SEQ ID NO: 161) |
| L-chol-12 | AATTTAGGTTTTCAGAGGCATTTTTGACAA |
| | GAtaacaggattagcagagcgagg |
| | (SEQ ID NO: 162) |
| L-chol-13 | ACCGGATATTCTTCCAAATCAACGTAACAA |
| | taacaggattagcagagcgagg |
| | (SEQ ID NO: 163) |
| L-chol-14 | TTTTCATGTGTGTCGAAATCCGCGTACAGA |
| | CCtaacaggattagcagagcgagg |
| | (SEQ ID NO: 164) |
| L-chol-15 | CGAGTAAATTTTAGAGTCTGTCCATATAAC |
| | GTtaacaggattagcagagcgagg |
| | (SEQ ID NO: 165) |
| L-chol-16 | GCTTTCCTCGTTTTCAGAGCGGGAGCTAAA |
| | taacaggattagcagagcgagg |
| | (SEQ ID NO: 166) |
| L-chol-17 | CGCAAGACAGAATATAAAGTACCGAAATCA |
| | ATAtaacaggattagcagagcgagg |
| | (SEQ ID NO: 167) |
| L-chol-18 | TGATGAAAttttCAAACATCAAGTTCTGTC |
| | Ctaacaggattagcagagcgagg |
| | (SEQ ID NO: 168) |
| L-chol-19 | AGACGACGACTTTTAATAAACAACATACAA |
| | TAGAtaacaggattagcagagogagg |
| | (SEQ ID NO: 169) |
| L-chol-20 | TAACCACCGATTAAAGGGATTTGTGTTTTT |
| | taacaggattagcagagcgagg |
| | (SEQ ID NO: 170) |
| L-chol-21 | GAGGCAAAAGCGATTATACCAAGCCAAGAA |
| | CGtaacaggattagcagagcgagg |
| | (SEQ ID NO: 171) |
| L-chol-22 | ACGCTAACAATTTTATCCTGAAATCGAGAA |
| | taacaggattagcagagcgagg |
| | (SEQ ID NO: 172) |
| L-chol-23 | TGCCAGTTATCAAGATTAGTTGCTTCGTAG |
| | GAtaacaggattagcagagcgagg |
| | (SEQ ID NO: 173) |
| L-chol-24 | AAATGAAATCTAAGAACGCGAGGCAAGGCT |
| | TAtaacaggattagcagagcgagg |
| | (SEQ ID NO: 174) |
| L-chol-25 | ACATAAAAAAGCGAAAGGAGCGGCTGCGCG |
| | taacaggattagcagagcgagg |
| | (SEQ ID NO: 175) |
| L-chol-26 | GGTATTAATCTTTCCTTATCATTCCGAGCC |
| | AGtaacaggattagcagagcgagg |
| | (SEQ ID NO: 176) |
| L-chol-27 | CAAGCAAGAGCATGTAGAAACCCAAAAGGT |
| | taacaggattagcagagcgagg |
| | (SEQ ID NO: 177) |
| L-chol-28 | TCCGGTATACGCGCCTGTTTATCAGTTCAG |
| | CTtaacaggattagcagagcgagg |
| | (SEQ ID NO: 178) |
| L-chol-29 | AGACCTGCCAGAATCCTGAGAATAGACAGG |
| | taacaggattagcagagcgagg |
| | (SEQ ID NO: 179) |
| L-chol-30 | ATTGCATATCCAGAACAATATCTTGCCCTt |
| | aacaggattagcagagcgagg |
| | (SEQ ID NO: 180) |
| L-chol-31 | GCAAATGAAAACAGTTTTTACAAACAATTC |
| | GACCTACATATCACTTGtaacaggattagc |
| | agagcgagg |
| | (SEQ ID NO: 181) |
| L-chol-32 | GAGTCCACAGCCGGCGAACGTGGCAATGCG |
| | CCtaacaggattagcagagcgagg |
| | (SEQ ID NO: 182) |
| L-chol-33 | ATGTTTTAAATTCGAGCTTCAAAGAATGCT |
| | TTtaacaggattagcagagcgagg |
| | (SEQ ID NO: 183) |
| L-chol-34 | AAAGGCCGGAAGCCCGAAAGACTTACCATA |
| | AAtaacaggattagcagagcgagg |
| | (SEQ ID NO: 184) |
| L-chol-35 | CAGCGAAGCAAAGCGGATTGCACTATTATA |
| | taacaggattagcagagcgagg |
| | (SEQ ID NO: 185) |
| L-chol-36 | GACGAGAATAAGGGAACCGAACTGCGCAGA |
| | CGtaacaggattagcagagcgagg |
| | (SEQ ID NO: 186) |
| L-chol-37 | GTGAATTATACAGGTAGAAAGATTCTTTTG |
| | CAtaacaggattagcagagcgagg |
| | (SEQ ID NO: 187) |
| L-chol-38 | GTTAATATGCCAGCTTTCATCAACGCCCAC |
| | TAtaacaggattagcagagcgagg |
| | (SEQ ID NO: 188) |
| L-chol-39 | TTGTTAATCAAAAATAATTCGCAATAGTAA |
| | taacaggattagcagagcgagg |
| | (SEQ ID NO: 189) |
| L-chol-40 | TTTGATAATAGAGAGTACCTTTAAAATACT |
| | GCtaacaggattagcagagcgagg |
| | (SEQ ID NO: 190) |
| L-chol-41 | ACTCCTCATGGCTTTTGATGATACTACGTA |
| | ATtaacaggattagcagagcgagg |
| | (SEQ ID NO: 191) |
| L-chol-42 | AGCTGCTCGGACAGATGAACGGTGACCTGC |
| | TCtaacaggattagcagagcgagg |
| | (SEQ ID NO: 192) |
| L-chol-43 | ACAGTTAACCTTGAGTAACAGTGCTAAAGA |
| | CTtaacaggattagcagagcgagg |
| | (SEQ ID NO: 193) |
| L-chol-44 | CAGGAGGCCACACCCGCCGCGCTTGAGAAA |
| | GGtaacaggattagcagagcgagg |
| | (SEQ ID NO: 194) |
| L-chol-45 | CCGCCGCCCTGAATTTACCGTTCCAAACGA |
| | AAtaacaggattagcagagcgagg |
| | (SEQ ID NO: 195) |
| L-chol-46 | GTCAGACCTCATTAAAGCCAGATCTTACCA |
| | taacaggattagcagagcgagg |
| | (SEQ ID NO: 196) |
| L-chol-47 | AATAGCTAAAGAGCAAGAAACAATGCCTAA |
| | TTtaacaggattagcagagcgagg |
| | (SEQ ID NO: 197) |
| L-chol-48 | AAACCGAGTGAGCGCTAATATCAGCCCAAT |
| | CCtaacaggattagcagagcgagg |
| | (SEQ ID NO: 198) |
| L-chol-49 | ATCCTGTTCTGAACACCCTGAACAAACGTC |
| | AAtaacaggattagcagagcgagg |
| | (SEQ ID NO: 199) |
| L-chol-50 | AATCCCTAACAGGGAAGCGCATAGAGAATA |
| | taacaggattagcagagcgagg |
| | (SEQ ID NO: 200) |
| L-chol-51 | GCGCATCGGATAGGTCACGTTGGTTGGAAC |
| | AAtaacaggattagcagagcgagg |
| | (SEQ ID NO: 201) |
| L-chol-52 | CTCCAGCCGTAACAACCCGTCGGAAAAGGG |
| | CGtaacaggattagcagagcgagg |
| | (SEQ ID NO: 202) |
| L-chol-53 | AACGGTACCAAGTGTAGCGGTCACGGCGCT |
| | AGtaacaggattagcagagcgagg |
| | (SEQ ID NO: 203) |
| L-chol-54 | TAATTTACGCCGTTTTTATTTTCAATTTTG |
| | CAtaacaggattagcagagcgagg |
| | (SEQ ID NO: 204) |
| L-chol-55 | ATCGGCTGACCAAGTACCGCACTCCTCATC |
| | TTtaacaggattagcagagcgagg |
| | (SEQ ID NO: 205) |
| L-chol-56 | AAACAGTTACCCTCGTTTACCAGACGATAA |
| | AAACATAAATATTCATTtaacaggattagc |
| | agagcgagg |
| | (SEQ ID NO: 206) |
| L-chol-57 | GAGATTTATACCACATTCAACTAATGCAGA |
| | TACAAGCAACACtaacaggattagcagagc |
| | gagg |
| | (SEQ ID NO: 207) |
| L-chol-58 | GCCACTACACGGAGATTTGTATCATCGATA |
| | AATAGGAAGTTTCCATTtaacaggattagc |
| | agagcgagg |
| | (SEQ ID NO: 208) |
| L-chol-59 | AGGCGCATTGGCTGACCTTCATCAAGAGTA |
| | ATCTGCGAAACAtaacaggattagcagagc |
| | gagg |
| | (SEQ ID NO: 209) |
| L-chol-60 | GCTACAGGGTACTATGGTTGCTTTGACGAG |
| | CACGTCGGAACCtaacaggattagcagagc |
| | gagg |
| | (SEQ ID NO: 210) |
| L-chol-61 | AAAAACCGGAGCCCCCGATTTAGAGCCGGG |
| | GAATATTAAAGAACGTGtaacaggattagc |
| | agagcgagg |
| | (SEQ ID NO: 211) |
| L-chol-62 | AAATAAGACCGACTTGCGGGAGGTTTGCCT |
| | TAAACAAAATAAACAGCtaacaggattagc |
| | agagcgagg |
| | (SEQ ID NO: 212) |
| L-chol-63 | ATCATTACCCCAATAGCAAGCAAATCAGAT |
| | ATAGGTTTTAGCtaacaggattagcagagc |
| | gagg |
| | (SEQ ID NO: 213) |
| L-chol-64 | GTCGAGGTATCACCCAAATCAAGTGTCTGG |
| | CCtaacaggattagcagagcgagg |
| | (SEQ ID NO: 214) |
| Strands for the fluorophore modification | |
| (5 sites) | |
| L-f-1 | ttttttttttttttttttttATAAAGCTAA |
| | TGGTCAATAACCTGTCATTCCATATAAC |
| | (SEQ ID NO: 215) |
| L-f-2 | ttttttttttttttttttttGGTTGTAAAA |
| | CATTATGACCCTGTAAT |
| | (SEQ ID NO: 216) |
| L-f-3 | ttttttttttttttttttttATACAGGCAA |
| | CTCAGAGC |
| | (SEQ ID NO: 217) |
| L-f-4 | ttttttttttttttttttttAATAAAGCGG |
| | CAAAGAATTAGCAATCTAC |
| | (SEQ ID NO: 218) |
| L-f-5 | ttttttttttttttttttttAATTTTAATA |
| | GTAGTAGCATTAACATCCAA |
| | TAAATCCTTTT |
| | (SEQ ID NO: 219) |

Lock strands

| | |
|---|---|
| Plate | CGGAGAGAAAGAGCGCTCAATTTAGACGGC |
| lockstrand-1 | AAATCAACAGTTGAGAGCCAGCA |
| (PL-1) | (SEQ ID NO: 220) |
| Plate | AGGACGTTGTCACGCAAATTAACCGTACAT |
| lockstrand-2 | TTTTTCGTTGTGTGCGCGGGTGTAGGAT |
| (PL-2) | (SEQ ID NO: 221) |
| Lid lock | CCGCGCACACAACGTGAAAACATAGCGATA |
| strand(LL) | GCTTATTTCATCTTTTTGCTCTTTCTCTCC |
| | GATAAGCTA |
| | (SEQ ID NO: 222) |

Keys

| | |
|---|---|
| Key 1 | atgctcctaATCCTACACCCGCGCACA |
| | (SEQ ID NO: 223) |
| Key 2 | gacttagaaTAGCTTATCGGAGAGAA |
| | (SEQ ID NO: 224) |
| Reverse key 1 | GCGCGGGTGTAGGATtaggagcat |
| | (SEQ ID NO: 225) |
| Reverse key 2 | CTCCGATAAGCTAttctaagtc |
| | (SEQ ID NO: 226 ) |

Modification strands

| | |
|---|---|
| Cholesterol | CCTCGCTCTGCTAATCCTGTTA/ |
| modified | 3CholTEG/ |
| strand | (SEQ ID NO: 227) |
| Cy3 modified | AAAAAAAAAAAAAAAAAAAA/3Cy3Sp/ |
| strand | (SEQ ID NO: 228) |
| (confocal) | |
| Cy5 modified | AAAAAAAAAAAAAAAAAAAA/3Cy5Sp/ |
| strand | (SEQ ID NO: 229) |
| (confocal) | |
| Cy3 FRET | TAGAAGTACGTCTGAAATGGAT/iCy3/ |
| (dye strand) | TATTTTGTAGCAATACTTCTAGAACTGG |
| (Cy3FR) | (SEQ ID NO: 230) |
| Cy5 FRET (dye | CCGCGCACACAACGTGAAAACAT/iCy5/ |
| | AGCGATAGCTTATTTCATCT |
| strand) (Cy5FR) | TTTTGCTCTTTCTCTCCGATAAGCTA |
| | (SEQ ID NO: 231) |

Sequences for construction of the LGC without lid (LGC-N) were in Table 2 below. The LGC-N with cholesterol and fluorophore modications was prepared by using the NL-core, NL-chol and NL-f strand sequences.

TABLE 2
Sequences for LGC-N construction
Core sequences

| | |
|---|---|
| NL-core-1 | GTAGCATTCCACAGTTTTGTCGTCTTTCCG |
| | GAATTGCGAATAATACGCGAAAC |
| | (SEQ ID NO: 232) |
| NL-core-2 | ACGTTGAAAGCGTAACGATCTAAAGACAGC |
| | CC |
| | (SEQ ID NO: 233) |
| NL-core-3 | TCATAGTTGAACCGCCACCCTCAGGAGACT |
| | CC |
| | (SEQ ID NO: 234) |
| NL-core-4 | ACCCTCATCTTACCGAAGCCCTAAATAGCA |
| | (SEQ ID NO: 235) |
| NL-core-5 | AGAGAGATAGAGCAAGAAACAATGTTTTAA |
| | GA |
| | (SEQ ID NO: 236) |
| NL-core-6 | AAAGTAAGGTACTCAGGAGGTTTAGGGGTT |
| | TT |
| | (SEQ ID NO: 237) |
| NL-core-7 | GTATCACCCAGATAGCCGAACAAAGTTAAG |
| | CC |
| | (SEQ ID NO: 238) |
| NL-core-8 | ATCCCAACAAAATAAACAGCCAGTTACCAG |
| | (SEQ ID NO: 239) |
| NL-core-9 | AAGGAAATATAAGTATAGCCCGCGTCGAGA |
| | (SEQ ID NO: 240) |
| NL-core-10 | AGTAAGCAGGATTAGGATTAGCGTACCGCC |
| | (SEQ ID NO: 241) |
| NL-core-11 | TAAGAGGCTAACCGCCACCCTCAGAttttGC |
| | CACCACCCTCA |
| | (SEQ ID NO: 242) |
| NL-core-12 | TGAGCCATCCAGGCGGATAAGTGCGAATAG |
| | GT |
| | (SEQ ID NO: 243) |
| NL-core-13 | TCAAGAGAGTCATACATGGCTTTTGAACAG |
| | GAGCTGAAACATGAAAG |
| | (SEQ ID NO: 244) |
| NL-core-14 | ATTGGCCTATTCACAAACAAATAAATCCTC |
| | ATTAACCGTTCC |
| | (SEQ ID NO: 245) |
| NL-core-15 | AGCCCCCTTTGGGAATTAGAGCCACACCGA |
| | CT |
| | (SEQ ID NO: 246) |
| NL-core-16 | AATCAAAATCATTACCAGAGCCACCACCGG |
| | (SEQ ID NO: 247) |
| NL-core-17 | GGGTAACGGACGGCCAGTGCCGCGAAGAGA |
| | GTTG |
| | (SEQ ID NO: 248) |
| NL-core-18 | GCCTGAGTTTGATTAGTAATAACACGACGT |
| | CGATGTTTTTACCCTAAA |
| | (SEQ ID NO: 249) |
| NL-core-19 | CTCGTCAAAGGAAGCTTGCttttATG |
| | (SEQ ID NO: 250) |
| NL-core-20 | AACCGCCTCAGGAGGTTGAGGCAGTTTAAC |
| | GG |
| | (SEQ ID NO: 251) |
| NL-core-21 | TTTTCAGGAACACTGAGTTTCGTCGAACCG |
| | AA |
| | (SEQ ID NO: 252) |
| NL-core-22 | GAATTACCAGTCAGGACGTTGGGACGGAAC |
| | AA |
| | (SEQ ID NO: 253) |
| NL-core-23 | CATTATTACAACACTATCATAACCTCAGAA |
| | AA |
| | (SEQ ID NO: 254) |
| NL-core-24 | AGTAAGAGCAGGTAGAAAGATTCAACTGGC |
| | TC |
| | (SEQ ID NO: 255) |
| NL-core-25 | TTCATCACCAGGCGCATAGGCTTCAGTTGA |
| | (SEQ ID NO: 256) |
| NL-core-26 | GATTTAGCGCCAAAAGGAATTACCCGTATA |
| | (SEQ ID NO: 257) |
| NL-core-27 | ATACATAAGAATACCACATTCAACATGAAC |
| | GG |
| | (SEQ ID NO: 258) |
| NL-core-28 | TATTCATTCTTTGAAAGAGGACAGGGAACC |
| | CA |
| | (SEQ ID NO: 259) |
| NL-core-29 | TGTACCGTGATAGCAAGCCCAATATTCGGA |
| | AC |
| | (SEQ ID NO: 260) |
| NL-core-30 | GGTCAGTGAATGCCCCCTGCCTATTAATGC |
| | AG |
| | (SEQ ID NO: 261) |
| NL-core-31 | ACCGCCACGAACCACCACCAGAGCAAGCAA |
| | AT |
| | (SEQ ID NO: 262) |
| NL-core-32 | ATTTAAATAACAGGAAGATTGTATCGAGGC |
| | AT |
| | (SEQ ID NO: 263) |
| NL-core-33 | GTGGTTCCGGCGCCAGGGTGGTTTTTCTTA |
| | TCCTAGTTTGGAACAAGAGATCCCCGG |
| | (SEQ ID NO: 264) |
| NL-core-34 | TACGCCATCGACTCTAGAGGTCCACTGCCC |
| | CAGCAGGCGAAATTCACCAGTG |
| | (SEQ ID NO: 265) |
| NL-core-35 | GGAAGGGCCGAATTCGTAATCGGTTGAGTG |
| | TTGTTCCGTTTGATG |
| | (SEQ ID NO: 266) |
| NL-core-36 | AAGAATCGAGATAGATGGTCATttttAGCT |
| | GTTTCCTGCAAAGCGCC |
| | (SEQ ID NO: 267) |
| NL-core-37 | AAGAACGGGTATTAAACCATATCAACAATA |
| | GttttATAAGTCCTG |
| | (SEQ ID NO: 268) |
| NL-core-38 | AACAAGAAAAATACCAATCAATAATCGGCA |
| | TCATTACCGCGCCCACGGTATTC |
| | (SEQ ID NO: 269) |
| NL-core-39 | CAAATCAGTACGAGCATGTAGAAAATATCC |
| | CA |
| | (SEQ ID NO: 270) |
| NL-core-40 | TCCTAATTGCCAGTAATAAGAGAACATACA |
| | TA |
| | (SEQ ID NO: 271) |
| NL-core-41 | TTTTCGAAACACCGGAATCATACGTTAAAT |
| | (SEQ ID NO: 272) |
| NL-core-42 | GACAAAAAACATATAAAAGAAACAGAGGCA |
| | (SEQ ID NO: 273) |
| NL-core-43 | TAGAAAATATATAAAGTACCGACAAttttA |
| | AGGTAAAGTAAT |
| | (SEQ ID NO: 274) |
| NL-core-44 | AAGGTGGCGGGCGACATTCAACCGATGGAG |
| | GGACAGTATGTTAGCAA |
| | (SEQ ID NO: 275) |
| NL-core-45 | GAAACGTCATGAAACCATCGATAGCAGCAC |
| | CGTAGCGCCAAA |
| | (SEQ ID NO: 276) |
| NL-core-46 | ACTTTGCTTTGACCGTTGTttttAGC |
| | (SEQ ID NO: 277) |
| NL-core-47 | CGTGAACCCAGGGCGCGTAATACTTCTAGA |
| | AGAACT |
| | (SEQ ID NO: 278) |
| NL-core-48 | GTAGCGCGGCACCATTACCATTAGAAATTA |
| | TT |
| | (SEQ ID NO: 279) |
| NL-core-49 | TCTGTCCAGCAGAACGCGCCTGTTTCTTAC |
| | CA |
| | (SEQ ID NO: 280) |
| NL-core-50 | ATACCCAACCGAGGAAACGCAATACCTAAT |
| | TT |
| | (SEQ ID NO: 281) |
| NL-core-51 | TTAACGTCGAGCGTCTTTCCAGAGAACATG |
| | TT |
| | (SEQ ID NO: 282) |
| NL-core-52 | CAGCTAATGACGACGACAATAAACAAGACT |
| | CC |
| | (SEQ ID NO: 283) |
| NL-core-53 | CATTAAAGAAGAACTGGCATGATTATAACG |
| | GA |
| | (SEQ ID NO: 284) |
| NL-core-54 | AAACCAGGTGTGAAATTGTTCCGGAAGCAT |
| | AAAATCAA |
| | (SEQ ID NO: 285) |
| NL-core-55 | AGAACAATATCACGCAAATTAACGAGCACC |
| | CGCC |
| | (SEQ ID NO: 286) |
| NL-core-56 | AGGGAAGAAAGCGAGCGGGTTAGGAGTAAA |
| | AG |
| | (SEQ ID NO: 287) |
| NL-core-57 | AAAAACGCAGTGAGGCCACCAATCAG |
| | (SEQ ID NO: 288) |
| NL-core-58 | TCACACGACCATTAAAAGGGACATTCTGGC |
| | (SEQ ID NO: 289) |
| NL-core-59 | AAATGGATTACGCCAGAATCCGGATTTTAG |
| | ACAGGAAAGTGAGCGTGCC |
| | (SEQ ID NO: 290) |
| NL-core-60 | GATTGACCTGGGATAGGTCACGTTGGTGTA |
| | GATGTTGTTAAA |
| | (SEQ ID NO: 291) |
| NL-core-61 | GACAGTATGATTCTCCGTGGGAACCATCAA |
| | AA |
| | (SEQ ID NO: 292) |
| NL-core-62 | ATTCGCATACCCCGGTTGATAACTCGTTTA |
| | (SEQ ID NO: 293) |
| NL-core-63 | ATCATATGTTAAATTTTTGTTAAATCCATT |
| | TTTATCGATGAACGGTA |
| | (SEQ ID NO: 294) |
| NL-core-64 | GTAAAACTATAGCGAGAGGCTTTTGttttCA |
| | AAAGAAGTTTT |
| | (SEQ ID NO: 295) |
| NL-core-65 | GCCAGAGGTCCAATACTGCGGAATAAGTAC |
| | GG |
| | (SEQ ID NO: 296) |
| NL-core-66 | TTCATCTTCGTGTGATAAATAAGGATTACT |
| | AG |
| | (SEQ ID NO: 297) |
| NL-core-67 | AAAAAGCCCAACATGTAATTTAGGCGCAAA |
| | GA |
| | (SEQ ID NO: 298) |
| NL-core-68 | AACAACGCTGTTTAGTATCATATGGGTTTG |
| | AA |
| | (SEQ ID NO: 299) |
| NL-core-69 | GAGTCAAAGCTTAGATTAAGACCGTTATAC |
| | (SEQ ID NO: 300) |
| NL-core-70 | AAATTCTCTTAATTGAGAATCGTCAACAGT |
| | (SEQ ID NO: 301) |
| NL-core-71 | CAGTAGGGTACCAGTATAAAGCCATTGAAA |
| | AC |
| | (SEQ ID NO: 302) |
| NL-core-72 | TCTGAGAGTTTTCCCTTAGAATCCTTGTTT |
| | GG |
| | (SEQ ID NO: 303) |
| NL-core-73 | ATTATACTATCAATATAATCCTGACTAAAA |
| | TA |
| | (SEQ ID NO: 304) |
| NL-core-74 | GGCAATTCTCTGAATAATGGAAGGCGTCGC |
| | TA |
| | (SEQ ID NO: 305) |
| NL-core-75 | GATTAGAGCTATCATCATATTCCTGttttA |
| | TTATCAGATGAT |
| | (SEQ ID NO: 306) |
| NL-core-76 | TTTTACATCGGGAGAAACAGTTAGAACCTA |
| | CttttCATATCAAAA |
| | (SEQ ID NO: 307) |
| NL-core-77 | TTATTTGCACGTAGGTTTAACGTCAGATGG |
| | GCGAATTATTCATTTAACATCAA |
| | (SEQ ID NO: 308) |
| NL-core-78 | TGAGCAAAATTGCGTAGATTTTCAAAACAG |
| | AA |
| | (SEQ ID NO: 309) |
| NL-core-79 | ATAAAGAACCAGAAGGAGCGGAATCGTCAA |
| | TA |
| | (SEQ ID NO: 310) |
| NL-core-80 | GAAACCACAAATCACCATCAATAAGGCCGG |
| | (SEQ ID NO: 311) |
| NL-core-81 | TATATTTTATTCAAAAGGGTGAGAATGATA |
| | TT |
| | (SEQ ID NO: 312) |
| NL-core-82 | CAACCGTTAACATTATCATTTTGCAGTATT |
| | AG |
| | (SEQ ID NO: 313) |
| NL-core-83 | GTTTGAGTCTAGCTGATAAATTAATAATGT |
| | GT |
| | (SEQ ID NO: 314) |
| NL-core-84 | TGACCATTCTGCGAACGAGTAGTGCCGGAG |
| | (SEQ ID NO: 315) |
| NL-core-85 | AGGGTAGCCCGAACGTTATTAAGTATTAAA |
| | (SEQ ID NO: 316) |
| NL-core-86 | CTATCAGGCTATTTTTGAGAGATCACAGTT |
| | GA |
| | (SEQ ID NO: 317) |
| NL-core-87 | TAACCTGTAGTTTCATTCCATATATTTAGA |
| | CT |
| | (SEQ ID NO: 318) |
| NL-core-88 | GGATAGCGGGGTAATAGTAAAATGTGGAGC |
| | AA |
| | (SEQ ID NO: 319) |
| NL-core-89 | CCAGACGAATAAAACGAACTAAAGAAAAAT |
| | (SEQ ID NO: 320) |
| NL-core-90 | TTCAGAAAACGATAAAAACCAAAAGCATGT |
| | CA |
| | (SEQ ID NO: 321) |
| NL-core-91 | GGAAGCAAACGAGAATGACCATAATTAAAC |
| | AG |
| | (SEQ ID NO: 322) |
| NL-core-92 | CCCCTCAAATGCTATCAAAAATCAGGTCTT |
| | CGAGCTTCAAAGCGAATTAGAGA |
| | (SEQ ID NO: 323) |
| NL-core-93 | CAGAAGCAAAGCGGATTGCCGTCATAAATA |
| | TttttTCATTGAATC |
| | (SEQ ID NO: 324) |
| NL-core-94 | ATAATTCGTCATTGCCTGAGAGTCTACAAA |
| | GG |
| | (SEQ ID NO: 325) |
| NL-core-95 | TTTCATCAAACAATTCGACAACTCTTTTAA |
| | AA |
| | (SEQ ID NO: 326) |
| NL-core-96 | CAGCAGCATTTGAGGATTTAGAGGAACAAA |
| | (SEQ ID NO: 327) |
| NL-core-97 | TCAATATCAATTGAGGAAGGTTATACGCTC |
| | AA |
| | (SEQ ID NO: 328) |
| NL-core-98 | ATAGAAAAAATAAGTTTATTTTGTCCATAT |
| | TT |
| | (SEQ ID NO: 329) |
| NL-core-99 | GATAATACAAATGAAAAATCTAAAGCCCTT |
| | GCTAGCACTAACAACTA |
| | (SEQ ID NO: 330) |
| NL-core-100 | AGCCAGCTACATTAAATGTGAGCGTAGCCA |
| | GC |
| | (SEQ ID NO: 331) |
| NL-core-101 | ACGAACCACAGAAGATAAAACAGAGGTGAG |
| | GCGGCTGAGAGC |
| | (SEQ ID NO: 332) |
| NL-core-102 | CAACAGAGAAACATCGCCATTAAAACCCTC |
| | AA |
| | (SEQ ID NO: 333) |
| NL-core-103 | CGTAAGAATTAGTCTTTAATGCGCCACAAT |
| | CA |
| | (SEQ ID NO: 334) |
| NL-core-104 | CAATAATAAACCCACAAGAATTGAGAAGCG |
| | CA |
| | (SEQ ID NO: 335) |
| NL-core-105 | ACGCTAACACAATTTTATCCTGAATATTTT |
| | GC |
| | (SEQ ID NO: 336) |
| NL-core-106 | GCCAGTTATCCAAATAAGAAACGAGCCTTT |
| | AC |
| | (SEQ ID NO: 337) |
| NL-core-107 | TTGTATCAGAAAGGAACAACTAAAAGACGT |
| | TAGTAAAT |
| | (SEQ ID NO: 338) |
| NL-core-108 | CTGACCAAACGGTCAATCATAAGGCCGGAA |
| | CG |
| | (SEQ ID NO: 339) |
| NL-core-109 | TGTACAGAAGAGTAATCTTGACAACAAAGC |
| | TG |
| | (SEQ ID NO: 340) |
| NL-core-110 | ATTATACCTTATGCGATTTTAAGACCTGAC |
| | GA |
| | (SEQ ID NO: 341) |
| NL-core-111 | GTACCTTTAATTGCTCCTTTTGATAATATC |
| | GCGTTTTAATTTACCCTGACTATT |
| | (SEQ ID NO: 342) |
| NL-core-112 | CTACGTTTTAATTTCAACTTTATGGGCTTG |
| | (SEQ ID NO: 343) |
| NL-core-113 | TTAATTAACCTTGCTTCTGTAAATATATGT |
| | GA |
| | (SEQ ID NO: 344) |
| NL-core-114 | ATAGCGATTAGTGAATTTATCAAACCGGCT |
| | TA |
| | (SEQ ID NO: 345) |
| NL-core-115 | TAAGAACGCGAGGCGTTTTAGCGATATTTT |
| | CATCGTAGGATGTCTTTCCTTATC |
| | (SEQ ID NO: 346) |
| NL-core-116 | AAGAATATTTTCAAATATATTTGAACGCGA |
| | (SEQ ID NO: 347) |
| NL-core-117 | ATACCGACCTGACCTAAATTTAATAAATGC |
| | TG |
| | (SEQ ID NO: 348) |
| NL-core-118 | GAAAACAAAATTAATTACATTTAATACAAA |
| | ATCGCGCAGAAATATACAGTAACA |
| | (SEQ ID NO: 349) |
| NL-core-119 | TGTCTGGATTTAAATATGCAACTACTGTAG |
| | CT |
| | (SEQ ID NO: 350) |
| NL-core-120 | TTCCCAATTAGATACATTTCGCAATTGGGG |
| | CG |
| | (SEQ ID NO: 351) |
| NL-core-121 | AGGTAAAGAAATGCAATGCCTGAGTCTACT |
| | AA |
| | (SEQ ID NO: 352) |
| NL-core-122 | AGACAGTGATAAAAATTTTTAGTTATTTCA |
| | (SEQ ID NO: 353) |
| NL-core-123 | TTGCCCTCGCCTGGCCCTGA |
| | (SEQ ID NO: 354) |
| NL-core-124 | AAAGCACTAAATCGGAGGGGTCGA |
| | (SEQ ID NO: 355) |
| NL-core-125 | GGGAGCCCCCGATATCACCCAAATCAAGTG |
| | CCCACTA |
| | (SEQ ID NO: 356) |
| NL-core-126 | GCGCTttttTAATGCGCCGCTATTAGAGCT |
| | TGACGAAGCCG |
| | (SEQ ID NO: 357) |
| NL-core-127 | GAGGCGGCGTATTGGAAATCGGttttCAAA |
| | ATCCCTTATGCGGGGA |
| | (SEQ ID NO: 358) |
| NL-core-128 | CAGCAttttAGCGGTCCACGCTAGCTGA |
| | (SEQ ID NO: 359) |
| NL-core-129 | AGCTGCATTAATGAACTGGGGTGCCTAAT |
| | GCACACAA |
| | (SEQ ID NO: 360) |
| NL-core-130 | AAGTGTAAAGCTCGGCCAACGC |
| | (SEQ ID NO: 361) |
| NL-core-131 | CGGGCGCTACACTGCCCTCCAGTCGGGAAA |
| | CCTGTCTAACTCACA |
| | (SEQ ID NO: 362) |
| NL-core-132 | AGCGGGAGCTGGGCGCTGGCAAGTGTAAAG |
| | GAG |
| | (SEQ ID NO: 363) |
| NL-core-133 | GCTTTCCTCTCACGCTGCGCGTAACAGAAA |
| | GGA |
| | (SEQ ID NO: 364) |
| NL-core-134 | GCGAACGTGGCGCACCACACGTATAACGT |
| | (SEQ ID NO: 365) |
| NL-core-135 | TTAATTGCttttGTTGCGCTAAACAGGAGA |
| | TTAAAG |
| | (SEQ ID NO: 366) |
| NL-core-136 | GAAAACTATATAGAAGGCTTATCATAGCAA |
| | G |
| | (SEQ ID NO: 367) |
| NL-core-137 | ATGCAAATCCAATCGCAAGACAAATAGTTA |
| | AT |
| | (SEQ ID NO: 368) |
| NL-core-138 | GGTTGGGTTATATAACTATATGTGCTGAGA |
| | A |
| | (SEQ ID NO: 369) |
| NL-core-139 | GTGAATAAACTACCTTTTTAACCTATCATA |
| | GG |
| | (SEQ ID NO: 370) |
| NL-core-140 | TACATAAATCAATATAACGGATTCGCCTGA |
| | T |
| | (SEQ ID NO: 371) |
| NL-core-141 | TGCTTTGACAAGTCAATTTCATTTGACCTT |
| | TTTTAATGGAAACAG |
| | (SEQ ID NO: 372) |
| NL-core-142 | AACAAGCAGTTTTACCTCCCGACTTGAGGT |
| | TTTGAAGCCTTAAAT |
| | (SEQ ID NO: 373) |
| NL-core-143 | AGAGAGAATAACATAAAAACAGGTATTATT |
| | T |
| | (SEQ ID NO: 374) |
| NL-core-144 | CAAGATTAGTTGCAGTACCGCACTCATCGA |
| | G |
| | (SEQ ID NO: 375) |
| NL-core-145 | ACCCAGCTAAAAATGAAAATAGCATTTTTT |
| | GT |
| | (SEQ ID NO: 376) |
| NL-core-146 | CGAAGGGGCAAAAGAATACACTTGATAAAG |
| | TCGAAATCCGCGACCTGCT |
| | (SEQ ID NO: 377) |
| NL-core-147 | TCAGCGGAGAATATCGCCAAAACACTCATC |
| | TTTGAACGG |
| | (SEQ ID NO: 378) |
| NL-core-148 | TTAGACGGGAGAATTAACTGAACACTAATA |
| | TC |
| | (SEQ ID NO: 379) |
| NL-core-149 | AGGAGCCTAATCTCCAAAAAAAAGATTTTT |
| | TC |
| | (SEQ ID NO: 380) |
| NL-core-150 | AAGCCCGACTTCAAAGAGGTCATTTGGATG |
| | GCTTAGAGCTTAATT |
| | (SEQ ID NO: 381) |
| NL-core-151 | GCTGAATATAATGATCAAAAAGATTAAGAG |
| | G |
| | (SEQ ID NO: 382) |
| NL-core-152 | CGAGCTGAAAAGGTGGCATCAATATTTAGT |
| | T |
| | (SEQ ID NO: 383) |
| NL-core-153 | CCATGTTACTTAGAAACAACTTTCAACAGT |
| | T |
| | (SEQ ID NO: 384) |
| NL-core-154 | CTCATTCAGTGAATAAGGCTTGCGGCTGAC |
| | C |
| | (SEQ ID NO: 385) |
| NL-core-155 | AGGCGCAGACCCAAATCAACGTAAGAACCG |
| | GA |
| | (SEQ ID NO: 386) |
| NL-core-156 | AGATGGTACTCCAACAGGTCAGGACCAGAC |
| | C |
| | (SEQ ID NO: 387) |
| NL-core-157 | GAAACACCAGAACGAGTAGTAAATATCATT |
| | GT |
| | (SEQ ID NO: 388) |
| NL-core-158 | CAACATGTTTAGCTATATTTTCATATGGTC |
| | AA |
| | (SEQ ID NO: 389) |
| NL-core-159 | TAGTAGTAGCATTGGGAGAAGCCTAACCCT |
| | CA |
| | (SEQ ID NO: 390) |
| NL-core-160 | ACGCAAGAGAAGATGATGAAACACAATTAC |
| | C |
| | (SEQ ID NO: 391) |
| NL-core-161 | TATATTCGGTCGCTACAACAGAGGTGAATT |
| | TCTTAAGCTCCAAA |
| | (SEQ ID NO: 392) |
| NL-core-162 | CGCTTTTGTCATGAGGAACCGATAGTTGC |
| | (SEQ ID NO: 393) |
| NL-core-163 | GAAAGACAGCGGCTACAGAGGCTTTGAATG |
| | CCACTA |
| | (SEQ ID NO: 394) |
| NL-core-164 | TTCTGTATGGGATTTTGCTACCAGTACAAA |
| | CttttTACAACGCCT |
| | (SEQ ID NO: 395) |

Strands for cholesterol modification
(64 sites)

| | |
|---|---|
| NL-chol-1 | GCTCAGTACGCAGTCTCTGAATTTAAGCCA |
| | GAtaacaggattagcagagcgagg |
| | (SEQ ID NO: 396) |
| NL-chol-2 | GGGTTGAGTGAATTATCACCGTGCAAAATC |
| | taacaggattagcagagcgagg |
| | (SEQ ID NO: 397) |
| NL-chol-3 | ATGGAAAGTATTAGCGTTTGCCATATTAAG |
| | TTtaacaggattagcagagcgagg |
| | (SEQ ID NO: 398) |
| NL-chol-4 | ACCAGTATTTTCATCGGCATTTCATCACTT |
| | taacaggattagcagagcgagg |
| | (SEQ ID NO: 399) |
| NL-chol-5 | AAGGGGGATGttttTGCTGCAAGGCGCTTT |
| | TCATtaacaggattagcagagcgagg |
| | (SEQ ID NO: 400) |
| NL-chol-6 | CTATTATTTGTACTGGTAATAAGTGTCAGA |
| | CGtaacaggattagcagagcgagg |
| | (SEQ ID NO: 401) |
| NL-chol-7 | GCATTGACCCTCAGAGCCGCCATTCGCTAT |
| | taacaggattagcagagcgagg |
| | (SEQ ID NO: 402) |
| NL-chol-8 | AACAGTTCCTTGAGTAACAGTGCGCCGCCA |
| | taacaggattagcagagcgagg |
| | (SEQ ID NO: 403) |
| NL-chol-9 | CGCCACCACCTCAGAGCCACCACCAACTGT |
| | TGtaacaggattagcagagcgagg |
| | (SEQ ID NO: 404) |
| NL-chol-10 | GCCCCAAATGTAAACGTTAATATTCTCAGA |
| | GCtaacaggattagcagagcgagg |
| | (SEQ ID NO: 405) |
| NL-chol-11 | GTACCGAGCTGATCGGTGCGGGCCTCCCCT |
| | CAGAtaacaggattagcagagcgagg |
| | (SEQ ID NO: 406) |
| NL-chol-12 | CAAACTATttttCGGCCTTGCTGGATCAGT |
| | AGtaacaggattagcagagcgagg |
| | (SEQ ID NO: 407) |
| NL-chol-13 | CGACAGAATCATTGCCTTTAGCGTCAGACT |
| | taacaggattagcagagcgagg |
| | (SEQ ID NO: 408) |
| NL-chol-14 | TTATTACGAGGTAAATATTGACGGCAAGGC |
| | CGtaacaggattagcagagcgagg |
| | (SEQ ID NO: 409) |
| NL-chol-15 | ATTCGCCAttttTTCAGGCTGCGCGGCGCA |
| | TCtaacaggattagcagagcgagg |
| | (SEQ ID NO: 410) |
| NL-chol-16 | GTAACCGTGCATTCAGTTTGAGGGGACGAC |
| | taacaggattagcagagcgagg |
| | (SEQ ID NO: 411) |
| NL-chol-17 | AGTCTGTCCATTACCGCCAGCCATTCCTGA |
| | AAGtaacaggattagcagagcgagg |
| | (SEQ ID NO: 412) |
| NL-chol-18 | TGAGAAGTttttGTTTTTATAATCTCATGG |
| | Ataacaggattagcagagcgagg |
| | (SEQ ID NO: 413) |
| NL-chol-19 | AATACCTACAttttTTTTGACGCTCATTCA |
| | CCAGtaacaggattagcagagcgagg |
| | (SEQ ID NO: 414) |
| NL-chol-20 | CCCGTCGCGGCCTCAGGAAGATGGTGCCGG |
| | taacaggattagcagagcgagg |
| | (SEQ ID NO: 415) |
| NL-chol-21 | ACAAGAGATAACCAATAGGAACGCAAACGG |
| | CGtaacaggattagcagagcgagg |
| | (SEQ ID NO: 416) |
| NL-chol-22 | CACCACGGTTCATATGGTTTACCATATTTT |
| | TGtaacaggattagcagagcgagg |
| | (SEQ ID NO: 417) |
| NL-chol-23 | TGAAAGGTGGTCAGTTGGCAAAGAACTGAT |
| | taacaggattagcagagcgagg |
| | (SEQ ID NO: 418) |
| NL-chol-24 | TCTTTAGGGAACCTCAAATATCAAAATACC |
| | GAtaacaggattagcagagcgagg |
| | (SEQ ID NO: 419) |
| NL-chol-25 | ACTTTACACTGCAACAGTGCCACGTCAGTA |
| | TTtaacaggattagcagagcgagg |
| | (SEQ ID NO: 420) |
| NL-chol-26 | TCCTTTGCGTCTGGCCTTCCTGAGTAACAA |
| | taacaggattagcagagcgagg |
| | (SEQ ID NO: 421) |
| NL-chol-27 | AATGGCTATACGTGGCACAGACAATAATAT |
| | CCtaacaggattagcagagcgagg |
| | (SEQ ID NO: 422) |
| NL-chol-28 | AGCCCTAATAGAACCCTTCTGAGCAACAGG |
| | taacaggattagcagagcgagg |
| | (SEQ ID NO: 423) |
| NL-chol-29 | AACACCGCTATTTACATTGGCAGAATCGTC |
| | TGtaacaggattagcagagcgagg |
| | (SEQ ID NO: 424) |
| NL-chol-30 | GGTGCCGTAAACCGTCTATCAGGGTGTAAA |
| | ACCCAGGGTTTTCCCAGTTCGGTCATtaac |
| | aggattagcagagcgagg |
| | (SEQ ID NO: 425) |
| NL-chol-31 | AGACGGGCAACGGTTTATTAAAGAACGTGG |
| | ACCTGCAGGGCTGGCGAtaacaggattagc |
| | agagcgagg |
| | (SEQ ID NO: 426) |
| NL-chol-32 | CATACGAGATCCGCTCACAATTCCGGTTCC |
| | GGCACCGCTTCTCGCACTCCtaacaggatt |
| | agcagagcgagg |
| | (SEQ ID NO: 427) |
| NL-chol-33 | TCATAGTTGAACCGCCACCCTCAGGAGACT |
| | CCtaacaggattagcagagcgagg |
| | (SEQ ID NO: 428) |
| NL-chol-34 | AAAGTAAGGTACTCAGGAGGTTTAGGGGTT |
| | TTtaacaggattagcagagcgagg |
| | (SEQ ID NO: 429) |
| NL-chol-35 | AAGGAAATATAAGTATAGCCCGCGTCGAGA |
| | taacaggattagcagagcgagg |
| | (SEQ ID NO: 430) |
| NL-chol-36 | AGCCCCCTTTGGGAATTAGAGCCACACCGA |
| | CTtaacaggattagcagagcgagg |
| | (SEQ ID NO: 431) |
| NL-chol-37 | AACCGCCTCAGGAGGTTGAGGCAGTTTAAC |
| | GGtaacaggattagcagagcgagg |
| | (SEQ ID NO: 432) |
| NL-chol-38 | CATTATTACAACACTATCATAACCTCAGAA |
| | AAtaacaggattagcagagcgagg |
| | (SEQ ID NO: 433) |
| NL-chol-39 | GATTTAGCGCCAAAAGGAATTACCCGTAT |
| | Ataacaggattagcagagcgagg |
| | (SEQ ID NO: 434) |
| NL-chol-40 | TGTACCGTGATAGCAAGCCCAATATTCGGA |
| | ACtaacaggattagcagagcgagg |
| | (SEQ ID NO: 435) |
| NL-chol-41 | ACCGCCACGAACCACCACCAGAGCAAGCAA |
| | ATtaacaggattagcagagcgagg |
| | (SEQ ID NO: 436) |
| NL-chol-42 | TCCTAATTGCCAGTAATAAGAGAACATACA |
| | TAtaacaggattagcagagcgagg |
| | (SEQ ID NO: 437) |
| NL-chol-43 | GTAGCGCGGCACCATTACCATTAGAAATTA |
| | TTtaacaggattagcagagcgagg |
| | (SEQ ID NO: 438) |
| NL-chol-44 | CAGCTAATGACGACGACAATAAACAAGACT |
| | CCtaacaggattagcagagcgagg |
| | (SEQ ID NO: 439) |
| NL-chol-45 | GACAGTATGATTCTCCGTGGGAACCATCAA |
| | AAtaacaggattagcagagcgagg |
| | (SEQ ID NO: 440) |
| NL-chol-46 | AAAAAGCCCAACATGTAATTTAGGCGCAAA |
| | GAtaacaggattagcagagcgagg |
| | (SEQ ID NO: 441) |
| NL-chol-47 | AAATTCTCTTAATTGAGAATCGTCAACAGT |
| | taacaggattagcagagcgagg |
| | (SEQ ID NO: 442) |
| NL-chol-48 | ATTATACTATCAATATAATCCTGACTAAAA |
| | TAtaacaggattagcagagcgagg |
| | (SEQ ID NO: 443) |
| NL-chol-49 | GAAACCACAAATCACCATCAATAAGGCCGG |
| | taacaggattagcagagcgagg |
| | (SEQ ID NO: 444) |
| NL-chol-50 | CAACCGTTAACATTATCATTTTGCAGTATT |
| | AGtaacaggattagcagagcgagg |
| | (SEQ ID NO: 445) |
| NL-chol-51 | AGGGTAGCCCGAACGTTATTAAGTATTAAA |
| | taacaggattagcagagcgagg |
| | (SEQ ID NO: 446) |
| NL-chol-52 | GGATAGCGGGGTAATAGTAAAATGTGGAGC |
| | AAtaacaggattagcagagcgagg |
| | (SEQ ID NO: 447) |
| NL-chol-53 | TTCAGAAAACGATAAAAACCAAAAGCATGT |
| | CAtaacaggattagcagagcgagg |
| | (SEQ ID NO: 448) |
| NL-chol-54 | AGCCAGCTACATTAAATGTGAGCGTAGCCA |
| | GCtaacaggattagcagagcgagg |
| | (SEQ ID NO: 449) |
| NL-chol-55 | CAACAGAGAAACATCGCCATTAAAACCCTC |
| | AAtaacaggattagcagagcgagg |
| | (SEQ ID NO: 450) |
| NL-chol-56 | CGTAAGAATTAGTCTTTAATGCGCCACAAT |
| | CAtaacaggattagcagagcgagg |
| | (SEQ ID NO: 451) |
| NL-chol-57 | TCAAGAGAGTCATACATGGCTTTTGAACAG |
| | GAGCTGAAACATGAAAGtaacaggattagc |
| | agagcgagg |
| | (SEQ ID NO: 452) |
| NL-chol-58 | ATTGGCCTATTCACAAACAAATAAATCCTC |
| | ATTAACCGTTCCtaacaggattagcagagc |
| | gagg |
| | (SEQ ID NO: 453) |
| NL-chol-59 | AAGGTGGCGGGCGACATTCAACCGATGGAG |
| | GGACAGTATGTTAGCAAtaacaggattagc |
| | agagcgagg |
| | (SEQ ID NO: 454) |
| NL-chol-60 | GAAACGTCATGAAACCATCGATAGCAGCAC |
| | CGTAGCGCCAAAtaacaggattagcagagc |
| | gagg |
| | (SEQ ID NO: 455) |
| NL-chol-61 | GATTGACCTGGGATAGGTCACGTTGGTGTA |
| | GATGTTGTTAAAtaacaggattagcagagc |
| | gagg |
| | (SEQ ID NO: 456) |
| NL-chol-62 | ATCATATGTTAAATTTTTGTTAAATCCATT |
| | TTTATCGATGAACGGTAtaacaggattagc |
| | agagcgagg |
| | (SEQ ID NO: 457) |
| NL-chol-63 | GATAATACAAATGAAAAATCTAAAGCCCTT |
| | GCTAGCACTAACAACTAtaacaggattagc |
| | agagcgagg |
| | (SEQ ID NO: 458) |
| NL-chol-64 | ACGAACCACAGAAGATAAAACAGAGGTGAG |
| | GCGGCTGAGAGCtaacaggattagcagagc |
| | gagg |
| | (SEQ ID NO: 459) |

Strands for fluorophore modification
(5 sites)

| | |
|---|---|
| NL-f-1 | GCCGACAATGGAGGCTTGCAGGGAGTTAAA |
| | GGCtttttttttttttttttttt |
| | (SEQ ID NO: 460) |
| NL-f-2 | TGCTTTCACCATCGCttttttttttttttt |
| | ttttt |
| | (SEQ ID NO: 461) |
| NL-f-3 | ATAGCTAGAGGGTAATTGAGCGCCCTGAAC |
| | AAAGTCATTAATTGtttttttttttttttt |
| | tttt |
| | (SEQ ID NO: 462) |
| NL-f-4 | AAAGTACACAGCGATTATACCAAGACAGCT |
| | TGATAGTTTCCATTAACCCCACGGAGATtt |
| | tttttttttttttttttt |
| | (SEQ ID NO: 463) |
| NL-f-5 | GTAAAATACGTAGGACTAAAGACTTTTCGG |
| | GATCGTCACCCTCAGCAGCttttttttttt |
| | ttttttttt |
| | (SEQ ID NO: 464) |

### Native Agarose Gel Electrophoresis

For characterizing the formation of the pore, a 1.5% o agarose gel was casted 1×TAE-Mg buffer (20 mM Tris base, 10 mM acetic acid, 0.5 mM EDTA, 12.5 mM Mg(OAc)₂, pH 8.3). The gel was run at 120V in ice-water bath for 1.5h. Sybr green stain was added with the sample which was used for the gel imaging. The pores, optionally incubated with SUVs, were analyzed using 1.5% agarose gel electrophoresis in 0.5×TAE buffer (20 mM Tris base, 10 mM acetic acid, 0.5 mM EDTA, pH 8.3) at 65 V in ice-water bath for 1.5 h.

### Atomic Force Microscopy (AFM)

Samples were prepared for AFM imaging as described. A 3 μl annealed sample was deposited onto a freshly cleaved mica surface (Ted Pella) and 60 μl 1×TAE-Mg²⁺ buffer was added immediately to the sample. After about 30 sec, 3 μl NiCl₂ (25 mM) was added. An extra 60 μl of the same buffer was deposited on the AFM tip. AFM imaging was performed in the ‘ScanAsyst mode in fluid’ on the Dimension FastScan, Bruker with the Scanasyst-Fluid⁺ tips from Bruker.

### Transmission Electron Microscopy (TEM)

Samples for the TEM were prepared as described. Ten μL of the purified sample was added to a plasma treated negative stain carbon B type grid for 10 mins and wicked off. The sample was first quickly and then for 10 sec stained with 2% uranyl formate or 1% uranyl acetate with 2 mM NaOH. The stain buffer was then wicked off and the grid air dried for 20 mins. The grid was then imaged using Philips TM 12 TEM operated at 120 kV at 33000× to 80000× magnification.

### Preparation of Small Unilamellar Vesicles (SUVs)

A lipid solution (DOPC/DOPE=7:3, 10 mg/mL in chloroform) dispensed in a 2 mL-glass vial was blown dry with argon airflow. The dried lipid film was then suspended in 50 mM HEPES (pH 7.6) supplemented with 500 mM NaCl, and treated by sonication for 30 min.

### Preparation of Giant Unilamellar Vesicles (GUVs) for Confocal Measurements

The GUVs were prepared by inverted emulsion method (Krishnan, S. et al. Molecular transport through large-diameter DNA nanopores. Nat. Commun. 7, 12787, (2016)). POPC (150 μL, 10 mM) in chloroform was added to a 1 mL glass vial, the solvent was removed under vacuum and rotation using a Buchi rotary evaporator set at high vacuum for at least 30 minutes. The thin film generated was resuspended in mineral oil (150 μL) by vortexing and sonicating for 10 minutes. Twenty-five μL of inner solution (IS, the solution that would be encapsulated inside GUVs) containing ˜435 mOsm/kg sucrose was added to the mineral oil. A water-in-oil emulsion was created by suspending the IS into the mineral oil by pipetting up and down for ˜10 times followed by vortexing at highest speed for ˜30 seconds and sonicating for 10 minutes at room temperature. This emulsion was then carefully added to the top of 1 mL external solution (ES, the solution to be kept outside the GUVs) containing ˜435 mOsm/kg glucose in a plastic microcentrifuge tube. The osmolarities of the IS and ES were measured by an osmometer (Advanced Instruments Model 3320 Osmometer 2996) and balanced properly such that the osmolarity difference was less than 20 mOsm/kg. The GUVs were generated by centrifuging at 21 k×g at 4° C. for 15 minutes. The mineral oil top layer and most of the sucrose layer (˜900 μL) were carefully removed by pipettor, every time using a fresh tip. The remaining solution containing the pelleted vesicles was gently mixed with a pipettor, then transferred to a clean plastic vial leaving a small quantity to avoid contamination of remaining trace amount of mineral oil to the GUVs.

### Confocal Dye/Protein Influx Assay

For the confocal assay, samples were prepared as follows. Three μl of GUV solution was mixed with 20 nM cy3/cy5 labelled-nanopores and 2 μM dye (atto 633) or protein (GFP). The total solution was made up to 30 μl by maintaining the osmolality balance of the inside and outside of the GUVs using a buffer containing 1M HEPES and 100 mM NaCl. This solution was added to an ibidi μ-Slide 18 Well—flat slide. The slide was then centrifuged at 1000×g for 10 mins to make sure that the GUVs were settled at the bottom of the slide. Then GUVs were then imaged on Nikon C2 Laser Scanning Confocal microscope at 40× magnification, 1.3 numerical aperture (NA) using a humidity chamber maintained at 32° C. (dye)/37° C. (GFP). Images were taken over 3 hrs at multiple points.

### Single-Channel Current Recordings

For planar lipid bilayer electrophysiological current measurements, integrated chip-based, parallel bilayer recording setups (Orbit 16 and Orbit Mini; Nanion Technologies, Munich, Germany) with multielectrode-cavity-array (MECA) chips (IONERA, Freiburg, Germany) were used (Lanphere, C. et al. Design, assembly, and characterization of membrane-spanning DNA nanopores. Nat. Proto. 16, 86-130, (2020)). Bilayers were formed of DPhPC lipid dissolved in octane (10 mg/mL). The electrolyte solution was 1M KCl and 10 mM HEPES, pH 7.6. For pore insertion, a 2:1 DNA nanopore and 0.5% OPOE (n-octyloligooxyethylene, in 1M KCl, 10 mM HEPES, pH 7.6) was added to the cis chamber. Successful incorporation was observed by detecting current steps. Current traces were not Bessel-filtered and acquired at 10 kHz, using Element Data Recorder software (Element s.r.l., Italy). Single-channel analysis was performed using Clampfit (Molecular Devices, Sunnyvale, CA, USA).

### LGC Scheme Creation and Simulation

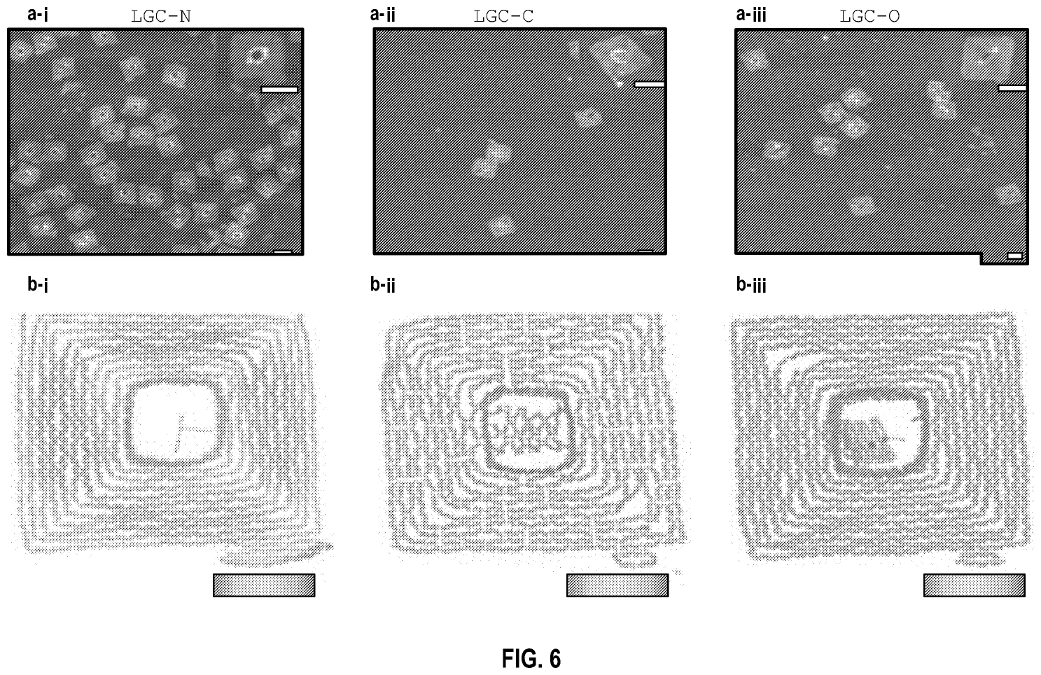

The LGC schemes were generated by converting the original Tiamat structures to .pdb structures using the TacoxDNA webserver (Suma, A. et al. Tacoxdna: A user-friendly web server for simulations of complex DNA structures, from single strands to origami. J. Comput. Chem. 40, 2586-2595, (2019)) and UCSF chimera molecular visualization tool (Pettersen, E. F. et al. Ucsf chimera? A visualization system for exploratory research and analysis. J. Comput. Chem. 25, 1605-1612, (2004)). The simulations shown in FIG. 6b were generated using oxDNA simulations in oxDNA.org (Šulc, P. et al. Design, optimization and analysis of large DNA and ma nanostructures through interactive visualization, editing and molecular simulation. Nucleic Acids Res. 48, e72-e72, (2020)).

### Kinetics of Lid Opening by FRET

The key mediated opening of the LGC is an intermolecular reaction, and so it was analyzed as a second order reaction. For the reaction shown below, the rate constant of the forward reaction was k. Once again, the backward reaction can be neglected because the free energy of hybridization precludes loss of the displacement strand.

$${Lid}_{closed} + Key \to {Lid}_{open} \qquad ( 1 )$$

At any given time point t, the rate of the reaction can be expressed as—

$$r = - \frac{d [ C ]}{dt} = \frac{d [ P ]}{dt} = {{k [ C ]}_{t} [ F ]}_{t} \qquad ( 2 )$$

where at any given time point t, [C]ₜ=concentration of Lid_{closed}, and [F]ₜ=concentration of Key.

Now, if [C]₀=concentration of Lid_{closed} at t=0 and [P]=concentration of the product, i.e., Lidₒₚₑₙ at time t, then, at any given point the total amount of closed form and the open form of the Lid was the same as the starting concentration of closed form, i.e., [C]₀.

Therefore,

$${[ C ]}_{t} + [ P ] = {[ C ]}_{0} , i . e . , {[ C ]}_{t} = {[ C ]}_{0} - [ P ] , \qquad ( 3 )$$

and similarly, for the target—

$${[ F ]}_{t} + [ P ] = {[ F ]}_{0} , i . e . , {[ F ]}_{t} = {[ F ]}_{0} - [ P ] . \qquad ( 4 )$$

Now, replacing the value of [C]ₜ and [F]ₜ from equation (2):

$$\frac{d [ P ]}{dt} = k ( {[ C ]}_{0} - [ P ] ) ( {[ F ]}_{0} - [ P ] ) \qquad ( 5 )$$

If we started with n fold of the target compared to the Lid_{closed}, then

$${[ F ]}_{0} = {n [ C ]}_{0} . \qquad ( 6 )$$

Hence, replacing the value of [F]₀=n[C]₀ in equation (5) and integrating from t=0 to t,

$${\int }_{0}^{t} kdt = {\int }_{0}^{[ P ]} \frac{d [ P ]}{( {[ C ]}_{0} - [ P ] ) ( {n [ C ]}_{0} - [ P ] )} . \qquad ( 7 )$$

Now, by simplifying [C]₀=C and [P]=x, we got—

$${\int }_{0}^{[ P ]} \frac{d [ P ]}{( {[ C ]}_{0} - [ P ] ) ( {n [ C ]}_{0} - [ P ] )} = {\int }_{0}^{x} \frac{dx}{( C - x ) ( n \cdot C - x )} \qquad ( 8 )$$

$$\frac{1}{( C - x ) ( n \cdot C - x )} = \frac{A}{( C - x )} + \frac{B}{( n \cdot C - x )} = \frac{( A \cdot n + B ) C - ( A + B ) x}{( C - x ) ( n \cdot C - x )} .$$

Therefore, (A.n+B)C−(A+B)x=1.

Therefore, A+B=0; (A.n+B)C=1,

$$A = - B = \frac{1}{( n - 1 ) C} .$$

Hence, from equation (7) and (8),

$${\int }_{0}^{[ P ]} \frac{d [ P ]}{( {[ C ]}_{0} - [ P ] ) ( {n [ C ]}_{0} - [ P ] )} = \frac{1}{( n - 1 ) C} ( {\int }_{0}^{x} \frac{dx}{( C - x )} - {\int }_{0}^{x} \frac{dx}{( n \cdot C - x )} ) = \frac{1}{( n - 1 ) C} ( {[ - ln ( C - x ) ]}_{0}^{x} - {[ - ln ( n \cdot C - x ) ]}_{0}^{x} ) = \frac{1}{( n - 1 ) C} ( ln C - ln ( C - x ) + ln ( n \cdot C - x ) - ln ( n \cdot C ) ) . \qquad ( 9 )$$

Putting back the values [C]₀=C and [P]=x in equations (7) and (9), we got—

$$\frac{1}{{( n - 1 ) [ C ]}_{0}} ( ln \vert {[ C ]}_{0} - ln ( {[ C ]}_{0} - [ P ] ) + ln ( n \cdot {[ C ]}_{0} - [ P ] ) - ln ( n \cdot {[ C ]}_{0} ) ) = kt$$

$$( {ln [ C ]}_{0} - ln ( {[ C ]}_{0} - [ P ] ) + ln ( {n [ C ]}_{0} - [ P ] ) - ln ( {n [ C ]}_{0} ) ) = {kt ( n - 1 ) [ C ]}_{0} .$$

Replacing [C]₀−[P]=[C]ₜ from equation (3),

$$( {ln [ C ]}_{0} - l \vert {n [ C ]}_{t} + ln ( {n [ C ]}_{0} - [ P ] ) - ln ( {n [ C ]}_{0} ) = {kt ( n - 1 ) [ C ]}_{0} \qquad ( 10 )$$

$$ln \frac{{n [ C ]}_{0} - [ P ]}{{[ C ]}_{t}} - ln \frac{{n [ C ]}_{0}}{{[ C ]}_{0}} = {kt ( n - 1 ) [ C ]}_{0} .$$

Putting [P]=[C]₀−[C]ₜ into equation (10),

$$ln \frac{{( n - 1 ) [ C ]}_{0} + {[ C ]}_{t}}{{[ C ]}_{t}} = ln ( n ) + {kt ( n - 1 ) [ C ]}_{0} \qquad ( 11 )$$

$$ln [ \frac{{( n - 1 ) [ C ]}_{0}}{{[ C ]}_{t}} + 1 ] = ln ( n ) + {kt ( n - 1 ) [ C ]}_{0}$$

$$\frac{{( n - 1 ) [ C ]}_{0}}{{[ C ]}_{t}} = exp [ ln ( n ) + {kt ( n - 1 ) [ C ]}_{0} ] - 1 = exp [ ln ( n ) ] \cdot exp [ {kt ( n - 1 ) [ C ]}_{0} ] - 1$$

$$\frac{{( n - 1 ) [ C ]}_{0}}{{[ C ]}_{t}} = exp [ ln ( n ) ] \cdot exp [ {kt ( n - 1 ) [ C ]}_{0} ] - 1 = n \cdot exp [ {kt ( n - 1 ) [ C ]}_{0} ] - 1$$

$$\frac{{[ C ]}_{t}}{{[ C ]}_{0}} = \frac{n - 1}{n \cdot exp [ {kt ( n - 1 ) [ C ]}_{0} ] - 1} .$$

The next step was to relate the equation above to the experimental data we collected. For each kinetic curve, at time 0, after time t, and at the end of the reaction (t goes to ∞), the normalized fluorescence intensities were I₀, Iₜ, and I_{∞}, respectively. Hence by proportionality—

$$\frac{{[ C ]}_{t}}{{[ C ]}_{0}} = \frac{{I}_{∞} - {I}_{t}}{{I}_{∞} - {I}_{0}} . \qquad ( 12 )$$

Thus, equating equations (11) and (12), we obtained—

$$\frac{{I}_{∞} - {I}_{t}}{{I}_{∞} - {I}_{0}} = \frac{n - 1}{n \cdot exp [ {kt ( n - 1 ) [ C ]}_{0} ] - 1} \qquad ( 13 )$$

$${I}_{t} = {I}_{∞} - ( {I}_{∞} - {I}_{0} ) \cdot \frac{n - 1}{n \cdot exp [ {kt ( n - 1 ) [ C ]}_{0} ] - 1} .$$

The equation (13) was used to fit the normalized kinetic curve with time to obtain the rate constant k.

### Example 1. Design of a Large and Gated DNA Channel (LGC)

Representative DNA nanostructures as a large and gated channel (LGC) were designed using nanoarchitecture principles. In reference DNA nanopores (Langecker, M. et al. Synthetic lipid membrane channels formed by designed DNA nanostructures. Science 338, 932-936, (2012); Göpfrich, K. et al. Large-conductance transmembrane porin made from DNA origami. ACS Nano 10, 8207-8214, (2016); Krishnan, S. et al. Molecular transport through large-diameter DNA nanopores. Nat. Commun. 7, 12787, (2016); Diederichs, T. et al. Synthetic protein-conductive membrane nanopores built with DNA. Nat. Commun. 10, 5018, 1-11, (2019); Thomsen, R. P. et al. A large size-selective DNA nanopore with sensing applications. Nat. Commun. 10, 5655, (2019)), duplex helices are aligned parallel in lattice-fashion, as dictated by the caDNAno design software (Douglas, S. M. et al. Rapid prototyping of 3d DNA-origami shapes with cadnano. Nucleic Acids Res. 37, 5001-5006, (2009)). The duplexes are routed ‘vertically’ which is at 90 degree relative to the membrane plane (FIG. 1a). This arrangement places the majority of DNA into an extra-membrane cap region with multi-duplex layer-thick channel walls while restricting the amount of DNA available for forming a wide membrane-embedded pore (FIG. 1a). The small lateral footprint of the pore also limits the number of attachment points for lipid anchors required for efficient pore insertion into bilayer membranes. To overcome these restrictions, we opted to route the component helices ‘horizontally’ to the membrane by using the free-form software Tiamat (FIG. 1b-c) (Williams, S. et al. in DNA computing Lecture notes in computer science Ch. Chapter 8, 90-101 (2009)).

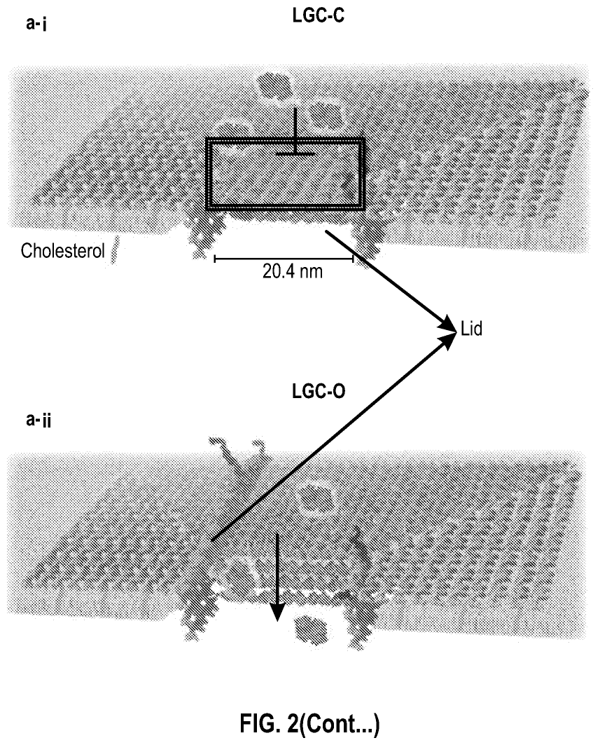

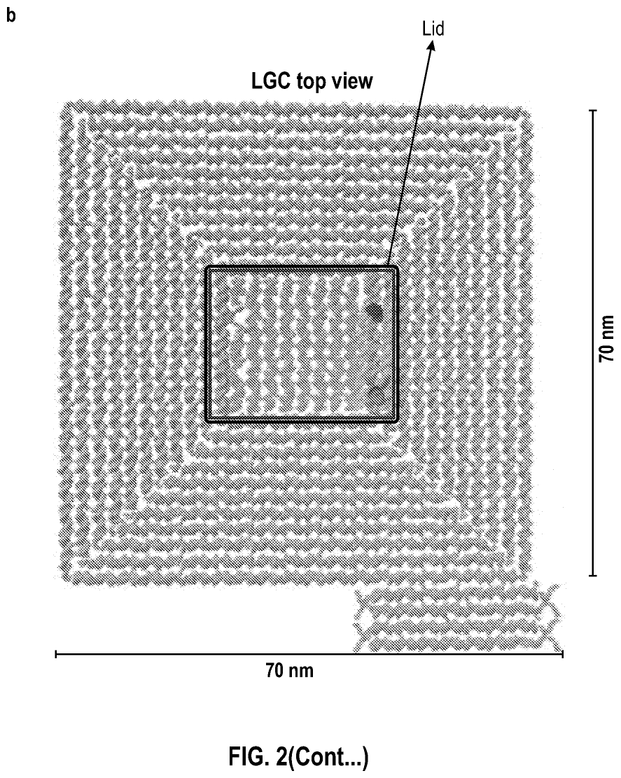

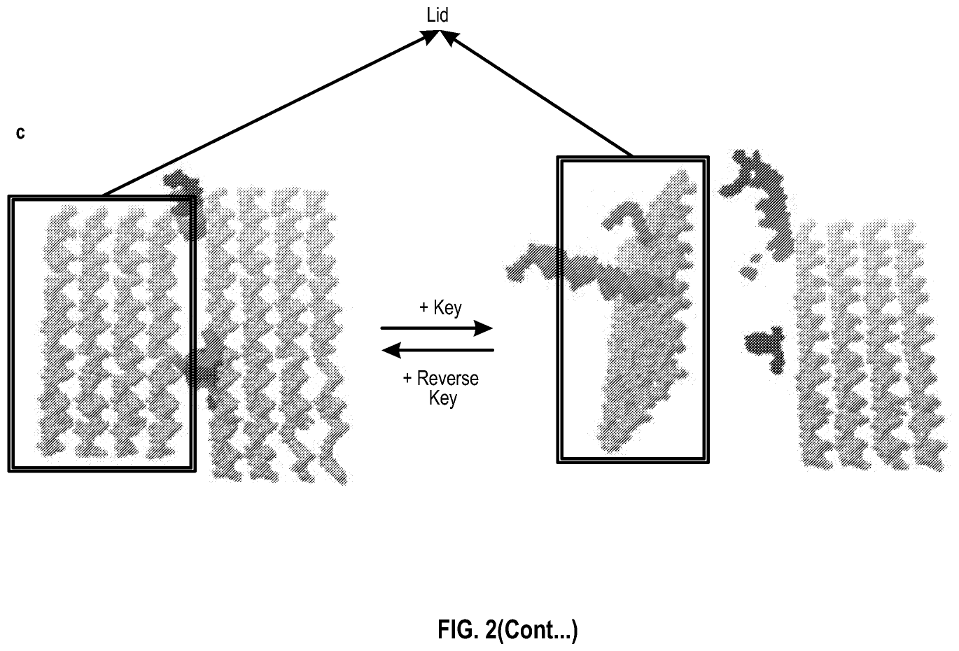

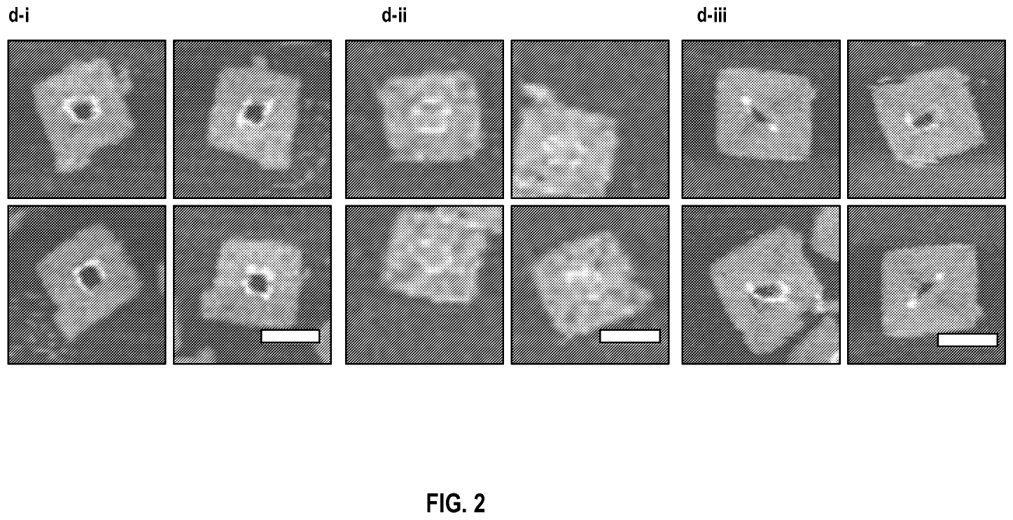

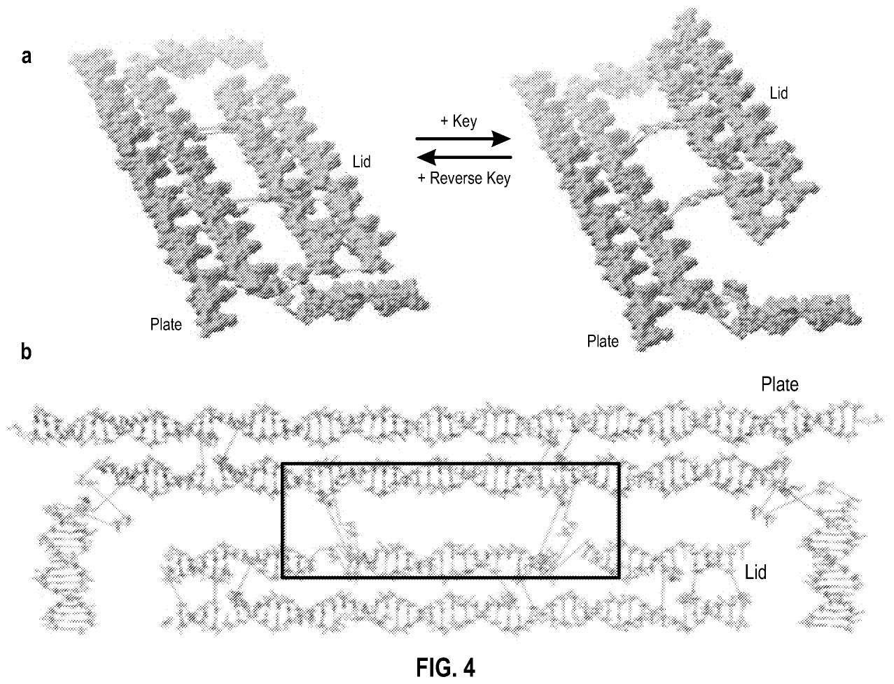

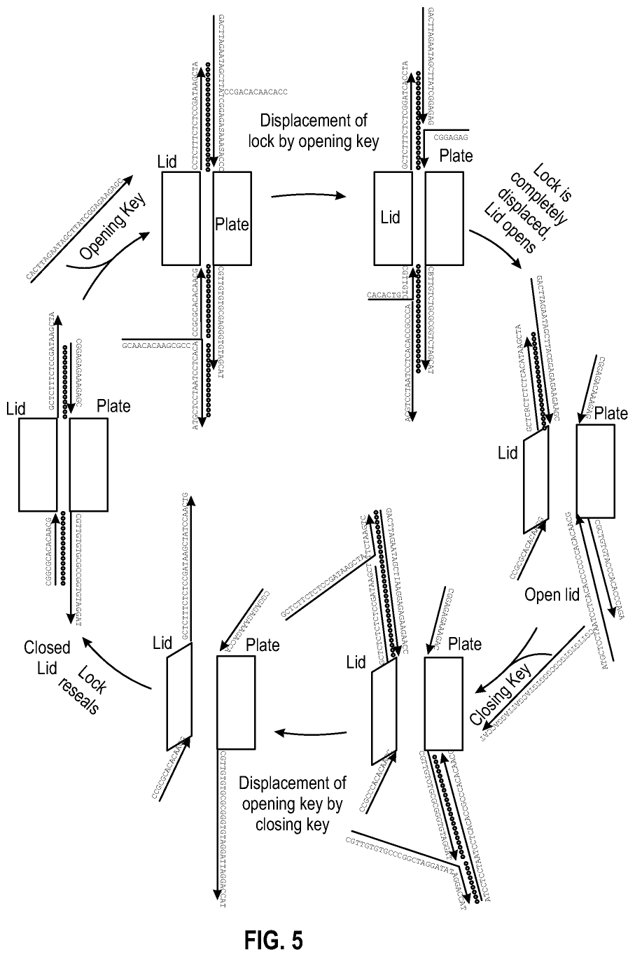

Using the horizontal routing, we rationally designed a channel featuring a 20.4 nm×20.4 nm-wide channel lumen into a single-duplex layer DNA origami plate of 70 nm×70 nm external dimensions (FIG. 2a, b). A four-duplex deep channel spanned the bilayer membrane while the large base plate sat on top of the bilayer (FIG. 2a). By drastically expanding the footprint of the extra-membrane cap, 64 hydrophobic cholesterol anchors were accommodated (FIG. 2a, FIG. 3) around the channel lumen on the bottom surface of the base plate, for efficient membrane insertion. As a further advantage of the design, the 416 nm²-wide channel can be reversibly closed and opened with a square lid composed of horizontally routed DNA duplexes to yield the closed channel LGC-C (FIG. 2a-i) and the open version LGC-O (FIG. 2a-ii). To achieve this dynamic change, the lid was attached at one side to the channel base plate by flexible hinges (FIG. 4). The other lid side carried two single-stranded half locks which can hybridize with the complementary half locks at the base plate to form complete duplex locks (FIG. 2a-i, ii). To open the lock and lid of the closed channel (LGC-C), a pair of single-stranded DNA keys dissociated the locks to form the open channel (LGC-O) (FIG. 2c, FIG. 5). The opened lid can be switched back to close by a single stranded reverse key pair (FIG. 2c). This externally controlled mechanism was expected to reversibly switch the lid-gated channel between an open and closed state to regulate flow of large molecular cargo.

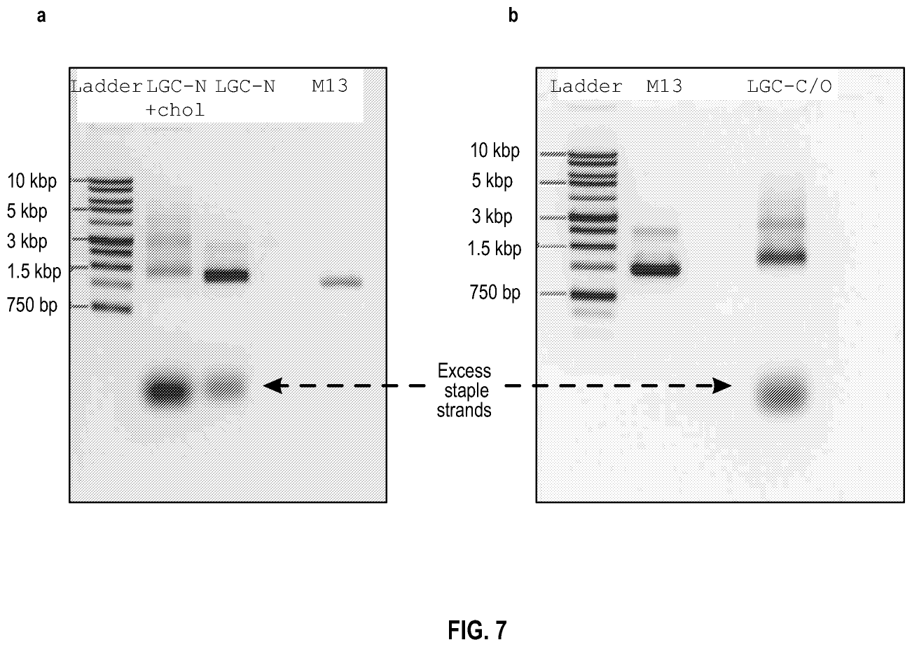

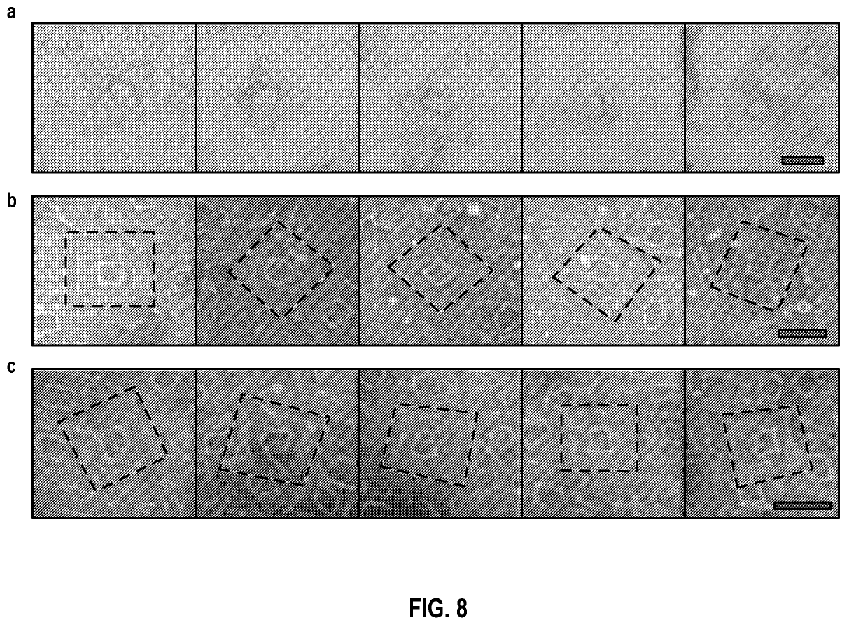

We first assembled non-lid version LGC-N and the nanostructures LGC-C and LGC-O carrying the lid in two states, without cholesterol anchors, by annealing the scaffold DNA with staple oligonucleotides (Table 1 and 2). OxDNA simulations (Šulc, P. et al. Design, optimization and analysis of large DNA and ma nanostructures through interactive visualization, editing and molecular simulation. Nucleic Acids Res. 48, e72-e72, (2020)) showed desirable formation of LGC-N, LGC-C, and LGC-O in solution (FIG. 6) and gel electrophoresis confirmed that single assembly products had formed (FIG. 7). Atomic force microscopy (AFM) (FIG. 2d, FIG. 6) and transmission electron microscopy (TEM) (FIG. 8) established the expected dimensions. For example, the AFM-derived external average side lengths of LGC-N was 78.0±2.9 nm for the square plate and 22.7±5.1 nm (n=65) (FIG. 2d-i, FIG. 6) for the square opening, close to the expected values of 70 nm and 20.4 nm, respectively. The elevations at the nanostructure center stemmed from the four duplex-high channel walls extending from LGC bound top-down to the mica substrate. By comparison, the closed-lid LGC-C featured no central opening, and the side-lengths were 77.4±2.6 nm for the plate and 22.7±1.9 nm for the channel wall (n=11) (FIG. 2d-ii, FIG. 6). Similarly, open-lid LGC-O appeared partly closed as the lid can obstruct the channel opening and the dimensions were 78.4±3.5 nm and 21.8±2.5 nm (n=13) (FIG. 2d-iii, FIG. 6). The slightly larger-than-nominal dimensions of all LGC variants were likely due to the flattening of the negatively charged DNA plate on the positively charged mica surface, compression by the AFM tip, or both factors. The channels were lipid-tagged by incubating with cholesterol-modified DNA oligonucleotides that bound to designed sites at the underside of the large membrane cap (FIG. 2a-i, FIG. 3).

### Example 2. Nanopore Interaction and Insertion into Bilayer

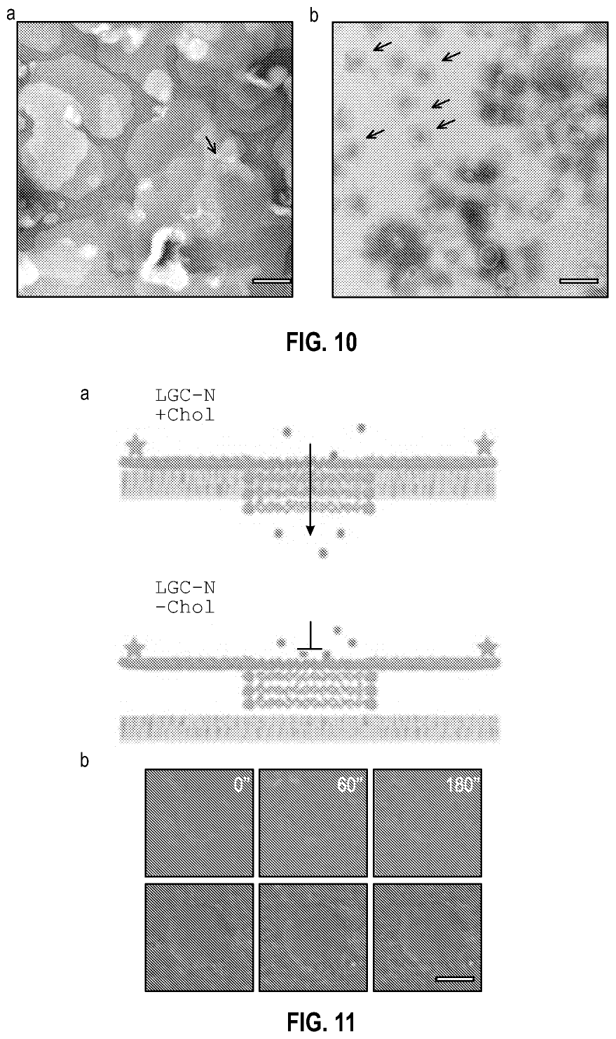

Following successful formation, it was tested whether the cholesterol-modified LGC can bind to and insert into lipid bilayers. To probe for membrane binding, channel variant LGC-N was added to small unilamellar vesicles (SUVs), and analyzed by gel electrophoresis in Mg²⁺ free buffer to avoid non-specific adsorption to chelating lipid head-groups. Increasing SUV concentrations led to concomitant gel electrophoretic upshift (FIG. 9a), suggesting that LGC-Ns can bind efficiently to lipid membranes of slowly migrating vesicles (Lanphere, C. et al. Design, assembly, and characterization of membrane-spanning DNA nanopores. Nat. Proto. 16, 86-130, (2020)). No gel-upshift was observed when LGC lacked cholesterol, underscoring its role for membrane binding. Cholesterol-mediated binding was also confirmed with direct visualization by TEM imaging (FIG. 9b, arrows; FIG. 10). The extent of membrane binding was probed by incubating Cy3-labeled LGC-N with giant unilamellar vesicles (GUVs) and examination with fluorescence microscopy. Co-localization of the cholesterol labeled LGC-Ns with the vesicle perimeter indicated successful membrane binding (FIG. 9c, FIG. 11).

To explore whether LGC-N punctured lipid bilayers, we tested the influx of membrane-impermeable Atto633 dye into the interior of GUVs (Göpfrich, K. et al. Large-conductance transmembrane porin made from DNA origami. ACS Nano 10, 8207-8214, (2016); Krishnan, S. et al. Molecular transport through large-diameter DNA nanopores. Nat. Commun. 7, 12787, (2016)). GUVs were placed in a solution of Atto633 and fluorescence microscopy tracked any changes in the fluorescence content of GUVs after adding LGC-N. The channel successfully inserted into and punctured membranes as indicated by Atto633 signals that increased within GUVs over the full incubation time (FIG. 9c-ii, top). A total of 59% of GUVs showed dye influx in case of LGC-N with cholesterol (LGC-N+Chol) (n=156) (FIG. 9d), which compared to solely 3.5% of GUVs incubated with LGC-N lacking cholesterol (LGC-N-Chol) (n=124) (FIG. 9c-ii, bottom), suggesting membrane insertion via cholesterol was key for puncturing the bilayer for cargo transport. The LGC mediated influx within 1 h after channel addition was considerably faster than 5-8 h required for previous DNA nanopores (Thomsen, R. P. et al. A large size-selective DNA nanopore with sensing applications. Nat. Commun. 10, 5655, (2019)). The faster flux was likely due to the larger pore lumen of LGC, the higher number of our inserted channels, or a combination of both.

The membrane spanning nature of the LGC-N was confirmed by electrical recordings (FIG. 9e-g). Individual channels were inserted into a planar DPhPC lipid bilayer that separated two chambers filled with electrolyte (1M KCl, 10 mM HEPES pH 7.6). To induce ion flow across an inserted channel, a transmembrane potential was applied. An ensuing steady current of 461 pA (FIG. 9e, +80 mV) indicated membrane insertion. LGC-N's average conductance value was high at 4.95±2.35 nS (n=15, SEM) (FIG. 9f) but in agreement with the wide channel lumen. Similarly, the linear relationship between current magnitude and voltage was expected for a channel lumen with vertical symmetry (FIG. 9g).

### Example 3. Reversibly Ligand Gated Lid-Controlled Transport of Small Molecule Cargo

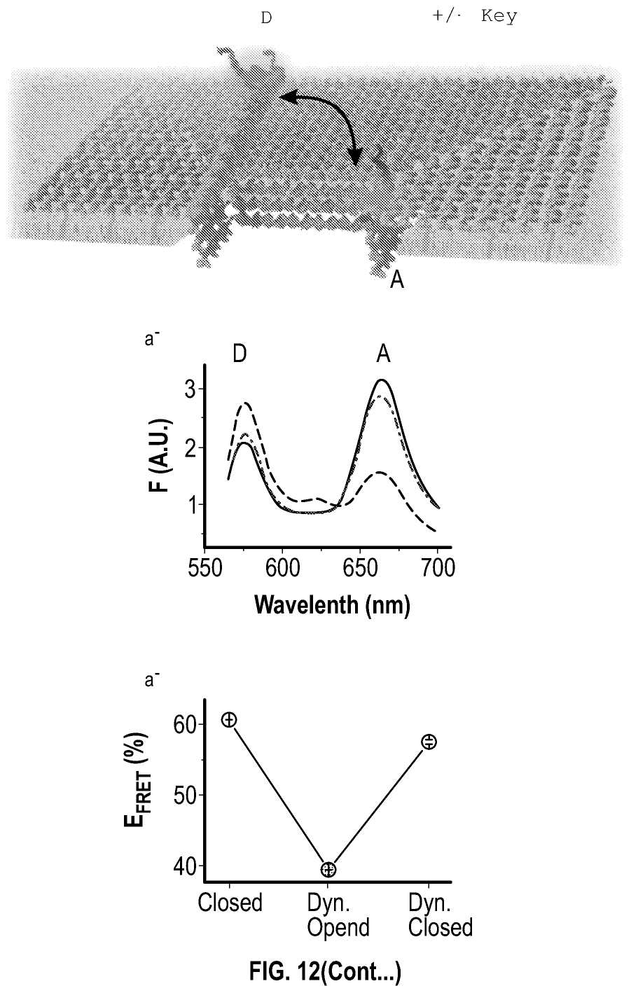

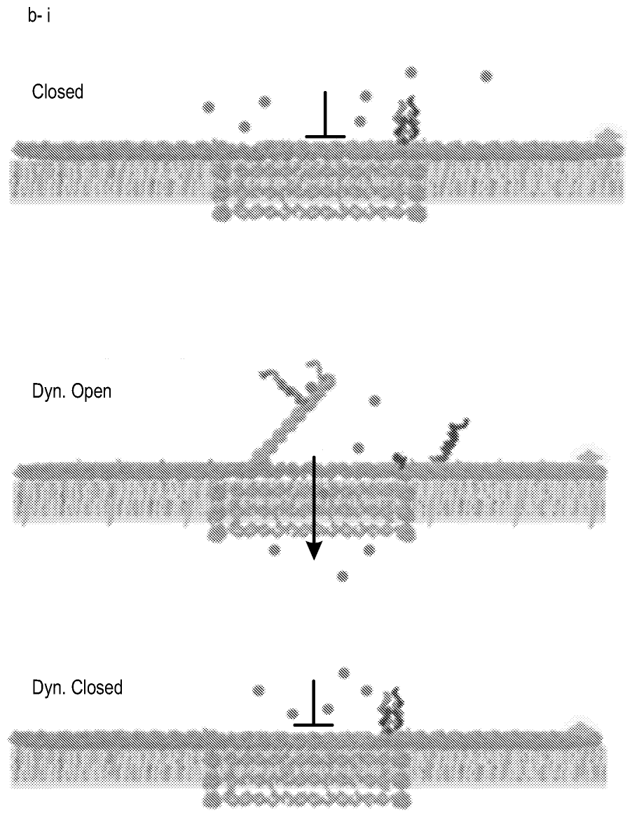

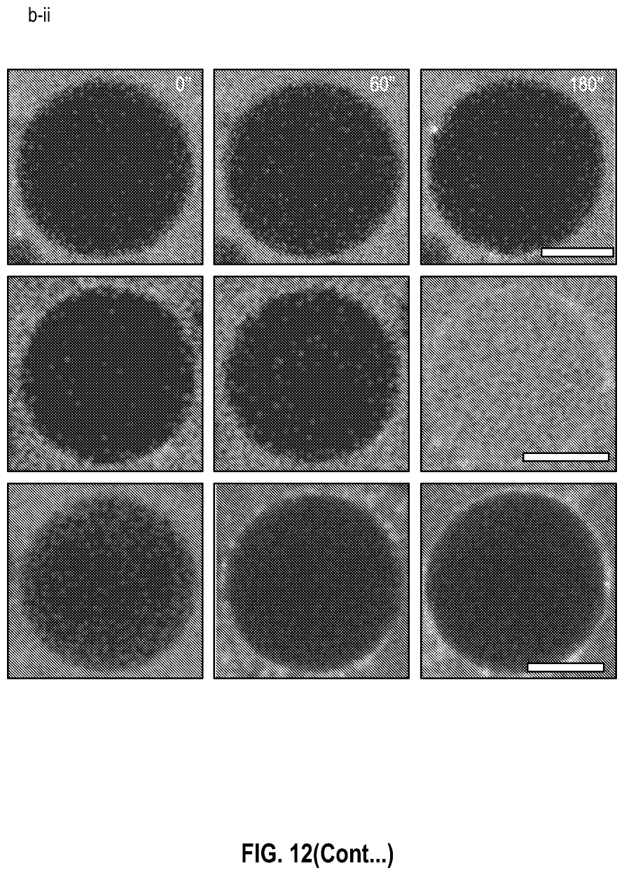

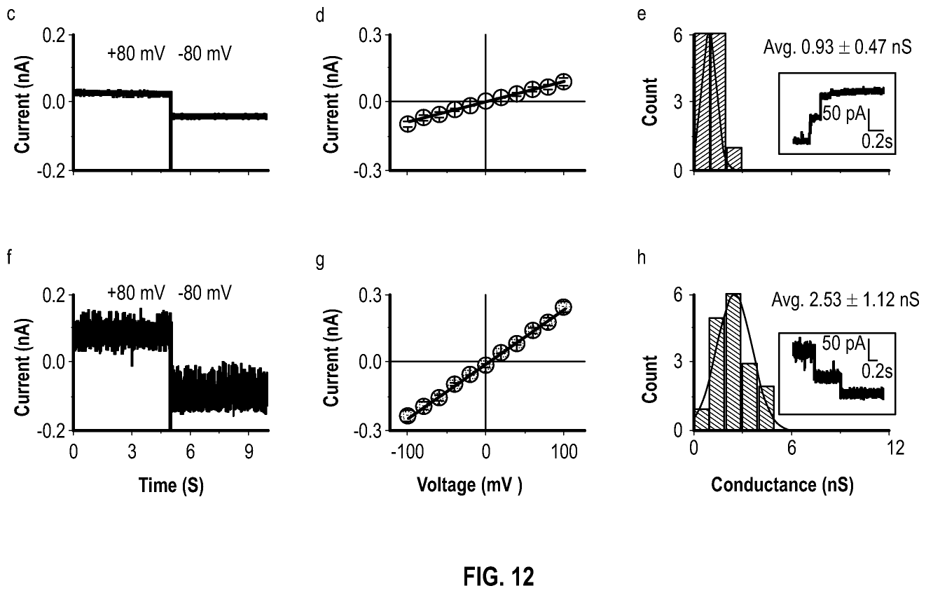

Unlike constitutively open pores, biological ion channels open solely upon specific stimuli, and then close by poorly understood ligand dissociation mechanism. The DNA nanostructures of this disclosure comprising a large gated channel were designed to achieve defined transport control via a toehold-mediated strand displacement reaction (Yurke, B., Turberfield, A. J., Mills, A. P., Simmel, F. C. & Neumann, J. L. A DNA-fuelled molecular machine made of DNA. Nature 406, 605-608, (2000); Guo, Y. et al. Recent advances in molecular machines based on toehold-mediated strand displacement reaction. Quant. Biol. 5, 25-41, (2017)) between lock and key (FIG. 2a-c, FIG. 5). We tested the reversible opening and closing of the lid using Fluorescence Resonance Energy Transfer (FRET). A Cy3 donor and Cy5 acceptor dye was placed in the lid and the base plate, respectively (FIGS. 12a-i, D and A). Due to spatial proximity between the dyes, FRET occurred in the closed state (Closed or LGC-C) as reflected by the low donor fluorescence at λₘₐₓ^{Cy3}=564 nm and high acceptor fluorescence at λₘₐₓ^{Cy5}=670 nm (FIG. 12a-ii, solid red line; FIG. 12a-iii). Opening the lid by adding key (Dyn. Open) increased the distance between the reporter dyes and resulted in a low FRET, a higher donor emission, and a drop in the acceptor emission (FIG. 12a-ii, blue line;

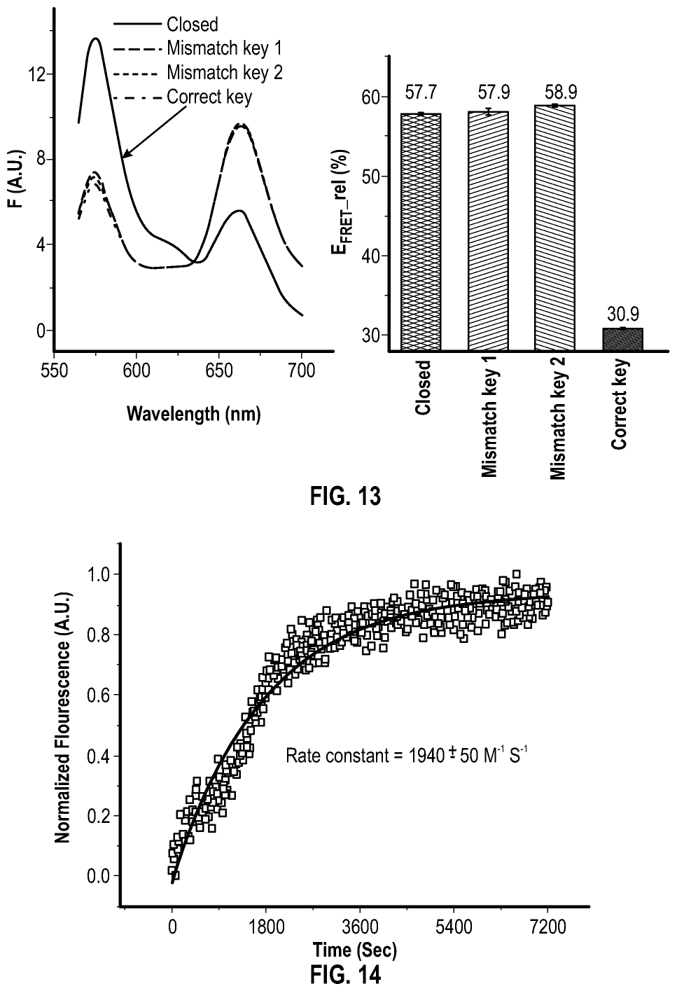

FIG. 12a-iii). Closing the lid back from its open state using reverse key (Dyn. Closed) restored FRET, lowered donor fluorescence, and increased acceptor fluorescence (FIG. 12a-ii, dashed red line; FIG. 12a-iii). The key-controlled switch was sequence-specific as confirmed by a mismatch opening key (FIG. 13). Analysis of the kinetic FRET signal revealed that lid opening followed second-order kinetics (Liu, M. et al. Rapid photoactuation of a DNA nanostructure using an internal photocaged trigger strand. Angew. Chem. Int. Ed. 57, 9341-9345, (2018)) at a rate constant of 1940±50 M⁻¹ s⁻¹ until transport completion after 2 h (FIG. 14).

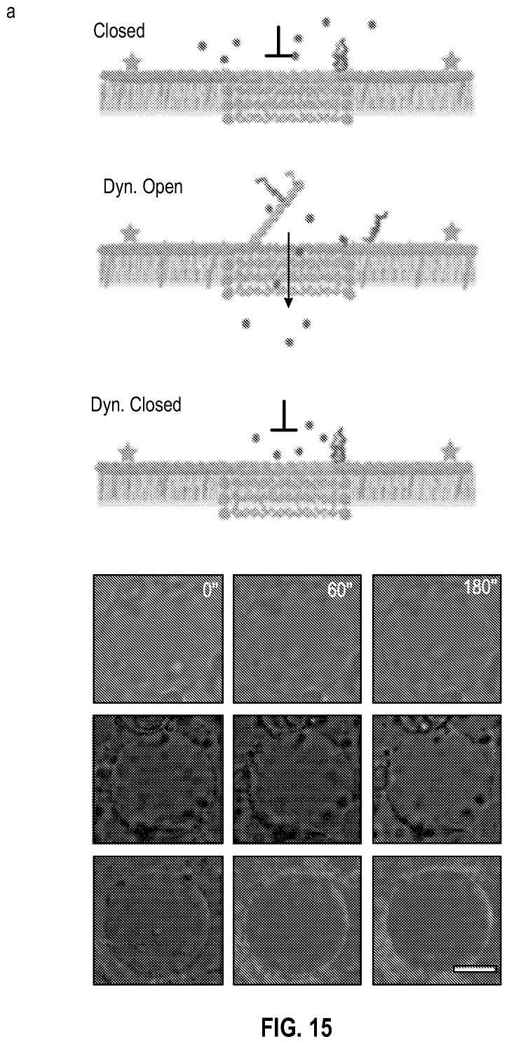

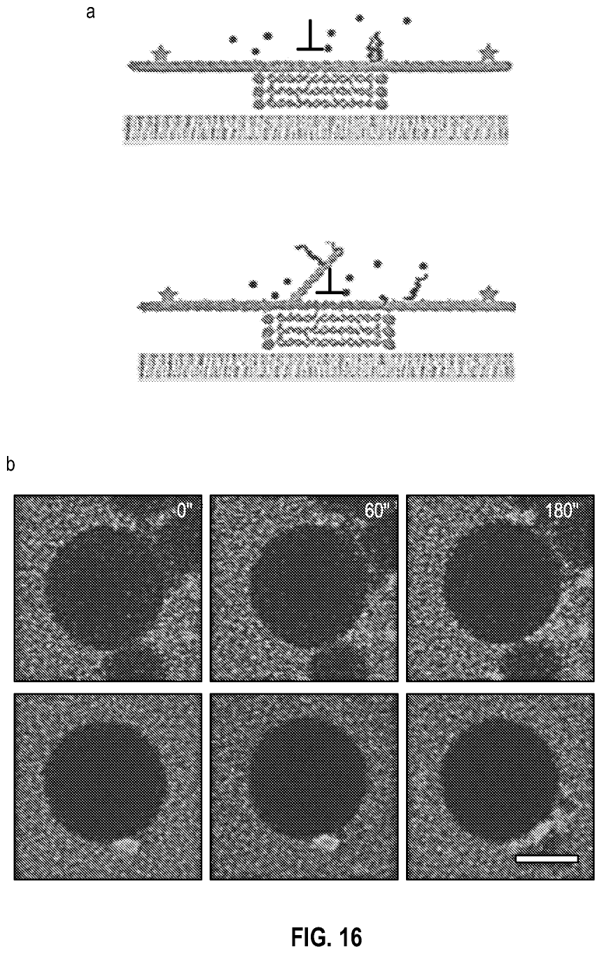

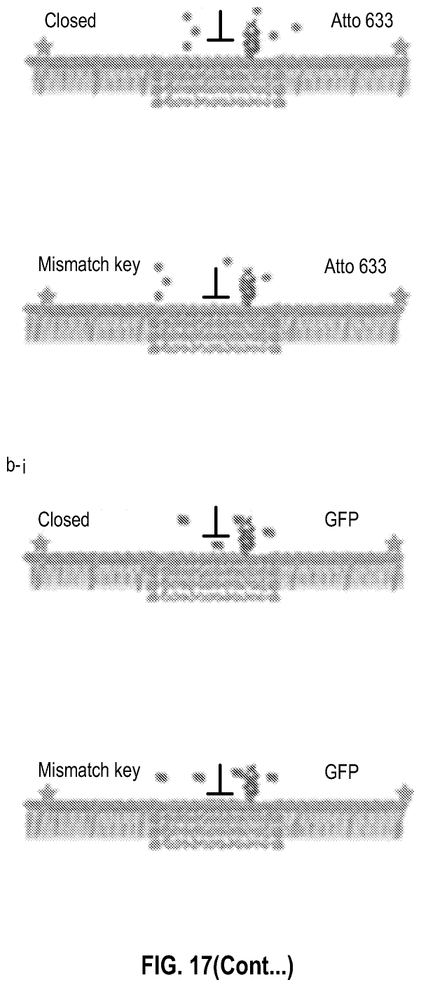

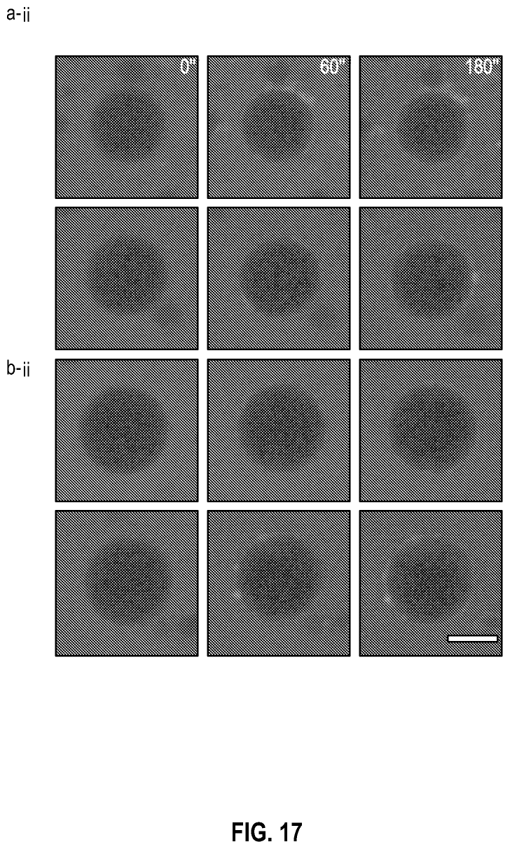

Next, we utilized the GUV-dye influx assay to demonstrate controlled transport through the channel. GUVs were immersed in a solution of Atto633, and influx was probed via fluorescence microscopy. In control analysis, cholesterol modified LGC-C bound to the vesicle membranes whereas the non-cholesterol version did not show binding, as indicated by presence or absence of fluorescent rings around the GUV perimeter (FIG. 15, 16). Probing of the fluorescence intensity within GUVs established that membrane-bound LGC-C did not lead to dye flux into the vesicles (FIG. 12b, top). This implied that the lid completely blocked transport through the LGC channel. Upon addition of keys, however, dye fluxed inside GUVs demonstrating that the opened-lid channel was transport active (FIG. 12b, middle). To confirm that the channel can be shut back to cease transport function, we dynamically closed the lid by incubating with reverse key. Indeed, dye influx stopped close to the level of LGC-C (FIG. 12b, bottom). Negative control experiments with the mismatch keys did not show similar Atto633 dye influx (FIG. 17). These data clearly confirm that the lid can be dynamically opened and closed specially with external triggers, something which has not be achieved before by any means.

Single-channel current recordings probed the characteristics of cholesterol modified closed lid (LGC-C) and open-lid (LGO-O) LGCs (FIG. 12c-h), and the dynamic transitions between the two. As would be expected from the steric blockade by the closed lid, LGC-C featured a far smaller current and corresponding conductance at 0.93±0.47 nS (n=13, SEM) (FIG. 12c, e) than had been obtained with no-lid LGC at 4.95±2.35 nS (n=15, SEM) (FIG. 9f). The presence of the lid did not affect linear voltage-current dependence (FIG. 12d). The small residual current of LGC-C might stem from ion leakage either through the periphery of the lid or through the DNA duplexes of the lid, or both. By comparison, the conductance of LGC-O at 2.53±1.12 nS (n=17, SEM) (FIG. 12f, h) was twice the value of LGC-C (FIG. 12e), reflecting that the open lid allowed more ion transport. However, it was still half the value of non-lid LGC in agreement with residual blockade by an open lid (FIG. 9f).

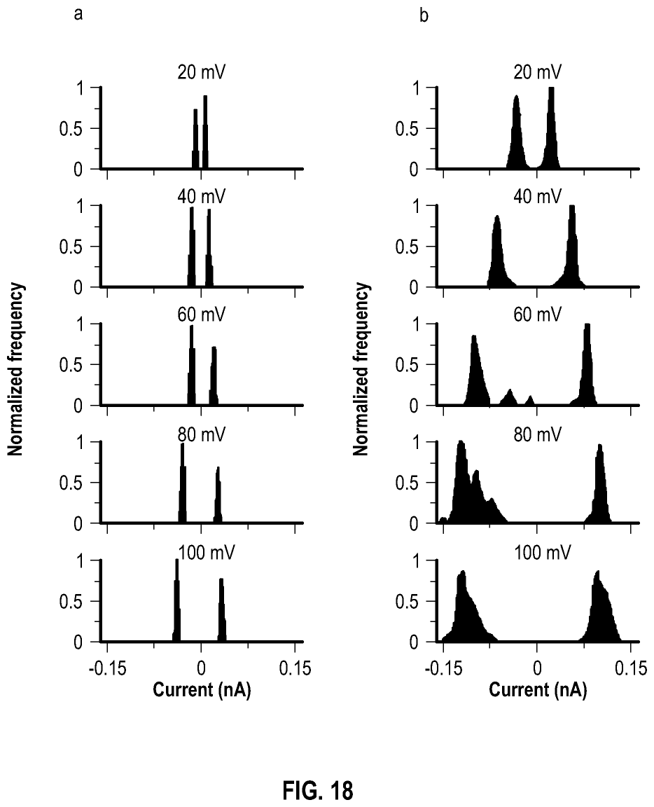

Single-channel analysis provided further insight into the dynamic nature of the LGC lid. Current traces of the single LGC-O channels had more high-frequency fluctuations (FIG. 12f, FIG. 18b) than the less noisy trace of LGC-C (FIG. 12c, FIG. 18a). The current fluctuations likely reflected the dynamic movements of the lid to and from the channel base in LGC-O compared to the static lid in LGC-C. Indeed, electrophoretically driving the lid of LGC-O to its base plate via a negative potential led to an additional current level at −19.0 pA next to the main conductance peak at −95.2 pA (FIG. 18). By contrast, moving the lid away via positive potentials only had a single main peak at 90.8 pA (FIG. 18). The voltage-dependent noise strikingly revealed nanomechanical-dynamic changes of a DNA structure at the single-molecule level.

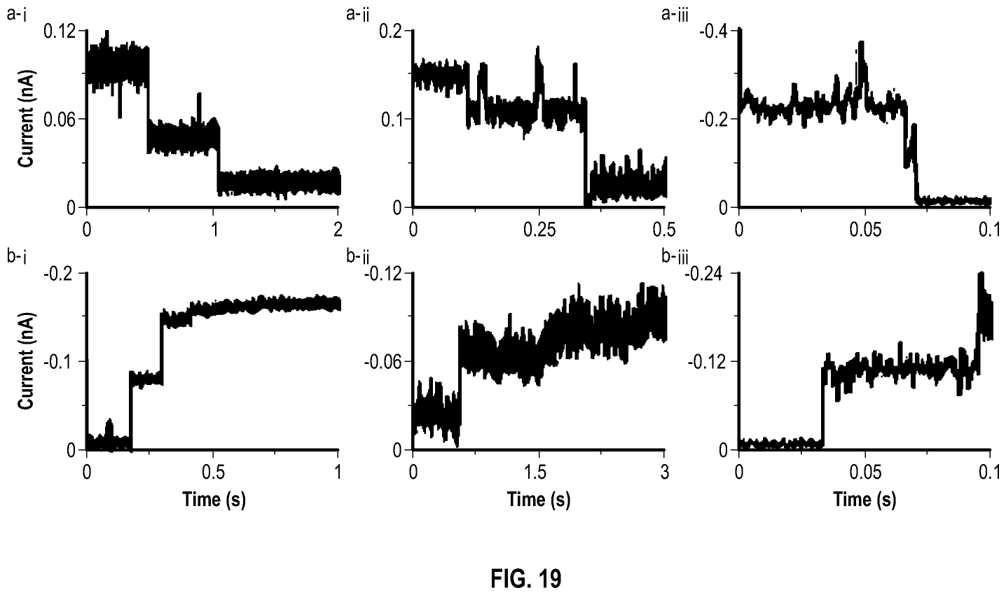

Single-molecule analysis revealed insight into the closing and opening mechanism. LGC-O could be closed by adding reverse key, as demonstrated by the transition from an open state current at 97.4 pA to the lower-amplitude closed state at 17.7 pA (FIG. 12h, inset; FIG. 19). This transition occurred in a stepwise fashion, likely because the two lid locks were closed one after the other leading to an intermediate state of one closed and one open lock (FIG. 2c-i). Similarly, LGC was opened by adding key as indicated by a switch from the closed-state at an amplitude of 8.8 pA to the open-state at amplitude 147.4 pA (FIG. 12e, inset; FIG. 19). This transition occurred also in a stepwise fashion. Our data revealed the opening and closing mechanism of the LGC channel in unprecedented detail and indicated ways to fine-tuning opening by varying the lock number.

### Example 4. Transport of Folded Proteins Across the LGC

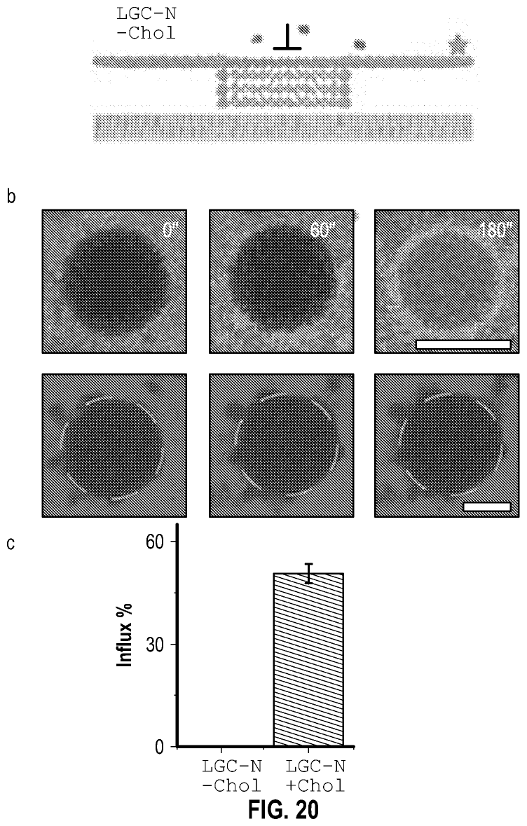

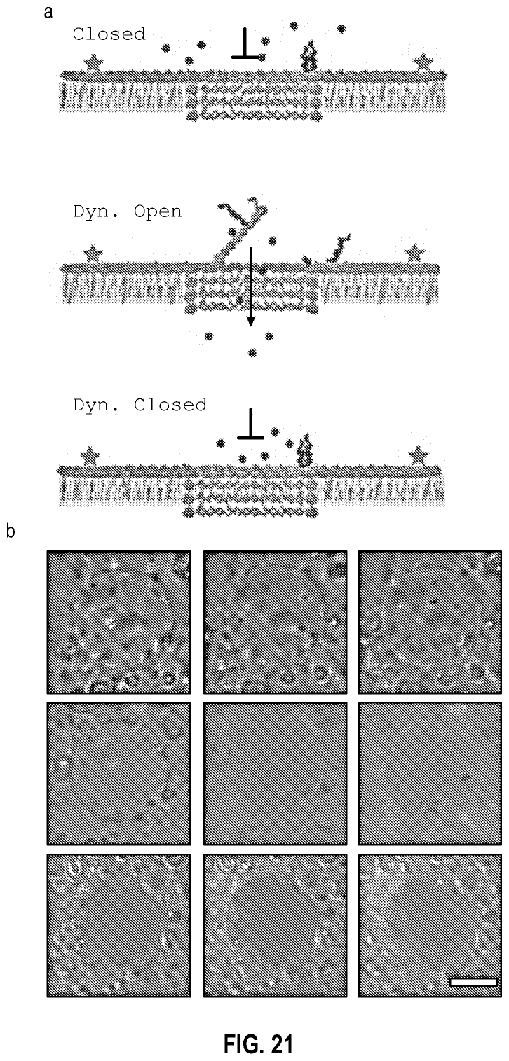

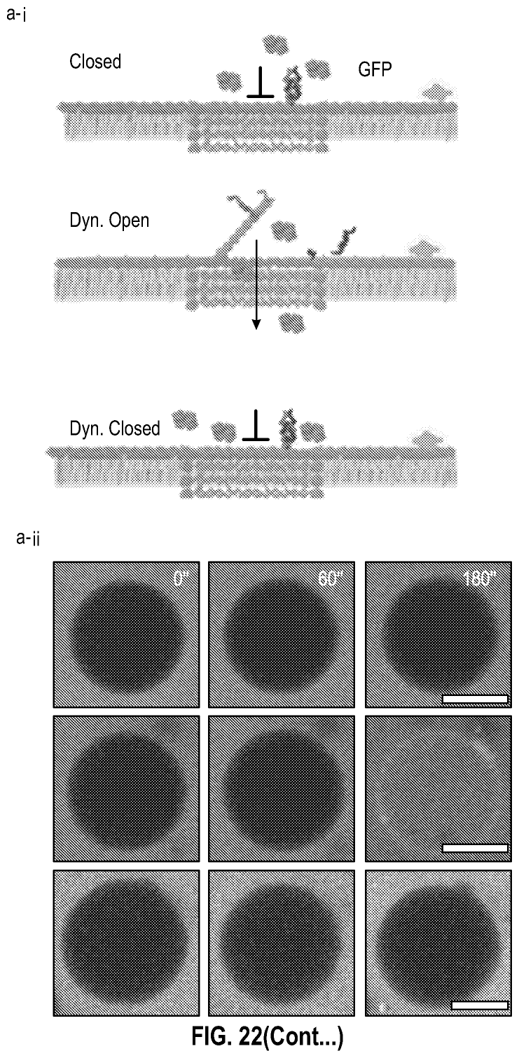

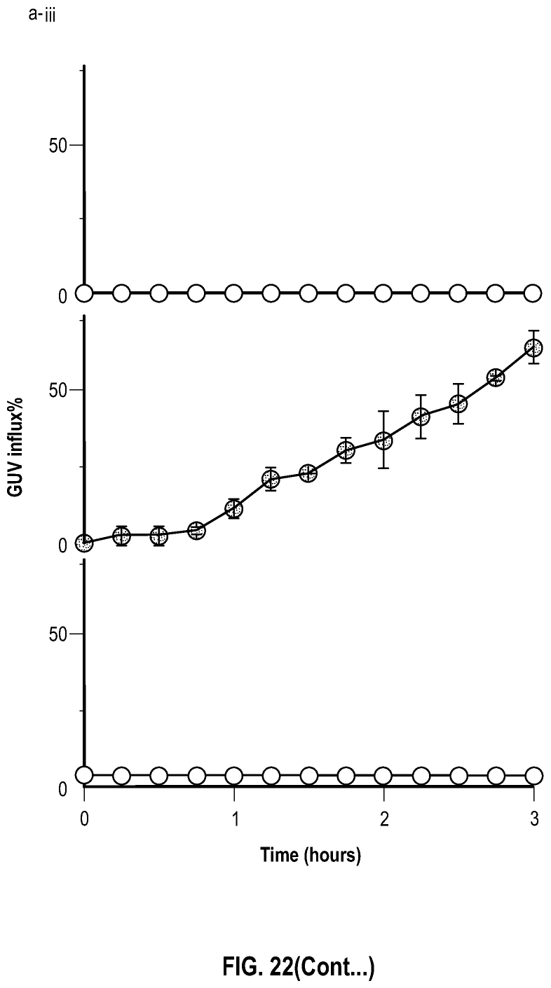

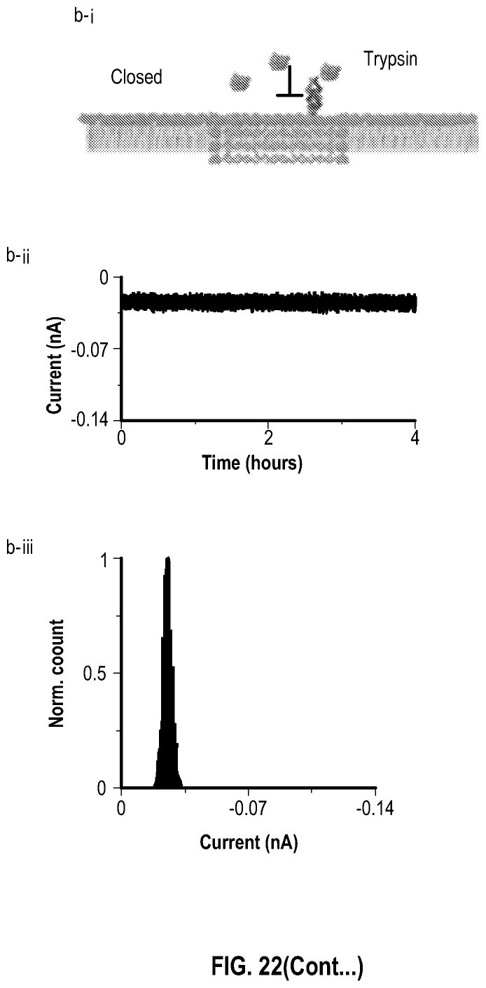

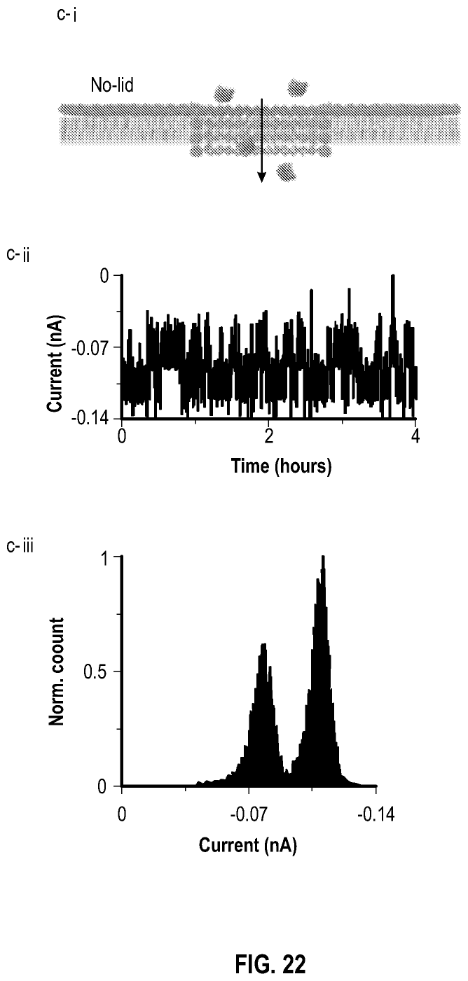

We finally exemplified the power of LGC by regulating the flux of folded proteins across membranes via defined lid opening and closing. GUVs were immersed in a solution of green fluorescent protein (GFP, hydrodynamic diameter=5.6 nm (Terry, B. R., Matthews, E. K. & Haseloff, J. Molecular characterization of recombinant green fluorescent protein by fluorescence correlation microscopy. Biochem. Biophys. Res. Commun. 217, 21-27, (1995)). Cholesterol modified channels bound to GUV membranes whereas unmodified versions did not bind (FIG. 20, 21). When influx was monitored by determining fluorescence intensity in GUVs, control channel LGC-N without lid led to transport across membranes (FIG. 20). By contrast, LGC-C blocked protein flux (FIG. 22a, top). However, adding key opened up the transport function (FIG. 22a, middle), whilst reverse key addition again stopped protein influx (FIG. 22a, bottom). Although atto-633 and GFP differs in hydrodynamic diameter, both show similar rate of influx %, perhaps because the LGC pore being large enough compared to both Atto 633 and GFP. Moreover, the no lid and dynamically opened lid LGC pores showed similar rate of influx % whereas the closed lid pore showing no influx % indicated that the open lid was equivalent to no lid in terms of passage of molecules through the LGC channel.

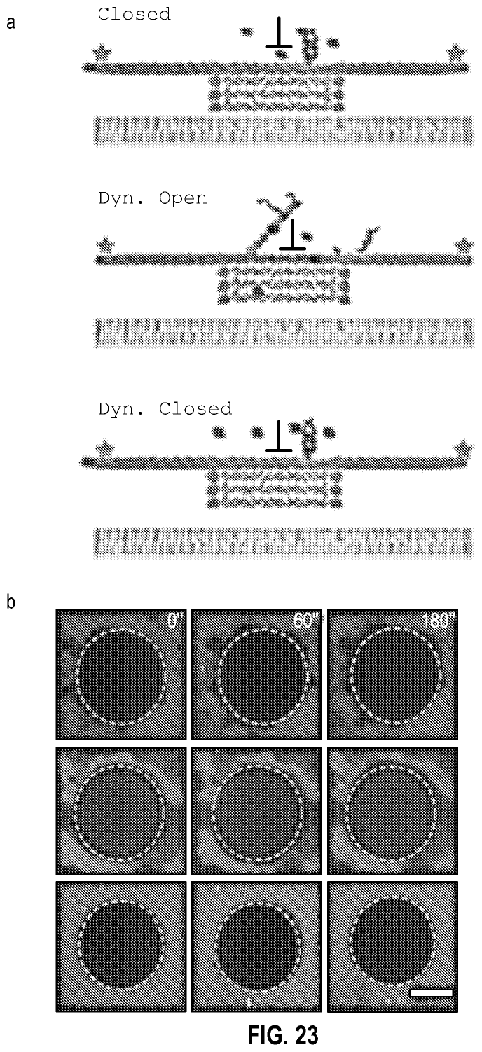

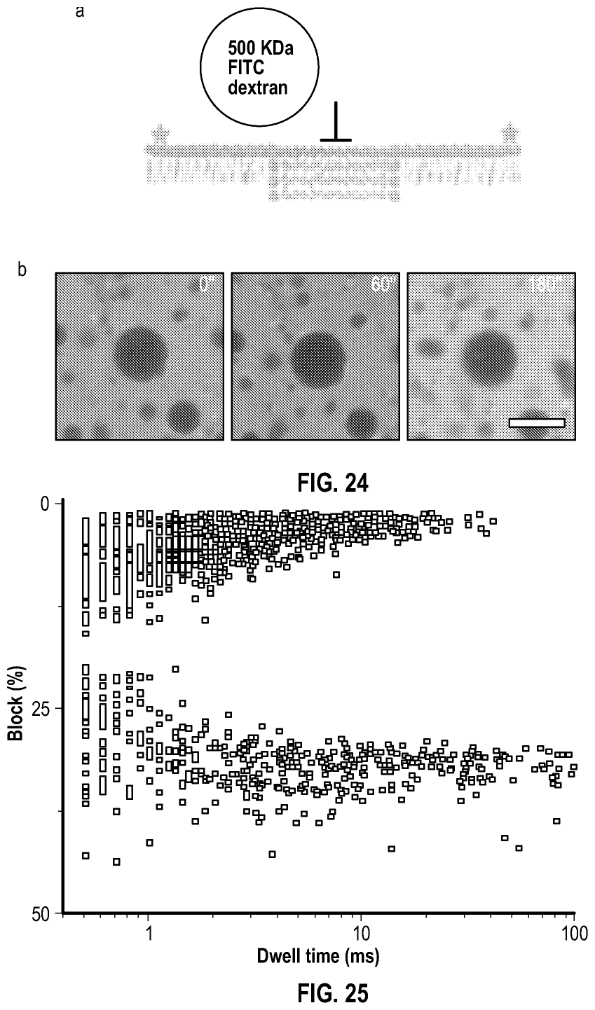

Negative control experiments with 500 kDa FITC-dextran featuring a hydrodynamic diameter of 31.8 nm (Armstrong, J. K., Wenby, R. B., Meiselman, H. J. & Fisher, T. C. The hydrodynamic radii of macromolecules and their effect on red blood cell aggregation. Biophys. J. 87, 4259-4270, (2004)) established that channel transport did not occur due to size-exclusions (FIG. 23); FITC-dextran also ruled out membrane rupturing. The successful data on the precisely timed transport of folded proteins and small organic dyes (FIG. 12b, FIG. 24) is something beyond the scope of biological and any previously engineered membrane channel.

The transport of folded protein across the membrane-nanopore lumen was further examined with single-channel current recordings. As a model protein, we used trypsin (hydrodynamic diameter ˜4 nm) with net positive charge (pI 10.1, pH 7.6). No translocation events occurred upon addition of trypsin to LGC-C (FIG. 22b-i) as shown by relatively steady current flow (FIG. 22b-ii, iii). By contrast, with LGC-N channels trypsin led to translocation (FIG. 22c-i), as indicated by blockade events (FIG. 22c-ii, iii). When analyzed by their relative blocking amplitude, A, (FIG. 25), events clustered at 3.5±2.6% (Type I) and at 33.3±10.7% A (Type II). It was suggested that Type I events occurred due to brief interaction of the protein with the nanopore at the lumen opening, while in Type II events proteins fully translocated through the nanopore.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

All DNA and RNA sequences presented herein are oriented 5′->3′, unless noted otherwise.

Although the foregoing specification and examples fully disclose and enable certain embodiments, they are not intended to limit the scope, which is defined by the claims appended hereto.

All publications, patents and patent applications are incorporated herein by reference. While in the foregoing specification certain embodiments have been described, and many details have been set forth for purposes of illustration, it will be apparent to those skilled in the art that additional embodiments and certain details described herein may be varied considerably without departing from basic principles.

The use of the terms “a” and “an” and “the” and similar referents are to be construed to cover both the singular and the plural, unless otherwise indicated herein or clearly contradicted by context.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the technology and does not pose a limitation on the scope of the technology unless otherwise claimed. No language in the specification should be construed as indicating any non-claimed element as essential to the practice of the technology.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the embodiment.

Embodiments are described herein, including the best mode known to the inventors. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this technology includes all modifications and equivalents of the subject matter recited in the claims appended hereto as permitted by applicable law. Moreover, any combination of the above-described elements in all possible variations thereof is encompassed by embodiments unless otherwise indicated herein or otherwise clearly contradicted by context.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

We claim:

1. A nanostructure comprising:
a) a base plate sheet comprising a single-duplex layer comprised of DNA origami that is configured in a planar arrangement,
b) a pore configured within the center of the base plate sheet, and
c) a multi-duplex layer-thick wall comprising a single-duplex layer comprised of DNA origami that extends downward from the base plate sheet on the periphery of the pore, wherein the base plate sheet comprises a single-stranded DNA region on the bottom surface.
d) a lipid membrane anchor covalently linked to a single-stranded DNA sequence which is partially complementary to the single-stranded DNA region of the base plate sheet on the bottom surface.

2. (canceled)

3. The nanostructure of claim 3, wherein the lipid membrane anchor is selected from cholesterol, or a fatty alcohol.

4. The nanostructure of claim 3, wherein the fatty alcohol is selected from tetradecanol, hexadecanol, or octadecanol.

5. The nanostructure of claim 1, wherein the multi-duplex layer-thick wall is a four-duplex layer-thick wall.

6. The nanostructure of claim 1, wherein the multi-duplex layer-thick wall is about perpendicular to the base plate sheet.

7. The nanostructure of claim 1, further comprising a lid configured to block the pore in a closed position, comprising a single-duplex layer comprised of DNA origami, and one or a plurality of flexible hinge sequences connecting the lid to the base plate sheet,
wherein each of the plurality of flexible hinge sequence independently comprises a single-stranded region,
wherein the lid further comprises one or a plurality of a first half-lock, wherein each first half-lock independently comprises a single-stranded DNA sequence, and
wherein the base plate sheet comprises one or a plurality of a second half-lock comprising a single-stranded DNA sequence, wherein the first half-lock and the second half-lock are partially complementary to each other.

8. (canceled)

9. The nanostructure of claim 7, wherein each flexible hinge sequence comprises a 4-nucleotide single stranded region.

10. (canceled)

11. (canceled)

12. The nanostructure of claim 7, further comprising a key single-stranded DNA sequence which is partially complementary to the regions of complementarity between the first half-lock and the second half-lock.

13. The nanostructure of claim 7, further comprising a reverse key single-stranded DNA sequence which is partially complementary to the regions of complementarity between the key single-stranded DNA sequence and the first or second half-lock.

14. The nanostructure of claim 7, wherein the lid is in a closed position such that the pore is blocked.

15. The nanostructure of claim 7, wherein the lid is in an open position such that the pore is not blocked.

16. (canceled)

17. The nanostructure of claim 7, wherein one of the first half-lock and the second half-lock comprises a donor fluorophore, and the other comprises an acceptor fluorophore.

18. The nanostructure of claim 1, wherein the base plate sheet is square shaped and has a size of about 70 nm×70 nm.

19. The nanostructure of claim 1, wherein the pore is square shaped and has a size of about 20 nm×20 nm.

20. A composition comprising the nanostructure of claim 1 and a lipid bilayer.

21. The composition of claim 20, wherein the lipid bilayer is part of a cell membrane.

22. A method of delivering an agent through a lipid bilayer, the method comprising:
a) contacting an agent and a nanostructure of claim 1 having a pore in a closed configuration with a lipid bilayer, wherein the lipid membrane anchor forms a complex with the lipid bilayer,
b) presenting a key single-stranded DNA sequence to the nanostructure, wherein the lid opens and the pore is in an open configuration, and
c) traversing the agent through the pore through the lipid bilayer.

23. The method of claim 22, wherein the lipid bilayer is part of a cell.

24. (canceled)

25. The method of claim 22, wherein the agent is selected from a small hydrophobic molecule having a molecular weight of less than 1000 Da, an ion, or a folded protein which comprises a hydrodynamic radius of less than the pore size.

26. The method of claim 25, wherein the small hydrophobic molecule is a fluorophore.

27. (canceled)

28. (canceled)

29. (canceled)

30. (canceled)

31. (canceled)
